(19) United States
(12) Patent Application Publication
(10) Pub. No.: US 2025/0388945 A1
(43) Pub. Date: Dec. 25, 2025

(54) THERMOSTABLE GLYCOSYLTRANSFERASE VARIANTS

(71) Applicant: DANMARKS TEKNISKE UNIVERSITET, Kongens Lyngby (DK)

(72) Inventors: Ditte Hededam WELNER, Kongens Lyngby (DK); Gonzalo Nahuel Bidart COSTOYA, Kongens Lyngby (DK); Leila Lo LEGGIO, Kongens Lyngby (DK)

(21) Appl. No.: 18/837,991

(22) PCT Filed: Feb. 21, 2023
(86) PCT No.: PCT/EP2023/054318

§ 371 Date: Aug. 13, 2024

(30) Foreign Application Priority Data
Feb. 22, 2022 (EP) 22158070.7

(51) Int. Cl. C12P 19/40; C09B 62/343; C12N 9/42; D06P 1/00

(52) U.S. Cl. CPC C12P 19/40; C09B 62/3435; C12N 9/2445; C12Y 302/01021; D06P 1/0004; D06P 1/0048

(57) ABSTRACT

The present invention concerns glycosyltransferase enzyme mutants having improved half-lives and thermal stability compared to the parent enzyme UDP-glycosyltransferase (PtUGT) from the indigo producing plant Polygonum tinctorium/Persicaria tinctoria; and further provides a composition, kit, and methods employing these mutants for glycosylation of desired compounds, such as indoxyl compounds.

### FIELD OF THE INVENTION

The present invention concerns enzyme mutants having improved temporal, thermal, and chemical stability, compared to the parent enzyme glycosyltransferase (PtUGT1) from the indigo producing plant Polygonum tinctorium/Persicaria tinctoria; and their use in methods for glycosylation of desired compounds, such as indoxyl compounds and thereby providing a greener alternative to current industrial processes for colored fabrics and other products.

### BACKGROUND OF THE INVENTION

Glycosyltransferases, such as UDP-glycosyltransferases (UGTs), can be used in biotech applications to attach a sugar molecule to a vast variety of industrial chemicals (e.g. fragrances, dyes, food additives), thereby enhancing their solubility and decreasing volatility and toxicity. This can also be used to enhance the bioavailability of pharmaceuticals. However, natural glycosyltransferases are not particularly stable, making them economically infeasible to use on industrial scale.

### SUMMARY OF THE INVENTION

In one aspect, the present invention provides a polypeptide having glycosyltransferase activity (enzyme classification EC: 2.4.1.-), wherein the amino acid sequence of said polypeptide has at least 75% sequence identity with SEQ ID NO. 2, and wherein said amino acid sequence comprises (i) one or more amino acid residue substitutions selected from: E75P, Q86K, S110V, 1188L, G222D, G296L, V297G, F381V, T388A, S413K and G430K with respect to SEQ ID NO 2, and/or (ii) amino acid residue substitutions T388C and A399C with respect to SEQ ID NO 2.

In one preferred embodiment, the present invention provides a polypeptide having glycosyltransferase activity (enzyme classification EC: 2.4.1.-), wherein the amino acid sequence of said polypeptide has at least 75% sequence identity to SEQ ID NO.2, and wherein said amino acid sequence comprises amino acid residue substitutions E75P, Q86K, S110V, I188L, G222D, G296L, V297G, S413K, G430K, and T388A with respect to SEQ ID NO 2.

In another preferred embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, or 97% sequence identity to SEQ ID NO. 2, and wherein said amino acid sequence comprises amino acid residue substitutions E75P, Q86K, S110V, I188L, G222D, G296L, V297G, S413K, G430K, F381V and T388A with respect to SEQ ID NO 2.

In another preferred embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, or 97% sequence identity to SEQ ID NO. 2, and wherein said amino acid sequence comprises amino acid residue substitutions E75P, Q86K, S110V, I188L, G222D, G296L, V297G, S413K, G430K, F381V, T388C, and A399C with respect to SEQ ID NO 2.

In a second aspect, the invention provides a composition comprising (i) a polypeptide of the present invention having glycosyltransferase enzyme, (ii) a compound comprising a reactive group, and (iii) a nucleotide sugar.

In a third aspect, the invention provides a kit of parts comprising (i) a polypeptide of the present invention having glycosyltransferase enzyme, and (ii) a polypeptide having beta-glucosidase enzyme activity (enzyme classification EC 3.2.1.21).

In a fourth aspect, the invention provides a method for glycosylating a compound, comprising the steps of
a. providing (i) a compound comprising a reactive group, (ii) a polypeptide of the present invention having glycosyltransferase enzyme, and (iii) a nucleotide sugar,
b. mixing the components provided in step (a)
c. letting the mixture react to obtain a glycosylated compound.

In a fifth aspect, the invention provides a method for dying a product, comprising the steps of
d. providing (i) an indoxyl compound, (ii) a polypeptide of the present invention having glycosyltransferase enzyme, (iii) a nucleotide sugar, and (iv) a polypeptide having beta-glucosidase enzyme activity (enzyme classification EC 3.2.1.21),
e. mixing components (i), (ii), and (iii) provided in step (a), preferably at reaction conditions wherein less than 2% free oxygen is present,
f. letting the mixture react to obtain a soluble glycosylated indoxyl dye-precursor,
g. mixing said dye precursor with said product and said beta-glucosidase under reaction conditions wherein free oxygen is present, to obtain a dyed textile.
wherein said product is elected from yarn, textiles, and fabrics,

In a sixth aspect, the invention provides use of a polypeptide of the present invention having glycosyltransferase enzyme, for glycosylating a compound, wherein said compound comprises a reactive group.

### Description of the Invention

### BRIEF DESCRIPTION OF THE FIGURES

FIG. 1: Scheme of glycosylation reactions catalyzed by UDP-glycosyltransferase.

FIG. 2: (A) Current industrial process involving chemically synthesized indigo and addition of reducing agents (e.g. sodium dithionite) to the indigo vat for reduction to dye-competent, soluble leucoindigo. In the proposed chemo-enzymatic process, indoxyl is glucosylated at the C3 hydroxyl group giving indican as product. The glucoside, indican, is stable and can be stored. The glucosyl group is removed only on fabric, during the dyeing step, allowing the regenerated indoxyl to oxidize to indigo on the fabric. No reducing agent is required when dyeing with indican. BGL=β-glucosidase; UGT=UDP-dependent glycosyltransferase. (B) Glucose acts as a protecting group for indoxyl. Removal of the glucose by a β-glucosidase releases indoxyl which can be further oxidized by air exposure.

FIG. 3: (A) and (B) Changes in the melting temperatures of mutants designed based on hypothetical disulfide bridge formation, in respect to PtUGT1 WT.

FIG. 4: Changes in the melting temperatures of double mutants and a triple mutant combining the different beneficial mutations based on hypothetical disulfide bridge formation, in respect to PtUGT1 WT.

FIG. 5: (A) and (B) Changes in the melting temperatures of mutants designed based on consensus mutagenesis, in respect to PtUGT1 WT.

FIG. 6: (A) and (B) Changes in the melting temperatures of mutants combining different beneficial mutations based on consensus mutagenesis, in respect to PtUGT1 WT.

FIG. 7: Relative activity of mutant 87, mutant 88 and mutant 90 at (A) 40° C., (B) 55° C., and (C) 60° C., in respect to PtUGT1 WT activity at 40° C.

FIG. 8: Relative activity of PtUGT1 WT, mutant 87, mutant 88 and mutant 90 after pre-incubation for different period of time at 45° C., in respect to the activity without pre-incubation at 45° C.

FIG. 9: Relative activity of PtUGT1 WT, mutant 87, mutant 88 and mutant 90 after pre-incubation for different period of time at room temperature (22° C.), in respect to the activity without pre-incubation at room temperature.

FIG. 10: Relative activity at of PtUGT1 WT, mutant 87, mutant 88 and mutant 90 in the presence of 15% organic solvent (either acetone, acetonitrile or isopropanol), in respect to the activity without organic solvents.

FIG. 11: Reaction chromatograms showing the absence or presence of product DCP-glucoside in a reaction using 4 mM DCP and either PtUGT1 WT or mutant 87 enzyme. The reaction with WT enzyme does not show any product, presumably due to low chemostability towards DCP. The reaction with mutant 87 shows a clear product peak, confirming the enhanced chemo-stability of this variant.

FIG. 12: Kinetics of indican synthesis using 100 mM indoxyl-acetate as substrate, 2U of Esterase from Bacillus subtilis (Sigma Aldrich), and different concentrations of PtUGT1/SuSy, at a constant molar ratio of 1:5 (50 μg, 20 μg, 10 μg, 5 μg for PtUGT1 WT or Mut 87; and 432.5 μg, 173 μg, 86.7 μg, 43.3 μg for SuSy). The results clearly shows that higher concentrations of Mut 87 reached higher concentrations of indican, while the reactions using PtUGT1 WT did not produce any indican, presumably due to chemical inactivation of the enzyme at high substrate concentration.

FIG. 13: Discs of 20 square centimeters of ready-to-dye denims (radius 1.784 cm, diameter 3.57 cm, weight 802+/−2 mg) are dyed in 3 ml of water at pH 9 with various amounts of indican (indicated on the picture) and 1 mg of Rye β-glucosidase 1. Discs are turned over every 5 min at room temperature for 15 min, and left for 1 h at room temperature before being washed with water and soap and dried overnight at room temperature. Pictures are taken four days after dyeing and after multiple washing/drying cycles. (A) Front side of samples; (B) Back side of samples.

FIG. 14: Indoxyl derivatives acceptors of PtUGT1 WT and mutant variants.

FIG. 15: Photo of color development in tubes comprising 6-Bromo-Indoxyl after 90 minutes incubation at 30° C. Tubes from left to right: Tube 1: (−) Control without UGT, Tube 2: (−) Control without UDP-Glc, Tube 3: (+) Control with PtUGT1 WT, Tube 4: With PtUGT1 Mut 87, Tube 5: With PtUGT1 Mut 88, Tube 6: With PtUGT1 Mut 90.

FIG. 16: Photo of color development in tubes comprising 5-Bromo-4-chloro-indoxyl after 60 minutes incubation at 30° C. From left to right (A) Tube 1: (−) Control without UGT, Tube 2: With PtUGT1 WT, Tube 3: With Mut 87, Tube 4: With Mut 88. (B) Tube 1: (−) Control without UGT, Tube 2: With Mut 90.

FIG. 17: Difference in the melting temperatures of prior art UGT enzymes compared to PtUGT1 WT.

FIG. 18: Chemostability. Reaction chromatograms showing the absence or presence of product DCP-glucoside in a reaction using 4 mM DCP and (a) PtUGT1 WT, (b) PtUGT2, (c) PtIGS, or (d) mutant 87 enzyme.

### ABBREVIATIONS, TERMS, AND DEFINITIONS:

Amino acid sequence identity: The term “sequence identity” as used herein, indicates a quantitative measure of the degree of similarity between two amino acid sequences of essentially equal length. The two sequences to be compared must be aligned to give a best possible fit, by means of the insertion of gaps or alternatively, truncation at the ends of the protein sequences. The sequence identity can be calculated as ((Nref−Ndif) 100)/(Nref), wherein Ndif is the total number of non-identical residues in the two sequences when aligned and wherein Nref is the number of residues in one of the sequences. Sequence identity calculations are preferably automated using the BLAST program e.g. the BLASTP program (Pearson W. R and D. J. Lipman (1988)) (www.ncbi.nlm.nih.gov/cgi-bin/BLAST). Sequence alignment may be performed using program MAFFT24 (Multiple Alignment using Fast Fourier Transform; Katoh et al 2019) using default parameters (SCORING MATRIX: blosum62, gap opening penalty: 1.53, gap extension penalty 0.123).

Preferably, the numbers of substitutions, insertions, additions or deletions of one or more amino acid residues in the polypeptide as compared to its comparator polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group 1: Glycine, Alanine, Valine, Leucine, Isoleucine; group 2: Serine, Cysteine, Selenocysteine, Threonine, Methionine; group 3: Proline; group 4: Phenylalanine, Tyrosine, Tryptophan; Group 5: Aspartate, Glutamate, Asparagine, Glutamine; Group 6: Histidine. Lysine, Arginine.

Melting temperature (Tm (° C.)) of a protein, as used herein, defines the temperature (Tm) at which both the folded and unfolded states are equally populated at equilibrium (assuming two-state protein folding), which is the denaturation midpoint of the protein, and is measured by using a thermal shift assay, such as the Protein Thermal Shift Dye Kit (ThermoFisher Scientific) and a qPCR QuantStudio5 machine-see examples section.

Half/shelf-life times are defined as the amount of time that an enzyme can be pre-incubated at a defined temperature having as a result a 50% residual activity compared with the activity without the pre-incubation.

Indoxyl compound is herein defined as indoxyl, thioindoxyl, and any indoxyl or thioindoxyl derivative having an unprotected (reactive) thio or hydroxyl group in position 3. Indoxyl derivatives may comprise halogen substitution(s) on the ring structure. Examples of indoxyl derivatives include, but are not limited to: 6-Bromo-indoxyl, 5-Bromo-4-chloro-indoxyl, 6-Chloro-indoxyl, 5-bromo-indoxyl, 5-bromo-6-chloro-indoxyl, Thioindoxyl, 5-bromo-7-bromo-indoxyl.

Reactive group is herein defined as a chemical group that can be glycosylated by a glycosyltransferase enzyme.

Free oxygen is herein defined as molecular oxygen or dioxygen.

Dye precursor is herein defined as a compound that can give rise to dyed material upon one or more chemical transformations.

Nucleotide sugar is herein defined as a molecule in which a sugar is bound to a nucleotide via a glycosidic bond; wherein the sugar is a monosaccharide, such as glucose, rhamnose, xylose, arabinose. Nucleotide sugars act as glycosyl donors in glycosylation reactions; those reactions are catalyzed by glycosyltransferases.

Mutant enzyme (or enzyme variant) is an enzyme which compared to the wild type enzyme comprises one of more amino acid substitutions.

### DETAILED DESCRIPTION OF THE INVENTION

The present invention provides improved glycosyltransferases.

As mentioned above, glycosyltransferases can be used in a variety of applications to attach a sugar molecule to different compounds, thereby enhancing their solubility, and decreasing volatility and potentially toxicity.

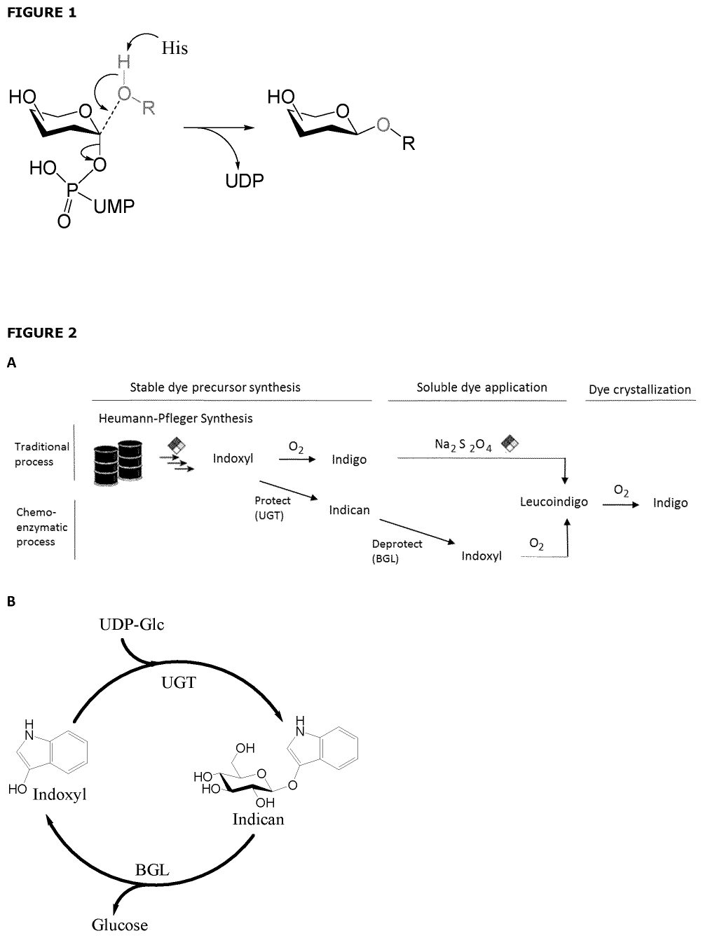

Specifically, UDP-dependent glycosyltransferase (UGT) is a superfamily of enzymes that catalyze glucosidation and help to transfer glycosyl from UDP-glycosyl donor to a variety of compounds. The enzymatic reaction is proposed to occur by deprotonation of the acceptor hydroxyl group by a highly conserved histidine residue in the UGT active site. The activated acceptor RO⁻ subsequently performs a nucleophilic attack at the C1 of the sugar donor to form a glycosidic bond (FIG. 1).

UGTs glycosylate many different chemicals, including indoxyl (indigo dye precursor). In particular, UGT enzyme variants can be applied as a green biotech alternative to current industrial processes for blue denim production (FIG. 2).

Blue denim is traditionally dyed with chemically synthesized indigo under harsh environmentally challenging conditions. As a final step in the synthesis, indigo forms spontaneously from indoxyl by oxidation by air, but for use in dying, indigo further needs to be solubilized with a strong reducing agent (e.g. Na₂S₂O₄), which is likewise environmentally challenging.

The improved, hyperstable glycosyltransferase enzyme variants described herein can be added to the current industrial process, thereby eliminating ‘dirty chemistry’ steps in blue denim dyeing. Specifically, the hydroxyl group of chemically synthesized indoxyl may be glycosylated by glycosyltransferase, thereby protecting the reactive functional group and generating the stable soluble (colorless) indican molecule. Indican may then later be hydrolyzed by beta-glucosidase (BGL) back to indoxyl which can then spontaneously oxidize to form blue indigo directly on the fabric. The invention thereby provides a “greener” alternative to the present industrial process, by providing an alternative solution to the final steps of the indigo dying process, whereby the use of the harsh strong reducing agent is avoided.

The application is equally applicable to similar indoxyl dye-compounds.

### I. An Improved UGT Enzyme

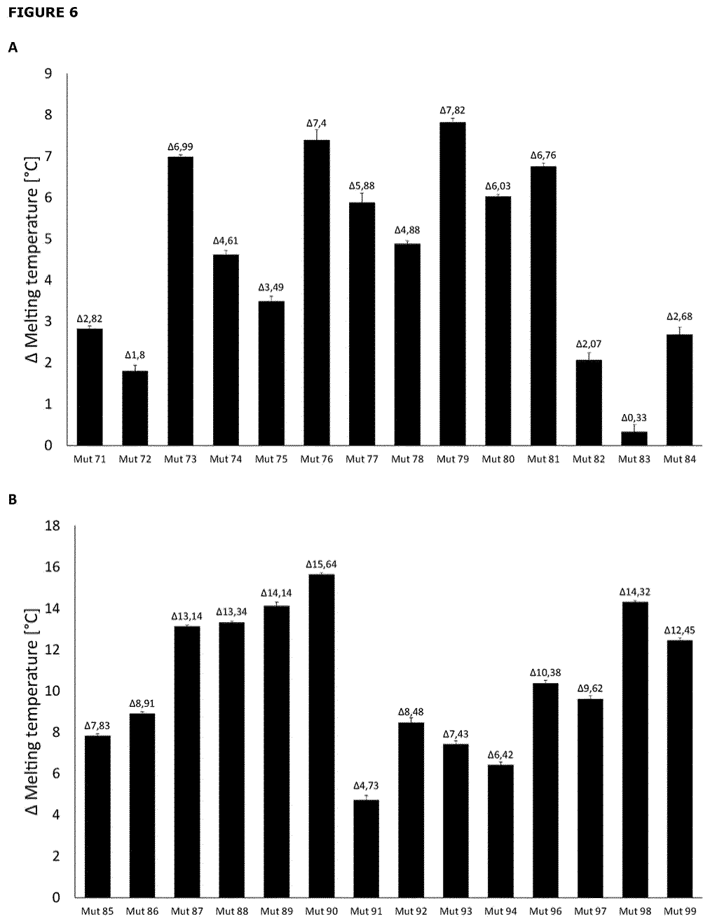

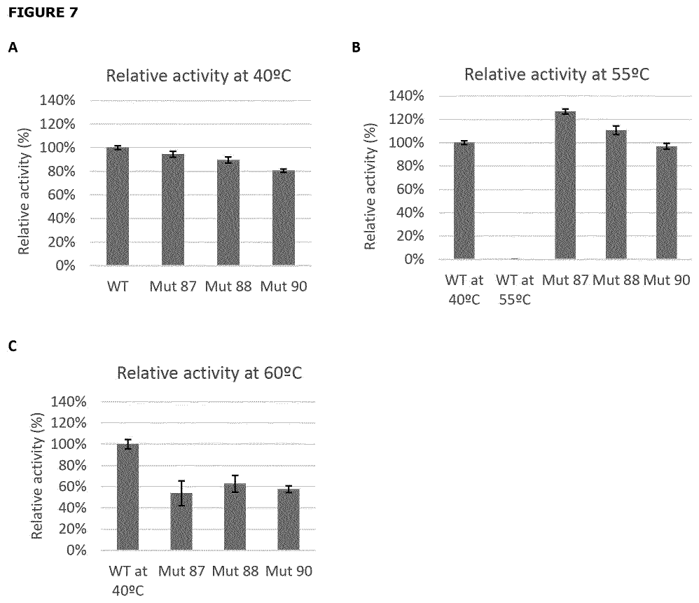

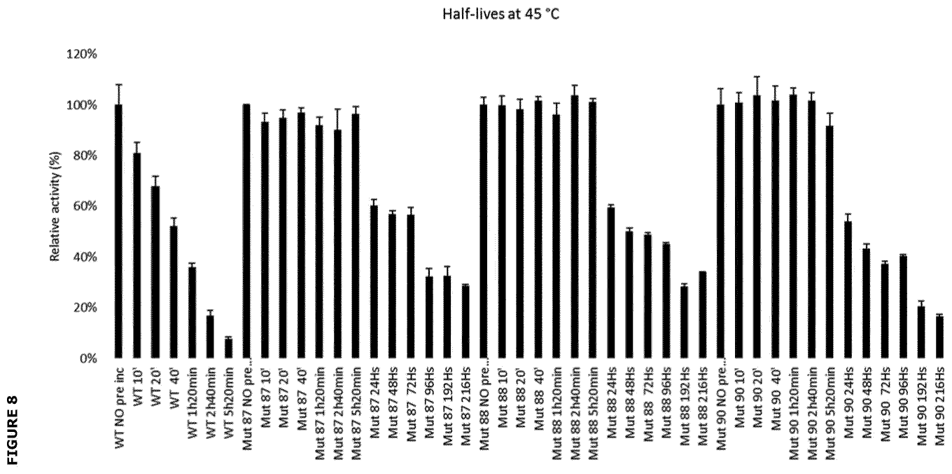

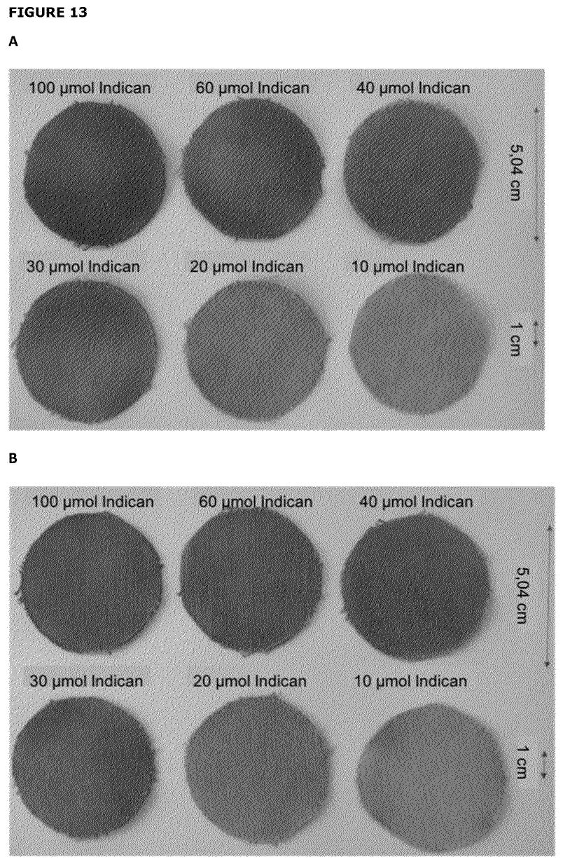

In one aspect, the present invention provides an improved glycosyltransferase mutant enzyme which has improved functional properties relative to the parent (wild type) enzyme form which the mutant was derived. Specifically, the glycosyltransferase mutant enzymes of the present invention are derived from PtUGT1 (SEQ ID NO 2), and have the following properties:
increased melting temperature (FIG. 6)
comparable activity at 40° C. (FIG. 7A)
activity at 55° C. and even 60° C., while the wildtype enzyme has no activity at those temperatures (FIGS. 7B and 7C).
increased half-life, by at least 216× for Mut 87, 144× for Mut 88, and 72× for Mut 90 at 45° C. (FIG. 8)
increased tolerance to different organic solvents (FIG. 10)
can stabilize different indoxyl compounds by formation of glycosylated soluble dye-precursors (FIGS. 12, 15 and 16), and thus be used in dyeing applications such as denim dyeing (FIG. 13).

As part of natural processing of proteins in microbial organisms, the leading methionine amino acid residue is naturally removed and hence is not part of the final mature protein. Therefore, reference to specific amino acid positions in the amino acid sequence of the wild type PtUGT1 enzyme is preferably done using the amino acid sequence without the leading methionine. In the present application, SEQ ID NO. 2 and SEQ ID NO. 195 both represent the amino acid sequence of wild type PtUGT1, the only difference being that SEQ ID NO. 2 does not comprise the leading methionine residue, while SEQ ID NO. 195 comprises the leading methionine residue. The mutant glycosyltransferase enzyme of the present invention possesses glycosyltransferase activity (enzyme classification EC: 2.4.1.-) for glycosylating a selected compound, said compound having a reactive group. The mutant enzyme has at least 75% sequence identity to wild type UDP-dependent glycosyltransferase (PtUGT1, SEQ ID NO. 2) from Polygonum tinctorium/Persicaria tinctoria, but comprises one or more specific mutations relative to the sequence of PtUGT1. Specifically, the mutant comprises (i) one or more amino acid residue substitutions selected from: E75P, Q86K, S110V, I188L, G222D, G296L, V297G, F381V, T388A, S413K and G430K relative to the amino acid sequence of PtUGT1, and/or (ii) amino acid residue substitutions T388C and A399C relative to the amino acid sequence of PtUGT1.

In one embodiment, the mutant glycosyltransferase enzyme of the present invention has glycosyltransferase activity, and the amino acid sequence of said enzyme comprises one or more of the amino acid substitutions disclosed above, relative to PtUGT1 parent (wild type) enzyme, and further has at least 75% sequence identity to PtUGT1 (SEQ ID NO.: 2), such as at least 76, 77, 78, 79, 80, 81, 82, 83, 84, 85, 86, 87, 88, 89, 90, 91, 92, 93, 94, 95, 96, 97% sequence identity to PtUGT1 (SEQ ID NO.: 2).

In one embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, 97% sequence identity to SEQ ID NO. 2, and wherein said amino acid sequence comprises (i) one or more amino acid residue substitutions selected from: E75P, Q86K, S110V, I188L, G222D, G296L, V297G, F381V, T388A, S413K and G430K, and/or (ii) amino acid residue substitutions T388C and A399C, with respect to SEQ ID NO. 2.

In one embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, 97% sequence identity to SEQ ID NO. 2, and wherein said amino acid sequence comprises amino acid residue substitutions E75P, Q86K, S110V, I188L, G222D, G296L, V297G, S413K, and G430K with respect to SEQ ID NO. 2.

In one embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, 97% sequence identity to SEQ ID NO. 2, and wherein said amino acid sequence comprises (i) amino acid residue substitutions E75P, Q86K, S110V, I188L, G222D, G296L, V297G, S413K, and G430K with respect to SEQ ID NO. 2, and (iia) one or more amino acid residue substitutions selected from F381V and T388A with respect to SEQ ID NO. 2.

In one embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, 97% sequence identity to SEQ ID NO. 2, and wherein said amino acid sequences comprises (i) amino acid residue substitutions E75P, Q86K, S110V, I188L, G222D, G296L, V297G, S413K, and G430K with respect to SEQ ID NO. 2, and (iib) amino acid residue substitutions T388C and A399C with respect to SEQ ID NO. 2.

In one embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, or 97% sequence identity to SEQ ID NO. 2, and wherein said amino acid sequence comprises (i) amino acid residue substitutions E75P, Q86K, S110V, I188L, G222D, G296L, V297G, S413K, and G430K with respect to SEQ ID NO. 2, and (iia) one or more amino acid residue substitutions selected from F381V and T388A, and (iib) amino acid residue substitutions T388C and A399C with respect to SEQ ID NO. 2.

In one preferred embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, or 97% sequence identity to SEQ ID NO. 2, and wherein said amino acid sequence comprises amino acid residue substitutions E75P, Q86K, S110V, I188L, G222D, G296L, V297G, S413K, G430K, and T388A with respect to SEQ ID NO. 2.

In one most preferred embodiment, the present invention provides a polypeptide having glycosyltransferase activity (enzyme classification EC: 2.4.1.-), wherein the amino acid sequence of said polypeptide is SEQ ID NO. 4.

In the present application, SEQ ID NO. 4 and SEQ ID NO. 196 both represent the amino acid sequence of Mut97, the only difference being that SEQ ID NO. 4 comprises the leading methionine residue, while SEQ ID NO. 196 does not comprise the leading methionine residue.

In another preferred embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, or 97% sequence identity to SEQ ID NO. 2, and wherein said amino acid sequence comprises amino acid residue substitutions E75P, Q86K, S110V, I188L, G222D, G296L, V297G, S413K, G430K, F381V and T388A with respect to SEQ ID NO. 2.

In another most preferred embodiment, the present invention provides a polypeptide having glycosyltransferase activity (enzyme classification EC: 2.4.1.-), wherein the amino acid sequence of said polypeptide is SEQ ID NO. 6.

In the present application, SEQ ID NO. 6 and SEQ ID NO. 197 both represent the amino acid sequence of Mut88, the only difference being that SEQ ID NO. 6 comprises the leading methionine residue, while SEQ ID NO. 197 does not comprise the leading methionine residue.

In another preferred embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, or 97% sequence identity to SEQ ID NO. 2, and wherein said amino acid sequence comprises amino acid residue substitutions E75P, Q86K, S110V, I188L, G222D, G296L, V297G, S413K, G430K, F381V, T388C, and A399C with respect to SEQ ID NO. 2.

In another most preferred embodiment, the present invention provides a polypeptide having glycosyltransferase activity (enzyme classification EC: 2.4.1.-), wherein the amino acid sequence of said polypeptide is SEQ ID NO. 8.

In the present application, SEQ ID NO. 8 and SEQ ID NO. 198 both represent the amino acid sequence of Mut90, the only difference being that SEQ ID NO. 8 comprises the leading methionine residue, while SEQ ID NO. 198 does not comprise the leading methionine residue. In one preferred embodiment, the polypeptide of the invention has UPD-dependent glycosyltransferase activity. In a further preferred embodiment, the polypeptide of the invention has indoxyl-UDPG glucosyltransferase activity (enzyme classification EC: 2.4.1.220) The mutant glycosyltransferase enzyme of the present invention possesses glycosyltransferase activity (enzyme classification EC: 2.4.1.-) for glycosylating a selected compound, said compound having a reactive group. The mutant enzyme has at least 75% sequence identity to wild type UDP-dependent glycosyltransferase (PtUGT1, SEQ ID NO. 195) from Polygonum tinctorium/Persicaria tinctoria, but comprises one or more specific mutations relative to SEQ ID NO. 195. Specifically, the mutant comprises (i) one or more amino acid residue substitutions selected from: E76P, Q87K, S111V, I189L, G223D, G297L, V298G, F381V, T389A, S414K and G431K relative to SEQ ID NO. 195, and/or (ii) amino acid residue substitutions T389C and A400C relative to SEQ ID NO. 195.

In one embodiment, the mutant glycosyltransferase enzyme of the present invention has glycosyltransferase activity, and the amino acid sequence of said enzyme comprises one or more of the amino acid substitutions disclosed above, relative to SEQ ID NO. 195, and further has at least 75% sequence identity to SEQ ID NO. 195, such as at least 76, 77, 78, 79, 80, 81, 82, 83, 84, 85, 86, 87, 88, 89, 90, 91, 92, 93, 94, 95, 96, 97% sequence identity to SEQ ID NO. 195.

In one embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, 97% sequence identity to SEQ ID NO. 195, and wherein said amino acid sequence comprises (i) one or more amino acid residue substitutions selected from: E76P, Q87K, S111V, I189L, G223D, G297L, V298G, F3812V, T389A, S414K and G431K, and/or (ii) amino acid residue substitutions T389C and A400C, with respect to SEQ ID NO. 195.

In one embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, 97% sequence identity to SEQ ID NO. 195, and wherein said amino acid sequence comprises amino acid residue substitutions E76P, Q87K, S111V, I189L, G223D, G297L, V298G, S414K, and G431K with respect to SEQ ID NO. 195.

In one embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, 97% sequence identity to SEQ ID NO. 195, and wherein said amino acid sequence comprises (i) amino acid residue substitutions E76P, Q87K, S111V, I189L, G223D, G297L, V298G, S414K, and G431K with respect to SEQ ID NO 195, and (iia) one or more amino acid residue substitutions selected from F382V and T389A with respect to SEQ ID NO. 195.

In one embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, 97% sequence identity to SEQ ID NO. 195, and wherein said amino acid sequences comprises (i) amino acid residue substitutions E76P, Q87K, S111V, I189L, G223D, G297L, V298G, S414K, and G431K with respect to SEQ ID NO 195, and (iib) amino acid residue substitutions T389C and A400C with respect to SEQ ID NO. 195.

In one embodiment, the present invention provides a polypeptide p having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, or 97% sequence identity to SEQ ID NO. 195, and wherein said amino acid sequence comprises (i) amino acid residue substitutions E76P, Q87K, S111V, I189L, G223D, G297L, V298G, S414K, and G431K with respect to SEQ ID NO 195, and (iia) one or more amino acid residue substitutions selected from F382V and T389A, and (iib) amino acid residue substitutions T389C and A400C with respect to SEQ ID NO. 195.

In one preferred embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, or 97% sequence identity to SEQ ID NO. 195, and wherein said amino acid sequence comprises amino acid residue substitutions E76P, Q87K, S111V, I189L, G223D, G297L, V298G, S414K, G431K, and T389A with respect to SEQ ID NO. 195.

In one most preferred embodiment, the present invention provides a polypeptide having glycosyltransferase activity (enzyme classification EC: 2.4.1.-), wherein the amino acid sequence of said polypeptide is SEQ ID NO. 196.

In the present application, SEQ ID NO. 4 and SEQ ID NO. 196 both represent the amino acid sequence of Mut97, the only difference being that SEQ ID NO. 4 comprises the leading methionine residue, while SEQ ID NO. 196 does not comprise the leading methionine residue.

In another preferred embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, or 97% sequence identity to SEQ ID NO. 195, and wherein said amino acid sequence comprises amino acid residue substitutions E76P, Q87K, S111V, I189L, G223D, G297L, V298G, S414K, G431K, F382V and T389A with respect to SEQ ID NO. 195.

In another most preferred embodiment, the present invention provides a polypeptide having glycosyltransferase activity (enzyme classification EC: 2.4.1.-), wherein the amino acid sequence of said polypeptide is SEQ ID NO. 197.

In the present application, SEQ ID NO. 6 and SEQ ID NO. 197 both represent the amino acid sequence of Mut88, the only difference being that SEQ ID NO. 6 comprises the leading methionine residue, while SEQ ID NO. 197 does not comprise the leading methionine residue.

In another preferred embodiment, the present invention provides a polypeptide having glycosyltransferase activity (enzyme classification EC: 2.4.1.-), wherein the amino acid sequence of said polypeptide has at least 75, 76, 77, 78, 79, 80, 81, 82, 83, 84, 85, 86, 87, 88, 89, 90, 91, 92, 93, 94, 95, 96, or 97% sequence identity to SEQ ID NO. 195, and wherein said amino acid sequence comprises amino acid residue substitutions E76P, Q87K, S111V, I189L, G223D, G297L, V298G, S414K, G431K, F382V, T389C, and A400C with respect to SEQ ID NO. 195.

In another most preferred embodiment, the present invention provides a polypeptide having glycosyltransferase activity (enzyme classification EC: 2.4.1.-), wherein the amino acid sequence of said polypeptide is SEQ ID NO. 198.

In the present application, SEQ ID NO. 8 and SEQ ID NO. 198 both represent the amino acid sequence of Mut90, the only difference being that SEQ ID NO. 8 comprises the leading methionine residue, while SEQ ID NO. 198 does not comprise the leading methionine residue.

### II. A Composition Comprising the Mutant UGT Enzyme

In a second aspect, the present invention provides a composition comprising (i) a polypeptide as disclosed in section I having glycosyltransferase enzyme activity, (ii) a compound comprising a reactive group, and (iii) nucleotide sugar.

In some cases, the reactive group of the compound which is to be glycosylated may in the presence of oxygen react with the oxygen—such as in competition with the enzymatic glycosylation reaction. In such case, it may therefore be an advantage to provide an oxygen reduced, oxygen free, or substantially oxygen free environment for the glycosylation reaction to take place.

In one embodiment, the composition of the present invention is substantially oxygen free. In one embodiment, the composition of the present invention comprises less than 2% free oxygen, such as less than 1.5, 1, 0.5, or even less than 0.1% free oxygen and/or is maintained as a pressure less than 10, 9, 8, 7, 6, 5, 4, 3, 2 kPa or even less than 1 kPa, to reduce likelihood of oxidizing the reactive group of the compound.

In one embodiment, the reactive group of the compound in the composition is a hydroxyl group. In one embodiment, the compound comprising a reactive group is an indoxyl compound, and the composition is suitable for obtaining a stabilized dye precursor, as the indoxyl compound is glycosylated by the glycosyltransferase enzyme. In a preferred embodiment, the compound comprising a reactive group is selected from indoxyl, 6-bromo-indoxyl, 5-bromo-4-chloro-indoxyl, 6-Chloro-indoxyl, 5-bromo-indoxyl, 5-bromo-6-chloro-indoxyl, thioindoxyl, and 5-bromo-7-bromo-indoxyl. In one embodiment, the polypeptide as disclosed in section I having glycosyltransferase enzyme activity is a UPD-dependent glycosyltransferase, and the nucleotide sugar is a UPD-sugar, such as UDP-glucose, UPD-rhamnose, UPD-xylose, and UPD-arabinose. In a preferred embodiment, the nucleotide sugar of the composition is UDP-glucose. In a most preferred embodiment, the compound comprising a reactive group is indoxyl, which is converted to indican (a stable precursor of indigo) by the glycosyltransferase enzyme.

### III. A Kit of Parts

In a third aspect, the present invention provides a kit of parts comprising (i) a polypeptide as disclosed in section I having glycosyltransferase enzyme activity, and (ii) a polypeptide encoding a beta-glucosidase (BGL) (enzyme classification EC 3.2.1.21).

The kits of parts of the present invention comprises a glycosyltransferase enzyme as defined herein and a BGL enzyme as defined herein. Exemplified by their action on indoxyl (FIG. 2B), these enzymes catalyze separate reactions: Firstly the glycosyltransferase enzyme glycosylates and thereby stabilizes the indoxyl compound (i.e. forming indican), and then later at desired reaction conditions the BLG deglycosylates indican to regain the reactive indoxyl compound.

A person skilled in the art will be familiar with methods of providing the different enzymes for the kit of the present invention. Such enzymes may for example be microbially produced-such as recombinantly or by natural producers, or be synthesized. The enzymes may be provided in solution or dried form. The enzymes may be premixed or provided in separate containers.

### III.i Glycosyltransferase

For details pertaining to the polypeptide having glycosyltransferase enzyme activity of the kit of the invention, see section I of the present application.

### III.ii Beta-Glucosidase

Beta-glucosidase (BGL) catalyzes the cleavage of glycoside bonds and is in regard to the present invention applied to remove glucose from the glycosylated compound. Such removal of the (protecting) sugar molecule will convert the compound back to its reactive form. Where the compound is an indoxyl compound, this may in its reactive form spontaneously dimerize under aerobic conditions.

In one embodiment, the BGL is selected from the group of enzymes classified as EC 3.2.1.21.

In one embodiment, the amino acid sequence of the BGL is one having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO. 9, 10, or 11.

In one preferred embodiment, the amino acid sequence of the BGL is one having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO. 9.

### III.iii Nucleotide Sugar

The kit may further comprise a nucleotide sugar, which is required for the glycosylation reaction catalyzed by glycosyltransferase. In one embodiment the nucleotide sugar is a UPD-sugar, such as UDP-glucose, UPD-rhamnose, UPD-xylose, and UPD-arabinose. In a preferred embodiment, the nucleotide sugar is UDP-glucose. UDP-glucose may be provided directly as UDP-glucose or indirectly in the form of other sugars or sugar-containing molecules which are then converted into UDP-glucose. One example of such indirect providing of UPD-glucose is by providing sucrose along with UDP, which then by enzymatic catalysis (such as using Sucrose synthase (SuSy) EC 2.4.1.13) is converted to UDP-glucose—as illustrated in example 4.1. Another example of indirect providing of UPD-glucose is by using sucrose phosphorylase (converts sucrose and phosphate into glucose-1-P and fructose), glucose-1-phosphate uridylyltransferase (converts UTP and glucose-1-P into UDP-glucose and PPi); and further to regenerate the UTP from UDP, using acetate kinase which requires acetyl-P as substrate in equimolar amounts (converts Acetyl-P+UDP to UTP+acetate) (Lee et al 2004, Bruyn et al 2015).

The kit may further comprise buffers and other relevant reagents for either maintaining activity of the enzymes and/or for enhancing the effect of the enzymes.

Finally, the kit may further comprise an instruction manual providing specifics for each kit component and/or a description of a method of using the kit components.

### IV. Methods Involving the Improved UGT Enzyme

In a fourth aspect, different methods involving the mutant glycosyltransferase enzymes of the present invention are provided, wherein the glycosyltransferase enzyme catalyzes glycosylation of a compound of interest.

### IV.i A Method for Glycosylating a Compound

In a fourth aspect, the present invention provides a method for glycosylating a compound, comprising the steps of:
a. providing (i) a compound comprising a reactive group, (ii) a polypeptide of the present invention (as disclosed in section I) having glycosyltransferase enzyme activity, and (iii) a nucleotide sugar,
b. mixing the components provided in step (a),
c. letting the mixture react to obtain the glycosylated compound.

As disclosed herein, the mutant glycosyltransferase enzyme of the present invention possesses glycosyltransferase activity for glycosylating a selected compound comprising a reactive group.

In one embodiment, the compound comprising a reactive group is an indoxyl compound.

In a preferred embodiment, the indoxyl compound is selected from indoxyl, 6-bromo-indoxyl, 5-bromo-4-chloro-indoxyl, 6-Chloro-indoxyl, 5-bromo-indoxyl, 5-bromo-6-chloro-indoxyl, Thioindoxyl, and 5-bromo-7-bromo-indoxyl. In a most preferred embodiment, the compound comprising a reactive group is indoxyl.

Further, as disclosed previously, a nucleotide sugar must be present for the reaction to take place, but can be provided directly or indirectly as described in section III.iii. In one embodiment, the method of glycosylating a selected compound comprises providing UPD-glucose. In another embodiment in step (a) of the method, a sugar molecule is provided, which can be converted into a nucleotide sugar, preferably UDP-glucose, such as by enzymatic catalysis.

Reaction conditions of the method may depend on what the target compound for glycosylation is.

In some cases, the reactive group of the compound which is to be glycosylated may in the presence of oxygen react with the oxygen-such as in competition with the enzymatic glycosylation reaction. In such case, it may therefore be an advantage to provide an oxygen reduced, oxygen free, or substantially oxygen free environment for the glycosylation reaction to take place.

In one embodiment, step (b) in the method of glycosylating a compound is performed under conditions, where less than 2% free oxygen, such as less than 1.5, 1, 0.5, or even less than 0.1% free oxygen and/or is maintained as a pressure less than 10, 9, 8, 7, 6, 5, 4, 3, 2 kPa or even less than 1 kPa, to reduce likelihood of oxidizing the reactive group of the compound. In one embodiment, the reactive group of the target compound for glycosylation is a hydroxyl group.

In one embodiment, where the target compound for glycosylation is an indoxyl compound, the reaction preferably takes place at oxygen reduced, substantially oxygen free, or even anaerobic conditions to ensure the reactive indoxyl compound does not spontaneously dimerize. In one embodiment, where the target compound for glycosylation is an indoxyl compound, the reaction preferably takes place at conditions comprising less than 2% free oxygen, such as less than 2, 1.5, 1, 0.5, or even less than 0.1%, and/or decreased pressure such as less than 10, 9, 8, 7, 6, 5, 4, 3, 2 or even less than 1 kPa-to reduce likelihood of the reactive indoxyl compound spontaneously dimerizing, such as indoxyl dimerizing to form indigo.

The compound comprising a reactive group is preferably incubated with the glycosyltransferase enzyme at temperature and pH conditions optimal for the enzyme.

In one embodiment, the incubation temperature applied should be in the range 20-65° C., such as 20-60° C., such as 30-60° C., such as 40-55° C., preferably in the range 45-55° C., such as preferably around 50° C. In one embodiment, the incubation pH applied should be in the range pH 5-9, such as pH 5.5-8.5, such as preferably pH 6-8.

The enzymatic reaction may take place in buffered solution for stabilizing the enzymes, as a person skilled in the art would recognize and routinely optimize.

The glycosylated compounds produced by the method of the present invention may be detected by HLPC-UV, LC-MS, NMR, or similar equipment as recognized by a person skilled in the art.

IV.ii. A Method for Producing a Soluble Dye Precursor

As disclosed previously, indoxyl compounds may under aerobic conditions dimerize and form colored compounds, which may be used as dyes, such as for dyeing fabrics or other products. These dimerized colored compounds are insoluble in an aqueous solution. Glycosylation of the indoxyl compound will stabilize the compound, prevent dimerization, and thereby provide a soluble dye precursor.

In one embodiment, a method for producing a soluble dye precursor is provided, comprising the steps:
a. providing (i) an indoxyl compound, (ii) a polypeptide of the present invention (as disclosed in section I) having glycosyltransferase enzyme activity, and (iii) a nucleotide sugar,
b. mixing the components provided in step (a), preferably at reaction conditions wherein less than 2% free oxygen is present,
c. letting the mixture react to obtain the glycosylated soluble indoxyl dye precursor.

Reaction conditions specified in section IV.i equally apply to this method for producing a soluble dye precursor.

IV.iii. A Method for Dyeing a Product, such as Yarn or Textile

The mutant glycosyltransferase enzyme of the present invention is particularly useful in an enzyme-catalyzed method for dyeing products, such as yarn or textiles, thus proving an alternative to the current chemical process.

In one embodiment, the method for producing a soluble dye precursor disclosed in section IV.ii additionally comprises dying a product of instead, by further comprising the steps:
d. mixing the glycosylated soluble indoxyl dye precursor with a product of interest and a polypeptide having a beta-glucosidase enzyme activity (enzyme classification EC 3.2.1.21), at reaction conditions wherein free oxygen is present, to obtain a dyed product.

In one embodiment, the present invention provides a method for dying a product is provided, comprising the steps of
a. providing (i) an indoxyl compound, (ii) a polypeptide as disclosed in section I having glycosyltransferase enzyme activity, (iii) a nucleotide sugar, and (iv) a polypeptide having a beta-glucosidase enzyme activity,
b. mixing components (i), (ii), and (iii) provided in step (a), preferably at reaction conditions wherein less than 2% free oxygen is present,
c. letting the mixture react to obtain a soluble glycosylated indoxyl dye-precursor,
d. mixing said dye precursor with said product and said beta-glucosidase at reaction conditions wherein free oxygen is present, to obtain a dyed product.

In one embodiment, the product intended for dying by the methods disclosed herein, may be selected from yarn, textile, fabrics, and similar products. In a preferred embodiment, the product is a yarn or a textile.

In regard to steps (a), (b), and (c), reaction conditions specified in section IV.i equally apply to this method for dyeing a textile. In regard to step (d), the product intended for dyeing is mixed with the dye precursor, and the method further comprises the step of mixing/adding a beta-glucosidase enzyme to then de-glycosylate the indoxyl compound. Such beta-glucosidase enzymes are described in section III.ii. Preferably, this part of the method takes place in aerobic conditions, whereby the indoxyl compound spontaneously dimerize and form a colored dye.

In a preferred embodiment, the indoxyl compound in the method for dyeing a product is indoxyl, the dye precursor is indican, and the final dyed product is dyed by indigo. In a much preferred embodiment, the final dyed product is a textile.

### V. Use of the Improved UGT Enzyme

In a fifth aspect, the present invention discloses the use of a polypeptide having glycosyltransferase enzyme activity as disclosed in section I, in glycosylating a compound, wherein said compound comprises a reactive hydroxyl group.

In a preferred embodiment, the compound is an indoxyl compound, and the glycosylated compound is used as a dye-precursor in the process of dying textiles.

### VI. Advantages and Commercial Application

As discussed previously, and further evidenced in the below examples, the glycosyltransferase enzymes of the present invention are improved compared to the wild type enzyme in several aspects, including melting temperature, half-life, solvent tolerance and chemo-stability. Such improvements are highly relevant commercially. The enzymes are particularly suited as a greener alternative to current fabric and textile dyeing processes, but may be used in many other

### EXAMPLES

In the following, several different mutant UGT polypeptides are studied and characterized. Table 2, 3 and 4 provide an overview of these mutant enzymes and their amino acid mutations relative to the PtUGT1 wild type. The mutant enzymes are in this application generally referred to by their “mutant number”.

### General Methodology

### Mutant Design

Variants of wild type PtUGT1 (SEQ ID NO. 2) were constructed using the original expression vector pTMH307 (SEQ ID NO. 12) as template (Hsu et. al 2018; GenBank accession No. MF688772). The mutations were introduced by PCR using USER cloning (NEB). The primers used for mutagenesis are shown in table 1. All constructs were verified by DNA sequencing service (Eurofins) before transformation into chemically competent E. coli BL21 Star (DE3) (ThermoFisher Scientific) following manufacturer recommendations.

TABLE 1
Primers for making amino acid mutations in PtUGT1

| Mutation | Primer forward | Primer reverse |
|---|---|---|
| P14C | ATG CCA CGU CAT AAT CGT GCC | ACG TGG CAU GGT GGA GCG |
| | CTC CGC CGG C (SEQ ID NO. 15) | GTG GTT GGT (SEQ ID NO. 16) |
| A117C | ACC TTT UCG CCA CTG ATG CAA | AAA AGG UCG ACG ACG AGG |
| | TCG ACG T (SEQ ID NO. 17) | GCG CAG ACG CGG CGG CCG |
| | | GAG (SEQ ID NO. 18) |
| S21C | ATC GTG CCC UGC GCC GGC ATG | AGG GCA CGA UTA TGA CGT |
| | GGC CAC CTC AT (SEQ ID NO. 19) | GCG GTG GTG G (SEQ ID NO. |
| | | 20) |
| D122C | ACT GAT GCA AUC GAC GTC GCC | ATT GCA TCA GUG GCG AAA |
| | CTT GAG CTC (SEQ ID NO. 21) | AGG CAG ACG ACG AGG GCG |
| | | GCG (SEQ ID NO. 22) |
| V76C/R97C | ACG CCC AAA UCG AGA CTC TCA | ATT TGG GCG UCG GAG GGG |
| | TGT CCC TCA TGG TTG TCT GCT | GCG TCG GAG AGG TCG CAC |
| | CCC TCC CCT CGC TCC GC (SEQ | TCG GGG AGG AAG GAG |
| | ID NO. 23) | GT (SEQ ID NO. 24) |
| L119C/A132C | CC TTT UCG CCA CTG ATG CAA | AAA AGG UCG ACG ACG CAG |
| | TCG ACG TCT GCC TTG AGC TCG | GCG GCG ACG CGG CGG C (SEQ |
| | GCA TCC GCC CTT T (SEQ ID NO. | ID NO. 26) |
| | 25) | |
| V121C/A125C | ACC TTT UCT GCA CTG ATG CAA | AAA AGG UCG CAG ACG AGG |
| | TCG ACG TCG CCC (SEQ ID NO. | GCG GCG ACG CG (SEQ ID NO. |
| | 27) | 28) |
| T126C | ATG CAA UCG ACG TCG CCC TTG | ATT GCA UCG CAG GCG AAA |
| | AGC T (SEQ ID NO. 29) | AGG TCG ACG AC (SEQ ID NO. |
| | | 30) |
| R208C | AAG TGC TAU AAA TTG GCC GAG | ATA GCA CTU GGA GTG GTG |
| | GGT GTT ATC GTA (SEQ ID NO. | GAG GAG CCA CTT (SEQ ID NO. |
| | 31) | 32) |
| A132C/I137C | AGC TCG GCU GCC GCC CTT TCA | AGC CGA GCU CAA GGC AGA |
| | TCT TCT TCC CCT CC (SEQ ID NO. | CGT CGA TTG CAT CAG T (SEQ |
| | 33) | ID NO. 34) |
| A132C/P139C | AGC TCG GCA UCC GCT GCT TCA | ATG CCG AGC UCA AGG CAG |
| | TCT TCT TCC CCT CCA CCG CC | ACG TCG ATT GCA TCA GT (SEQ |
| | (SEQ ID NO. 35) | ID NO. 36) |
| A147C | ACC TGC AUG ACC CTC TCC TTC | ATG CAG GUG GAG GGG AAG |
| | TTC CT (SEQ ID NO. 37) | AAG ATG AAA G (SEQ ID NO. 38) |
| P227C | AGG GGG GAU GCA TCA GGG AGC | ATC CCC CCU CCA AAC CCT CGA |
| | TTT TGC ACC CC (SEQ ID NO. 39) | AGC TAT (SEQ ID NO. 40) |
| P173C/P181C | ATC CCC GGG UGT ATT TGC GTC | ACC CGG GGA UCT GAA CGC |
| | CAC GGC AAG GAT TTG ATC GAC | AGT CGG ACA GCT CGG CAA A |
| | (SEQ ID NO. 41) | (SEQ ID NO. 42) |
| I176C | AGT GCC CCG GGU GTA TTC CGG | ACC CGG GGC ACU GAA CCG |
| | TCC ACG GCA AGG ATT (SEQ ID | GGT CGG ACA GCT (SEQ ID NO. |
| | NO. 43) | 44) |
| D186C | AGT GCT UGA TCG ACC CGG TTC | AAG CAC UTG CCG TGG ACC |
| | AGG ATA GGA (SEQ ID NO. 45) | GGA ATA C (SEQ ID NO. 46) |
| K396C | AAT GCA UGA ACG CTG TTA TGC | ATG CAT UGC TCT GCA TAG AGG |
| | TAA CCG AGG G (SEQ ID NO. 47) | GGC CAT GT (SEQ ID NO. 48) |
| P190C/A198C | AGA ACG ACU GCT ACA AGT GGC | AGT CGT TCU TCC TAT CCT GAA |
| | TCC TCC ACC ACT CC (SEQ ID NO. | CGC AGT CGA TCA AAT CCT TGC |
| | 49) | C (SEQ ID NO. 50) |
| D193C/N196C | AGG AAG UGC GAC GCC TAC AAG | ACT TCC UGC ACT GAA CCG GGT |
| | TGG CTC CTC C (SEQ ID NO. 51) | CGA TCA A (SEQ ID NO. 52) |
| A259C/L264C | AGT GCT GCA AGU GGT TGG ACC | ACT TGC AGC ACU CAG GCC |
| | AGC AGC CAC GTG GAT (SEQ ID | GGC AAG CTG CCC CCT TCT CGC |
| | NO. 53) | A (SEQ ID NO. 54) |
| P271C/S274C | AGT GCC GUG GAT GCG TCC TAT | ACG GCA CUG CTG GTC CAA |
| | TCG TGA ATT TCG GGA GT (SEQ | CCA CTT CA (SEQ ID NO. 56) |
| | ID NO. 55) | |
| G273C | ACG TTG CUC CGT CCT ATT CGT | AGC AAC GUG GCT GCT GGT |
| | GAA TT (SEQ ID NO. 57) | CCA ACC ACT T (SEQ ID NO. 58) |
| G365C | ACG TGC GGG UTC TTG ACG CAT | ACC CGC ACG UCG ACT CAT |
| | TGT GGG TGG AAT T (SEQ ID NO. | GGC TTA AGA C (SEQ ID NO. 60) |
| | 59) | |
| V278C | ATT CTG CAA UTT CGG GAG TGG | ATT GCA GAA UAG GAC GGA |
| | TGG GGT C (SEQ ID NO. 61) | TCC ACG TGG C (SEQ ID NO. 62) |
| W307C | ATG CGT GGU TAG GCC TCC AAA | ACC ACG CAU AGG AAC CTC TGC |
| | CGA CGG CAT TG (SEQ ID NO. 63) | TGG CTG (SEQ ID NO. 64) |
| L286C/Q290C | AGT ACG GAG UGC CAG AAC GAG | ACT CCG TAC UGC AGA CCC CAC |
| | CTT GCA GGT GTG C (SEQ ID NO. | CAC TCC CGA AA (SEQ ID NO. |
| | 65) | 66) |
| G337C/E340C | AGT GCT TCU TGT GCC AGA CCG | AGA AGC ACU CGG GCA GGA |
| | CGG GCA GGG GTT T (SEQ ID NO. | GTT TCA ACG (SEQ ID NO. 68) |
| | 67) | |
| T342C/G346C | AGG TGC UTG GTC TTG CCA ATG | AGC ACC UGC CCG CGC ACT |
| | TGG GCC CC (SEQ ID NO. 69) | GCT CCA AGA ACC CCT C (SEQ |
| | | ID NO. 70) |
| L368C/I387C | AGA GCG UGT TCC ATG GGG TAC | ACG CTC UCC AGT GTT GAA TTC |
| | CAC TAT GCA CAT GGC CCC TCT | CAC CCA CAA TGC GTG CAG AAC |
| | ATG CAG AGC AA (SEQ ID NO. 71) | CCG CCC GTC GAC TCA (SEQ ID |
| | | NO. 72) |
| T388C/A399C | AGC AAA AGA UGA ACT GCG TTA | ATC TTT TGC UCT GCA TAG AGG |
| | TGC TAA CCG AGG GCC TGA GG | GGC CAG CAA ATT AGT GGT ACC |
| | (SEQ ID NO. 73) | CCA TGG AAC A (SEQ ID NO. 74) |
| P412C/E424C | ATG GAA UCA TCC GAG GTG CTT | ATT CCA UCC TTA CCC ACT GAG |
| | GCA TCG CAC GAG TTA TAG GGG | CAT CTG AGT CCC ACC CTC AG |
| | AGT TG (SEQ ID NO. 75) | (SEQ ID NO. 76) |
| V455C/S462C | AGC AAA GAU GGA TCA TGC ACT | ATC TTT GCU CAA GCA AGC AGA |
| | CGA GCT CTT GAA GAG GTT GCA | AGC CGC ACG CTT (SEQ ID NO. |
| | (SEQ ID NO. 77) | 78) |
| S457C/G460C | AAA GAT UGC TCA TCT ACT CGA | AAT CTT UGC ACA ATA CAG CAG |
| | GCT CTT GAA GAG (SEQ ID NO. | AAG CCG C (SEQ ID NO. 80) |
| | 79) | |
| L64C/I68C | ACA CCT CCU TCC TCC CCG AGG | AGG AGG TGU CGC AGG AGG |
| | TCG ACC TCT (SEQ ID NO. 81) | CAG GGC AGG AGG AGA GGA |
| | | AGT CGC G (SEQ ID NO. 82) |
| T146C/M148C | ACC CTC UCC TTC TTC CTC CAC | AGA GGG UGC AGG CGC AGG |
| | CTC GAG AAG C (SEQ ID NO. 83) | AGG GGA AGA AGA TGA AAG |
| | | (SEQ ID NO. 84) |
| E224C | AGG GTT TGU GCG GGG GAC CGA | ACA AAC CCU CGA AGC TAT TTA |
| | TCA GGG AGC TTT (SEQ ID NO. | CGA TAA CA (SEQ ID NO. 86) |
| | 85) | |
| E235C/K238C | ATG CCC GCG GGU TTA CCC GGT | ACC CGC GGG CAU CCC GGG |
| | CGG ACC GCT GAT T (SEQ ID NO. | CAG GGG TGC AAA AGC TCC |
| | 87) | CTG AT (SEQ ID NO. 88) |
| L267C | AG TGG UGC GAC CAG CAG CCA | ACC ACT UCA AGC ACT CAG GCC |
| | CGT GGA T (SEQ ID NO. 89) | GGG C (SEQ ID NO. 90) |
| S363C | AGC CAT GAG UGC ACG GGC GGG | ACT CAT GGC UTA AGA CAT CGA |
| | TTC TTG ACG CAT T (SEQ ID NO. | TCT GG GGG (SEQ ID NO. 92) |
| | 91) | |
| N279C | TT CGT GUG CTT CGG GAG TGG | ACA CGA AUA GGA CGG ATC |
| | TGG GGT C (SEQ ID NO. 93) | CAC GTG GC (SEQ ID NO. 94) |
| V308C | ATG GTG CGU TAG GCC TCC AAA | ACG CAC CAU AGG AAC CTC TGC |
| | CGA CGG CAT TG (SEQ ID NO. 95) | TGG CTG (SEQ ID NO. 96) |
| P390C/Q395C | ATG CAG AGU GCA AGA TGA ACG | ACT CTG CAU AGA GGC ACC ATG |
| | CTG TTA TGC TAA CC (SEQ ID NO. | TAA TTA GTG GTA CCC (SEQ ID |
| | 97) | NO. 98) |
| G460C/T463C | ATT GCT CAU CTT GCC GAG CTC | ATG AGC AAU CTT TGC TCA ATA |
| | TTG AAG AGG TTG CAA A (SEQ ID | CAG CAG AA (SEQ ID NO. 100) |
| | NO. 99) | |
| P48C | ACC TTC GCC GUA TGC ACC AGC | ACG GCG AAG GUG AAG GTG |
| | GGC CCG CCC TCA (SEQ ID NO. | AAG CGC GGA AG (SEQ ID NO. |
| | 101) | 102) |
| S98C | ATG GTT GUC CGC TGC CTC CCC | CA ACC AUG AGG GAC ATG AGA |
| | TCG CTC CGC GAC CTC AT (SEQ | GTC TCG ATT TG (SEQ ID NO. |
| | ID NO. 103) | 104) |
| P13C | ACC GCT CCA UGC CCG CAC GTC | ATG GAG CGG UGG TTG GTG |
| | ATA ATC GTG (SEQ ID NO. 105) | GAG CGG CGG G (SEQ ID NO. |
| | | 106) |
| A66C | ATC GAC ACC UCC TTC CTC CCC | AGG TGT CGA UGG AGC AAG |
| | GAG GTC GAC C (SEQ ID NO. 107) | GGA GGG AGG AGA GGA A (SEQ |
| | | ID NO. 108) |
| T10C | ACC TGC GCU CCA CCA CCG CAC | AGC GCA GGU TGG TGG AGC |
| | GTC ATA (SEQ ID NO. 109) | GGC GGG GGA (SEQ ID NO. |
| | | 110) |
| S112C | ACT CCG CCU GCG GCC GCC GCG | AGG CGG AGU AGG AGG CAA |
| | TCG CCG CC (SEQ ID NO. 111) | TGA GGT CGC (SEQ ID NO. 112) |
| A22P | ATC GTG CCC UCC CCG GGC ATG | AGG GCA CGA UTA TGA CGT |
| | GGC CAC CTC ATC (SEQ ID NO. | GCG GTG GTG G (SEQ ID NO. |
| | 113) | 114) |
| M91I | ATC TCC CUC ATG GTT GTC CGC | AGG GAG AUG AGA GTC TCG |
| | TCC CTC CCC (SEQ ID NO. 115) | ATT TGG GCG (SEQ ID NO. 116) |
| E75P | ACC TCC TUC CTC CCC CCG GTC | AAG GAG GUG TCG ATG GAG |
| | GAC CTC TCC GAC GCC CC (SEQ | GCA GGG AG (SEQ ID NO. 118) |
| | ID NO. 117) | |
| E157P | ACC TCC CGA AGC UTG ATG AAA | AGC TTC GGG AGG UGG AGG |
| | CGG TGT CAT GTG AGT T (SEQ ID | AAG AAG GAG AGG GTC AT (SEQ |
| | NO. 119) | ID NO. 120) |
| G222D | AGG ATT UGG AGG GGG GAC CGA | AAA TCC UCG AAG CTA TTT ACG |
| | TCA GGG (SEQ ID NO. 121) | ATA ACA (SEQ ID NO. 1222) |
| G405D | AGG ACC UGA GGG TGG GAC TCA | AGG TCC UCG GTT AGC ATA ACA |
| | GAC CCT CAG T (SEQ ID NO. 123) | GCG TTC A (SEQ ID NO. 224) |
| G409A | AGG GTG GCA CUC AGA CCC TCA | AGT GCC ACC CUC AGG CCC |
| | GTG GGT AAG GAT GG (SEQ ID | TCG GTT AGC AT (SEQ ID NO. |
| | NO. 125) | 126) |
| G430K | ATA AAA GAG UTG ATG GAA GGT | ACT CTT TTA UAA CTC GTG CGA |
| | GAG GAA GGG AAA C (SEQ ID NO. | TCT CAG C (SEQ ID NO. 128) |
| | 127) | |
| G222D(45) | AGG ATT UGG AGG GGG GAC CGA | AAA TCC UCG AAG CTA TTT ACG |
| | TCA G (SEQ ID NO. 129) | ATA ACA (SEQ ID NO. 130) |
| G405D/G409A/ | AGG ATG GAA UCA TCC GAG GTG | ATT CCA TCC UTA CCC ACT GAG |
| G430K | CTG AGA TCG CAC GAG TTA TAA | GGT CTG AGT GCC ACC CTC |
| | AAG AGT TGA TGG AAG GTG AGG | AGG TCC TCG GTT AGC ATA ACA |
| | AAG GG (SEQ ID NO. 131) | GCG (SEQ ID NO. 132) |
| E75P(46) | ACC TCC TUC CTC CCC CCG GTC | AAG GAG GUG TCG ATG GAG |
| | GAC CTC TCC GAC GCC (SEQ ID | GCA GGG AG (SEQ ID NO. 134) |
| | NO. 133) | |
| E157P(46) | ACC TCC CGA AGC UTG ATG AAA | AGC TTC GGG AGG UGG AGG |
| | CGG TGT CAT G (SEQ ID NO. 135) | AAG AAG GAG AGG GTC A (SEQ |
| | | ID NO. 136) |
| G222D(46) | AGG ATT UGG AGG GGG GAC CGA | AAA TCC UCG AAG CTA TTT ACG |
| | TCA G (SEQ ID NO. 137) | ATA AC (SEQ ID NO. 138) |
| S50D | ACG GCC CGC CCU CAT CCT CCC | AGG GCG GGC CGU CGG TGG |
| | AGC GCG ACT T (SEQ ID NO. 139) | GTA CGG CGA AGG T (SEQ ID |
| | | NO. 140) |
| M94T | ACG GTT GUC CGC TCC CTC CCC | ACA ACC GUG AGG GAC ATG |
| | TCG CT (SEQ ID NO. 141) | AGA GTC TCG ATT T (SEQ ID |
| | | NO. 142) |
| Q86K | ACG CCA AAA UCG AGA CTC TCA | ATT TTG GCG UCG GAG GGG |
| | TGT CCC TCA TGG T (SEQ ID NO. | GCG TCG GAG A (SEQ ID NO. |
| | 143) | 144) |
| Q86R | ACG CCC GUA TCG AGA CTC TCA | ACG GGC GUC GGA GGG GGC |
| | TGT CCC TCA TG (SEQ ID NO. | GTC GGA GA (SEQ ID NO. 146) |
| | 145) | |
| A107K | ATT AAA UCC TAC TCC GCC TCC | ATT TAA UGA GGT CGC GGA |
| | GGC CG (SEQ ID NO. 147) | GCG AGG G (SEQ ID NO. 148) |
| K210R | ATC GTT UGG CCG AGG GTG TTA | AAA CGA UAC CTC TTG GAG TGG |
| | TCG TAA ATA G (SEQ ID NO. 149) | TGG A (SEQ ID NO. 150) |
| Q269E | AGC CAC GUG GAT CCG TCC TAT | ACG TGG CUG CTC GTC CAA CCA |
| | TCG TGA ATT TC (SEQ ID NO. | CTT CAA GCA (SEQ ID NO. 152) |
| | 151) | |
| V285T | ACC CTC AGU ACG GAG CAG CAG | ACT GAG GGU CCC ACC ACT CCC |
| | AAC GAG CTT (SEQ ID NO. 153) | GAA ATT (SEQ ID NO. 154) |
| A299E | AGG TGT GCU GGA ACA CAG CCA | AGC ACA CCU GCA AGC TCG TTC |
| | GCA GAG GTT C (SEQ ID NO. 155) | TGC TGC (SEQ ID NO. 156) |
| Q341R | AGG GGT TCU TGG AGC GTA CCG | AGA ACC CCU CGG GCA GGA |
| | CGG GCA GGG GTT TGG (SEQ ID | GTT TCA ACG (SEQ ID NO. 158) |
| | NO. 157) | |
| K332D/E336K/ | AGG GTT CUT GGA GCG TAC CAA | AGA ACC CUT TGG GCA GGA |
| Q341R/A343K | AGG CAG GGG TTT GGT CTT GCC | GAT CCA ACG GGT CGA TCT CCC |
| | AAT G (SEQ ID NO. 159) | C (SEQ ID NO. 160) |
| S413K | ACC CAA AGU GGG TAA GGA TGG | ACT TTG GGU CTG AGT CCC ACC |
| | AAT CAT CCG AGG (SEQ ID NO. | CTC AGG (SEQ ID NO. 162) |
| | 161) | |
| I472K | AAA AAA UGG GAA AGC AAG GTT | ATT TTT UTG CAA CCT CTT CAA |
| | TAA GGA TCC T (SEQ ID NO. 163) | GAG C (SEQ ID NO. 164) |
| A81L | ACC TGC CCU CCG ACG CCC AAA | AGG GCA GGU CGG AGA GGT |
| | TCG AGA CTC T (SEQ ID NO. 165) | CGA CCT CGG (SEQ ID NO. 166) |
| S110V | CG TGG CCU CCG GCC GCC GCG | AGG CCA CGU AGG AGG CAA |
| | TCG CCG (SEQ ID NO. 167) | TGA GGT CGC (SEQ ID NO. 168) |
| I129F | ATT CGA CGU CGC CCT TGA GCT | ACG TCG AAU GCA TCA GTG |
| | CGG CAT C (SEQ ID NO. 169) | GCG AAA AGG (SEQ ID NO. 170) |
| I188L | ATT TGC UGG ACC CGG TTC AGG | AGC AAA UCC TTG CCG TGG ACC |
| | ATA GGA AGA AC (SEQ ID NO. | GGA AT (SEQ ID NO. 172) |
| | 171) | |
| L333F | AAT TCC UGC CCG AGG GGT TCT | AGG AAT UTC AAC GGG TCG ATC |
| | TGG AGC (SEQ ID NO. 173) | TCC C (SEQ ID NO. 174) |
| M351S | AGG GGT TUG GTC TTG CCA AGC | AAA CCC CUG CCC GCG GTC |
| | TGG GCC CCG CAG ATC GAT GT | TGC TCC AA (SEQ ID NO. 176) |
| | (SEQ ID NO. 175) | |
| F381V | AGC GTG GUC CAT GGG GTA CCA | ACC ACG CUC TCC AGT GTT GAA |
| | CTA ATT ACA TGG (SEQ ID NO. | TTC CAC (SEQ ID NO. 178) |
| | 177) | |
| T388A | ATT GCA UGG CCC CTC TAT GCA | ATG CAA UTA GTG GTA CCC CAT |
| | GAG CAA AAG (SEQ ID NO. 179) | GGA A (SEQ ID NO. 180) |
| S55A | AGC GCG ACU TCC TCT CCT CCC | AGT CGC GCU GGG AGG CTG |
| | TCC CTG CCT (SEQ ID NO. 181) | AGG GCG GGC CGC TGG T (SEQ |
| | | ID NO. 182) |
| A125G | ACC TTT UCG GCA CTG ATG CAA | AAA AGG UCG ACG ACG AGG |
| | TCG ACG TCG CC (SEQ ID NO. | GCG GCG A (SEQ ID NO. 184) |
| | 183) | |
| F140Y | ACA TCT UCT TCC CCT CCA CCG | AAG ATG UAA GGG CGG ATG |
| | CCA T (SEQ ID NO. 185) | CCG AGC TC (SEQ ID NO. 186) |
| GV296/297LG | ACT GGG UCT GGC CCA CAG CCA | ACC CAG UGC AAG CTC GTT CTG |
| | GCA GAG GTT (SEQ ID NO. 187) | CTG C (SEQ ID NO. 188) |
| H300M | AGG TGT GCU GGC CAT GAG CCA | AGC ACA CCU GCA AGC TCG TTC |
| | GCA GAG GTT CCT ATG GG (SEQ | TGC TGC (SEQ ID NO. 190) |
| | ID NO. 189) | |
| E340G | AGG GGT TCU TGG GCC AGA CCG | AGA ACC CCU CGG GCA GGA |
| | CGG GCA GGG GTT T (SEQ ID NO. | GTT TCA ACG (SEQ ID NO. 192) |
| | 191) | |
| F381V-Mut88 | AGC GTG GUC CAT GGG GTA CCA | ACC ACG CUC TCC AGT GTT GAA |
| | CTA ATT GCA TGG (SEQ ID NO. | TTC CAC (= F381V-Reverse; SEQ |
| | 193) | ID NO. 178) |
| A388C/A399C- | AGC AAA AGA UGA ACT GCG TTA | ATC TTT TGC UCT GCA TAG AGG |
| Mut90 | TGC TAA CCG AGG GCC TGA GG | GGC CAG CAA ATT AGT GGT ACC |
| | (= T388C/A399C Forward; SEQ | CCA TGG (SEQ ID NO. 194) |
| | ID NO. 73) | |

### Construction of Plasmid Comprising Mut87

The plasmid of mutant 87 was prepared as a further development of plasmids of earlier mutants:
The plasmid of mutant 87 was prepared using the primers “GV296/297LG” (Table 1) on the plasmid of mutant 86.
The plasmid of mutant 86 was prepared using the primers “G222D” (Table 1) on the plasmid of mutant 85.
The plasmid of mutant 85 was prepared using the primers “E75P” (Table 1) on the plasmid of mutant 81.
The plasmid of mutant 81 was prepared using the primers “G430K” (Table 1) on the plasmid of mutant 80.
The plasmid of mutant 80 was prepared using the primers “T388A” (Table 1) on the plasmid of mutant 77.
The plasmid of mutant 77 was prepared using the primers “S413K” (Table 1) on the plasmid of mutant 74.
The plasmid of mutant 74 was prepared using the primers “I188L” (Table 1) on the plasmid of mutant 71.
The plasmid of mutant 71 was prepared using the primers “S110V” (Table 1) on the plasmid of mutant 48.
The plasmid of mutant 48 was prepared using the primers “Q86K” (Table 1) on the original expression vector pTMH307.

### Construction of Plasmid Comprising Mut88

The plasmid of mutant 88 was prepared using the primers “F381V-Mut88” (Table 1) on the plasmid of mutant 87. See above for details of how the plasmid of mutant 87 was prepared.

### Construction of Plasmid Comprising Mut90

The plasmid of mutant 90 was generated using the primers “A388C/A399C-Mut90” (Table 1) on the plasmid of mutant 88. See above for details of how the plasmid of mutant 88 was prepared.

### Expression and Purification of PtUGT1 WT and Variants

For the expression of PtUGT1 variants 10 ml pre-cultures cells carrying the corresponding expression vector were grown overnight in 2xYT media containing ampicillin (100 μg/ml) and used to inoculate 1 L cultures of 2xYT media with ampicillin selection. Cultures were grown at 37° C. in an MaxQ8000 incubator (Thermo Fisher

Scientific, Germany) at 200 rpm and induced with 0.2 mM isopropyl-β-D-thiogalactopyranoside (IPTG) at OD600 ˜1. Cultures were then grown at 18° C. for 21h for protein expression, and the cells were harvested by centrifugation. The cell pellets were resuspended in 50 mM HEPES pH 7.0, 300 mM NaCl, and 40 mM imidazole pH 8.0.The cell suspension was lysed with 2 cycles through an Avestin Emulsiflex C5 (ATA Scientific Pty Ltd., Australia) homogenizer and treated with DNAse I (Merck). Cells debris was removed by centrifugation at 15000 g for 20 min at 4° C. The cleared extracts were loaded onto Ni Sepharose Fast Flow columns (HisTrap affinity columns, GE Healthcare, U.S.) and the protein was purified using an Äkta FPLC system (GE Healthcare, U.S.). After washing the columns with 20 volumes of buffer (50 mM HEPES pH 7.0, 300 mM NaCl, and 40 mM imidazole pH 8.0), elution was carried out with a 40-500 mM imidazole gradient on the same buffer. The peak fractions were analyzed by SDS-PAGE using NuPAGE™ 4-12% Bis-Tris Protein Gels (Thermo Fisher Scientific, U.S.) stained with Instant Blue (Expedeon Ltd. U.K.), pooled, concentrated using a 50,000 MWCO Amicon Ultra-15 Centrifugal Filter Unit (Merck Millipore, Germany) and stored in 25 mM HEPES pH 7, 50 mM NaCl, and 1 mM DTT.

Final protein concentrations were determined by absorbance measurements at 280 nm using a ND-1000 spectrophotometer (Fischer scientific) and the corresponding theoretical extinction coefficient.

### Tm Experiments—Differential Scanning Fluorometry (DSF)

Melting temperatures (Tm) of PtUGT1 and the variants were measured by DSF using the Protein Thermal Shift Dye Kit (ThermoFisher Scientific) and a qPCR QuantStudio5 machine. Dye solution (1000×) was diluted to final (2×) in Buffer 2× (100 mM HEPES pH7, 100 mM NaCl). 10 μL of dye solution 2× was mixed with 10 μL of protein samples at 0,8 mg/mL in H2O and pipetted in the qPCR multiwell plate. Multiwell plate was centrifuged 30 seconds at 1000 rpm and transferred to the qPCR machine. The protocol initiate with 2 minutes incubation at 25° C., followed by a temperature increase of 0.05° C./second up to 99° C., and a final incubation of 2 minutes at 99° C. The thermal shift assay is a technique that quantifies change in protein denaturation temperature, and is thus used herein to identify mutations beneficial for protein thermal stability. Measurements were carried out in triplicate/quadruplet. Raw data was analyzed with Protein Thermal Shift™ Software v1.x.

### Example 1

### Disulfide Bridge Mutants

### 1.1 Mutant Design

First we focus on introduction of disulfide bridges. In order to do this we use the program SSBOND (Hazes & Dijkstra, 1988) that analyzes protein structures and identifies pairs of residues that could form disulfide bridges if they were mutated to cysteines, based on the distance of Cβ atoms, Cβ/Sγ angles, and Sγ1/Sγ2 angle, and the web-based tool Disulfide by Design 2.0 (Douglas B Craig & Alan A Dombkowski). The programs identified 35 pair of residues having the potential to form intramolecular disulfide bridges and 2 pair of residues having the potential to form intermolecular disulfide bridges. See Table 2 for details of these mutants.

TABLE 2
Disulfide bridge mutants

| Mutant N° | AA mutated |
|---|---|
| 1 | P14C, A117C |
| 2 | S21C, D122C |
| 3 | V76C, R97C |
| 4 | L119C, A132C |
| 5 | V121C, A125C |
| 6 | T126C, R208C |
| 7 | A132C, I137C |
| 8 | A132C, P139C |
| 9 | A147C, P227C |
| 10 | P173C, P181C |
| 11 | I176C |
| 12 | D186C, K396C |
| 13 | P190C, A198C |
| 14 | D193C, N196C |
| 15 | A259C, L264C |
| 16 | P271C, S274C |
| 17 | G273C, G365C |
| 18 | V278C, W307C |
| 19 | L286C, Q290C |
| 20 | G337C, E340C |
| 21 | T342C, G346C |
| 22 | L368C, I387C |
| 23 | T388C, A399C |
| 24 | P412C, E424C |
| 25 | V455C, S462C |
| 26 | S457C, G460C |
| 27 | L64C, I68C |
| 28 | T146C, M148C |
| 29 | A147C, E224C |
| 30 | E235C, K238C |
| 31 | L267C, S363C |
| 32 | N279C, V308C |
| 33 | P390C, Q395C |
| 34 | G460C, T463C |
| 35 | P48C, S98C |
| 36 | A66C, P13C |
| 37 | S112C, T10C |
| 13 + 23 | P190C, A198C, T388C, A399C |
| 13 + 28 | P190C, A198C, T146C, M148C |
| 23 + 28 | T388C, A399C, T146C, M148C |
| 13 + 23 + 28 | P190C, A198C, T388C, A399C, T146C, M148C |

### 1.2 Melting Temperature

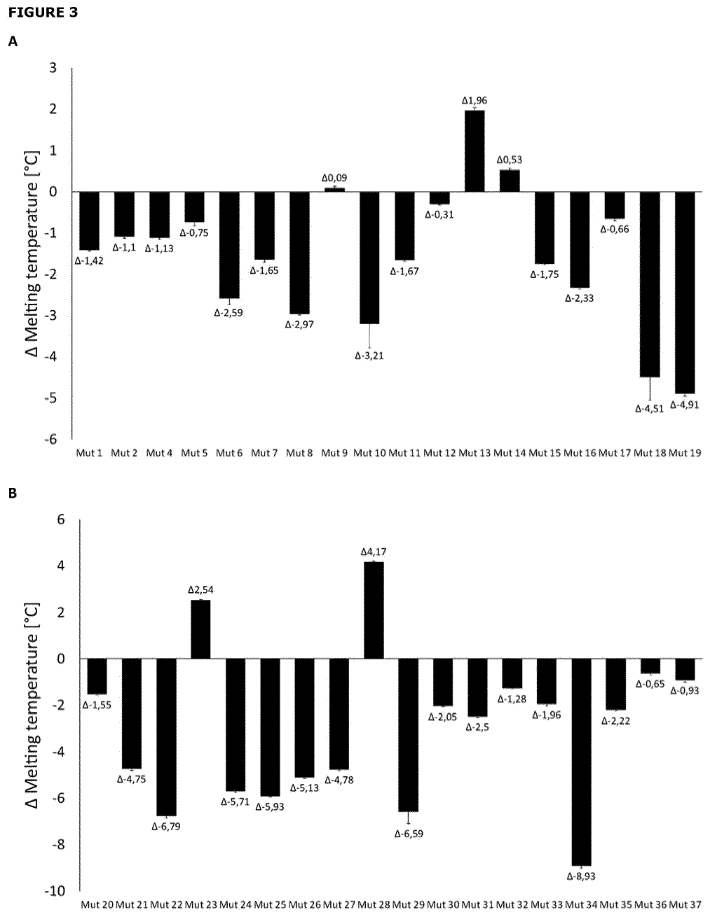

Melting temperatures (Tm) of PtUGT1 wild type and the disulfide bridge mutants were measured as described above. The results are illustrated in FIG. 3, where it can be seen that though most mutants performed about the same (or worse) than the wildtype, there were also a few mutants which had increased Tm compared to the wild type—such as mut13, mut23, and mut28.

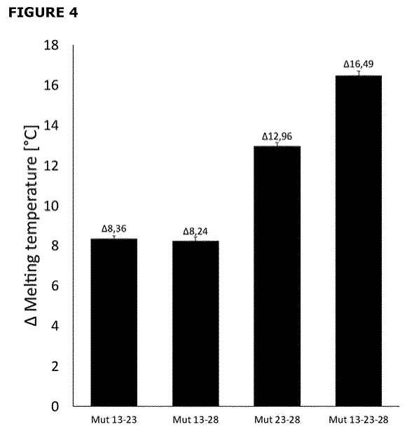

These best performing mutations were combined as different double mutants or a triple mutant. These mutants had higher Tm than any of single mutants (see FIG. 4), but unfortunately performed poorly in terms of activity (data not shown).

### Example 2

### Consensus Mutants

### 2.1 Mutant Design

Secondarily a consensus approach was used. A multiple sequence homology alignment was performed by first collecting sequences of PtUGT1 homologues of 60% or higher sequence identity, using NCBI sequence blast search. Subsequently, a multiple sequence alignment of all the sequences was created using Multiblast ClustalW2 (ref?), the alignment columns were manually/visually scanned and the positions where the original PtUGT1 amino acid was under-represented identified. Leveraging the structure of PtUGT1 (5NLM) and of two of the homologs (2ACV and 2VG8) used for the consensus approach, a rational analysis of the potential mutations was performed and the final number of variants was set on 34. Mutants of PtUGT1 were constructed to make a specific position more alike the majority of known homologues. See Table 3 for details of these mutants.

TABLE 3
Consensus mutagenesis mutants

| Mutant No | AA mutated | Comments |
|---|---|---|
| PtUGT1 WT | Wild type enzyme (no mutations) | |
| 38 | A22P, M91I | Increase |
| 39 | E75P | Pro/Gly |
| 40 | E157P | ratio |
| 41 | G222D | |
| 42 | G405D | |
| 43 | G409A | |
| 44 | G430K | |
| 45* | G222D, G405D, G409A, G430K | |
| 46** | E75P, E157P, G222D, G405D, G409A, | |
| | G430K | |
| 47 | S50D, M94T | Extra polar |
| 48 | Q86K | interactions |
| 48B | Q86R | |
| 49 | A107K | |
| 50 | K210R | |
| 51 | Q269E | |
| 52 | V285T | |
| 53 | A299E, Q341R | |
| 54 | K332D, E336K, Q341R, A343K | |
| 55 | S413K | |
| 56 | S472K | |
| 57 | A81L | Improve |
| 58 | S110V | hydrophobic |
| 59 | I129F | packing |
| 60 | I188L | |
| 61 | L333F | |
| 62 | M351S | |
| 63 | F381V | |
| 64 | T388A | |
| 65 | S55A | Unclasified |
| 66 | A125G | |
| 67 | F140Y, 1472K | |
| 68 | GV296/297LG | |
| 69 | H300M | |
| 70 | E340G | |

*Mut45 = Mut(41 + 42 + 43 + 44)
**Mut46 = Mut(39 + 40 + 41 + 42 + 43 + 44)

### 2.2 Melting Temperature

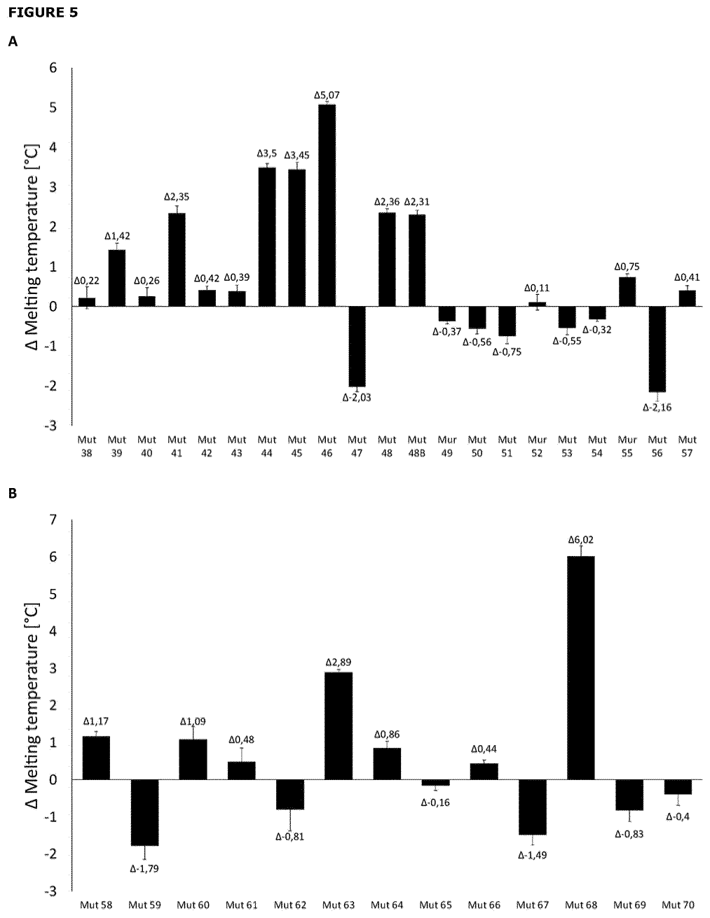

Melting temperatures (Tm) of PtUGT1 wild type and the consensus mutants were measured as described above. The results are illustrated in FIG. 5. For the consensus mutagenesis enzymes, more mutants were found which had increased Tm compared to the wild type-such as mut39, mut41, mut44, mut45, mut46, mut48, mut48B, mut55, mut58, mut60, mut63, mut64, and mut68.

### Example 3

### Combination of Mutations

### 3.1 Mutant Design

Different combinations of mutations were tested, as specified in Table 4.

TABLE 4
Combination of mutants

| Mutant No | AA mutated |
|---|---|
| PtUGT1 WT | Wild type enzyme (no mutations) |
| 71 | Q86K, S110V |
| 72 | S413K, T388A |
| 73 | F381V, GV296/297LG |
| 74 | Q86K, S110V, I188L, T388A |
| 75 | S413K, T388A, I188L |
| 76 | F381V, GV296/297LG, I188L |
| 77 | Q86K, S110V, I188L, S413K |
| 78 | Q86K, S110V, I188L |
| 79 | F381V, GV296/297LG, I188L, G430K |
| 80 | Q86K, S110V, I188L, S413K, T388A |
| 81 | Q86K, S110V, I188L, S413K, T388A, G430K |
| 82 | E75P, G430K |
| 83 | E75P, G222D |
| 84 | E75P, G222D, G430K |
| 85 | Q86K, S110V, I188L, S413K, T388A, G430K, E75P |
| 86 | Q86K, S110V, I188L, S413K, T388A, G430K, E75P, |
| | G222D |
| 87 | Q86K, S110V, I188L, S413K, T388A, G430K, E75P, |
| | G222D, GV296/297LG |
| 88 | Q86K, S110V, I188L, S413K, T388A, G430K, E75P, |
| | G222D, GV296/297LG, F381V |
| 89 | Q86K, S110V, I188L, S413K, T388A, G430K, E75P, |
| | G222D, GV296/297LG, F381V, T146C, M148C |
| 90 | Q86K, S110V, I188L, S413K, G430K, E75P, G222D, |
| | GV296/297LG, F381V, T388C, A399C |
| 91 | E75P, G430K, Q86K |
| 92 | E75P, G430K, Q86K, GV296/297LG |
| 93 | E75P, G430K, Q86K, T388C, A399C |
| 94 | E75P, G430K, Q86K, T146C, M148C |
| 96 | Q86K, S110V, I188L, S413K, G430K, E75P, G222D, |
| | T388C, A399C |
| 97 | Q86K, S110V, I188L, S413K, T388A, G430K, E75P, |
| | G222D, T146C, M148C |
| 98 | Q86K, S110V, I188L, S413K, G430K, E75P, G222D, |
| | GV296/297LG, T388C, A399C |
| 99 | Q86K, S110V, I188L, S413K, T388A, G430K, E75P, |
| | G222D, GV296/297LG, T146C, M148C |

### 3.2 Melting Temperature

Melting temperatures (Tm) of PtUGT1 wild type and the combination mutants were measured as described above. The results are illustrated in FIG. 6 and further summarized in table 4. For the combination mutant enzymes, mutants were found which had further increased Tm compared to the wild type—as high as 15° C. increase in melting temperature was obtained for mut90, compared to wild type.

The best performing mutants were selected for further studies: mut87, mut88, mut90. The selection was primarily based on increase in melting temperature as well as relative activity measured compared to wild type.

All specified mutations are with reference to wild type PtUGT (SEQ ID NO 2). Black shade in the table means that the mutation is present in the mutant.

ΔTm is change in Tm compared to wild type enzyme (in 50 mM HEPES, 50 mM NaCl at pH 7). Relative activity is mutant activity compared to wildtype activity at 40° C. (in Citrate-Phosphate buffer at pH 7)

3.3 Relative Activity at Different temperatures

Relative activity experiments were also carried out at 40, 55 and 60° C.

Calculation of relative activity of PtUGT1 variants (mut87, mut88, and mut90) compared with WT activity were performed in reactions (triplicate) with end point measurements of product formation using the model substrate 3,4-Dichlorophenol (DCP). Product peak was monitored via reverse phase HPLC, using an Ultimate 3000 Series apparatus (Thermo Scientific) and a kinetex 2.6 μm C18 100 Å 100×4.6 mm analytical column (Phenomenex). MilliQ water and acetonitrile containing 0.1% formic acid were used as mobile phases A and B, respectively. PCR strip tubes containing 200 μl of reaction mixture (1 mM UDP-glucose, 50 mM citrate-phosphate buffer pH 7, 500 μM DCP and 1 μg enzyme) were incubated at 40° C. for 10 minutes. Reactions were stopped and analyzed at 290 nm using a multi-step program (starting at 5% B, ramp up to 25% B at 1.5 min, ramp up to 30% B at 3.5 min, ramp to up 100% B at 6.25 min, stay at 100% B until 7 min, gradient decrease to 0% B at 8 min, stay at 0% B until 9 min). Peak integration and data handling was performed using the Chromeleon software (Thermo Scientific).

The three mutants have comparable or slightly reduced activity to the wildtype enzyme at 40° C. (FIG. 7A). However, at 55° C. and 60° C. the mutants significantly outperform the wild type enzyme, which has no activity at these temperatures (FIGS. 7B and 7C).

At 55° C. the mutants perform better or at least comparable to the wildtype activity measured at 40° C. At 60° C. the mutants still maintain half the activity as recorded for the wildtype at 40° C.

### 3.4 Half-Life

Half/shelf-life times are defined as the amount of time that an enzyme can be pre-incubated at a defined temperature having as a results a 50% residual activity compared with the activity without the pre-incubation.

For determination of the half/shelf-life of PtUGT1 WT and variants, stocks of free enzyme in buffer (100 mM HEPES pH7, 100 mM NaCl) were incubated either at 45° C. or room temperature for different period of times, and their residual activities were analyzed using the same procedure described for the “relative activity experiment” and compared against the activity without the pre-incubation step.

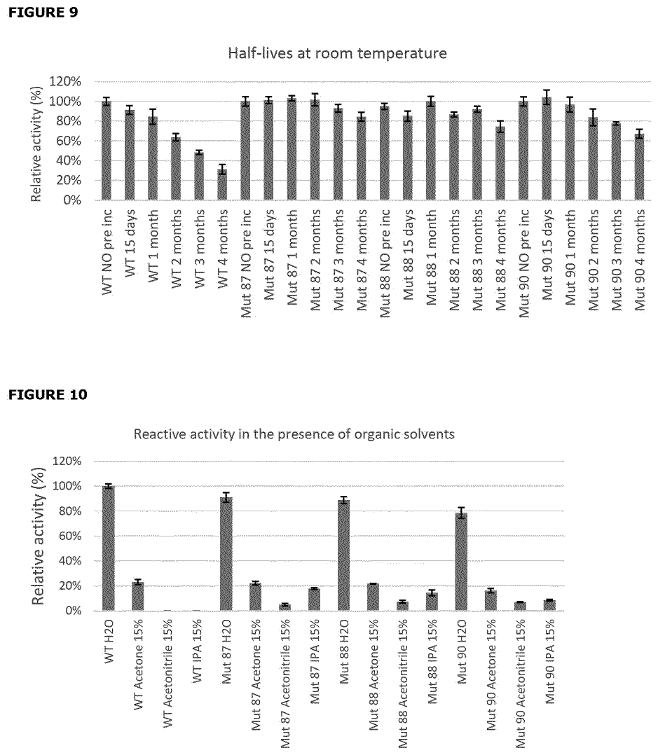

At 45° C., the residual activity of the wild-type enzyme is reduced by >90% after 5 h 20 min. Meanwhile, at this same incubation period, the residual activity of the mutant enzymes remain rather constant. A drop to approx. 60% is seem after 24 hours, and after 96 h the residual activity is down to approx. between 35-45%. See FIGS. 8 and 9

### 3.5 Solvent Tolerance

For determination of the solvent tolerance of PtUGT1 WT and variants, relative activities were analyzed using the same procedure described for the “relative activity experiment” with the addition of either 15% v/v acetone, acetonitrile or isopropanol, and compared against the activity without addition of any organic solvent. As seen in FIG. 10, the mutants pertain some activity in all three solvents, while the wildtype shows no activity in acetonitrile and isopropanol.

### 3.6 Chemo-Stability

Chemo-stability is defined as the property of a polypeptide to retain structural integrity and activity in presence of chemicals such as indoxyl, indoxyl derivatives or DCP. Chemo-stability is here tested against DCP, which is usually considered “harsh” for the enzyme, and may reduce or destroy the activity of the enzyme.

Reactions were performed in presence of 15 mg/L enzyme (PtUGT1 WT or Mutant 87), 4 mM 3,4-dichlorophenol (DCP), 6 mM UDP-Glc and 0.5 M citrate pH 6.2. 50 μL reactions were set up in HPLC vials with insert and incubated at 20 degrees for 48 h prior to analysis via reverse phase HPLC, using an Ultimate 3000 Series apparatus (Thermo Scientific) and a kinetex 2.6 μm C18 100 Å 100×4.6 mm analytical column (Phenomenex). MilliQ water and acetonitrile containing 0.1% formic acid were used as mobile phases A and B, respectively. Monitoring and data handling was operated using the Chromeleon software (Thermo Scientific). The method used for the separation of analytes had a flow rate of 1 mL/min and started at 2% B for 30 seconds, followed for 1 minute of 35% B and then a gradient from 35% to 80% B for 1.5 min. After, B was increased to 98% for 1.2 min and finally reduced to 2% B for the last 0.8 minute. DCP and its glucoside were detected at 280 nm.

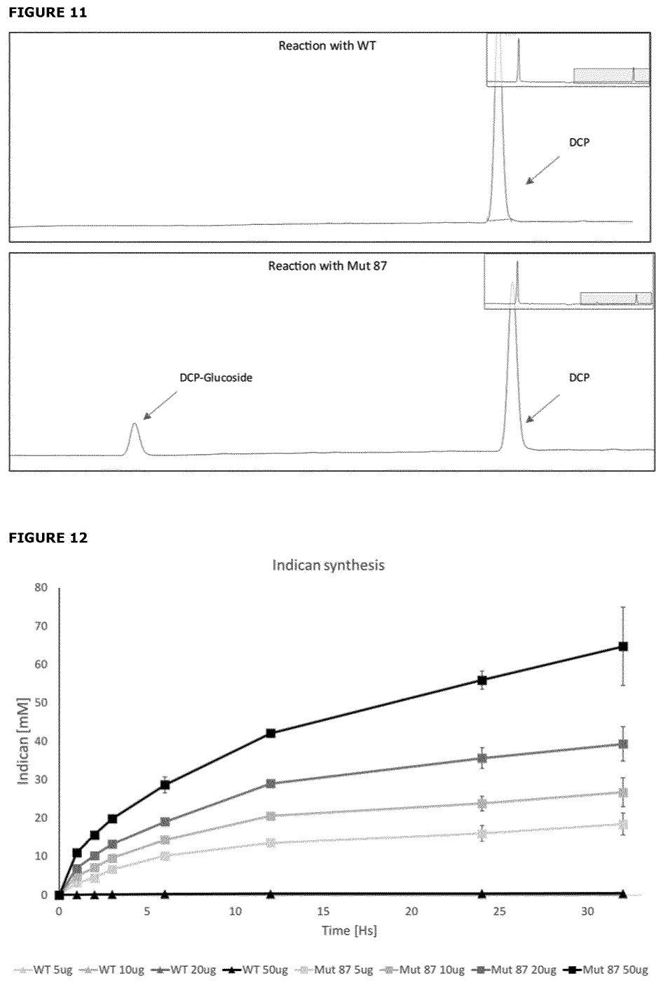

In FIG. 11, the HPLC chromatogram shows that in the reaction done with the WT enzyme, there is only substrate present, no product at all. So the WT enzyme is not able to withstand the higher concentration of DCP and therefore it is inactive. Similar lack of chemostability is reported by Petermeier et al 2021. In the reaction done with Mut87, there is a clear peak of the product DCP-glucoside, which means the Mut87 its chemo-stable against this compounds, whereas the WT is not.

### Example 4

### Demin Dying

4.1 Synthesis of Indican from Indoxyl Acetate

The proof of concept for the synthesis of indican from high concentrations of indoxyl-acetate (100 mM) was performed in triplicate inside an anaerobic chamber, using glass HPLC vials stirred with small magnets and at 30° C. Reaction consisted on 3.5 mg indoxyl-acetate, 90 mM buffer phosphate-citrate pH8, 1 mM UDP, 200 mM sucrose, 2U of Esterase from Bacillus subtilis (Sigma Aldrich), and different concentrations of PtUGT1/SuSy always at a molar ratio of 1:5 (50 μg, 20 μg, 10 μg, 5 μg for PtUGT1 WT or Mut 87; and 432,5 μg, 173 μg, 86,7 μg, 43,3 μg for SuSy). Sucrose synthase (SuSy) converts sucrose and uridine 5′-diphosphate (UDP) into UDP-glucose. The reaction was started by the addition of all three enzymes (Esterase, PtUGT1 and SuSy) and the progression was followed by HPLC using the same method used in “Relative activity experiment”. Samples were collected at 1, 2, 3, 6, 12, 24, and 32 hours.

FIG. 12 shows that WT enzymes does not produce any indican from indoxyl acetate under the conditions tested herein, with Mut87 enzyme does indeed produce indican from indoxyl acetate. It is speculated that the high concentration of substrate inactivates the WT enzyme; while this does not seem to pose a problem for the mutant enzyme.

### 4.2 Demin Dyeing

Discs of 20 square centimeters of ready-to-dye denims (radius 1.784 cm, diameter 3.57 cm, weight 802+/−2 mg) are dyed in 3 ml of water at pH 9 with 30 μmol indican (prepared above) and 1 mg of Rye β-glucosidase 1 (SEQ ID NO. 9). Discs are turned over every 5 min at room temperature for 15 min, and left for 1 h at room temperature before being washed with water and soap and dried overnight at room temperature.

As seen in figure FIG. 13, the produced indican is capable of dyeing denim. The coloration is further specified in CIEL table 6.

TABLE 6
CIEL values for dyed textiles

| Sample | L* | a* | b* |
|---|---|---|---|
| 10 μmol Indican Back | 69.74 | −5.77 | −11.18 |
| 10 μmol Indican Front | 74.84 | −5.27 | −8.26 |
| 20 μmol Indican Back | 64.25 | −5.84 | −11.37 |
| 20 μmol Indican Front | 66.20 | −5.70 | −12.12 |
| 30 μmol Indican Back | 56.57 | −5.22 | −14.09 |
| 30 μmol Indican Front | 55.31 | −7.03 | −15.14 |
| 40 μmol Indican Back | 52.37 | −5.41 | −15.05 |
| 40 μmol Indican Front | 51.98 | −6.30 | −14.86 |
| 60 μmol Indican Back | 53.04 | −6.50 | −14.79 |
| 60 μmol Indican Front | 52.16 | −5.56 | −16.32 |
| 100 μmol Indican Back | 46.48 | −6.54 | −14.35 |
| 100 μmol Indican Front | 48.42 | −4.56 | −18.03 |

### Example 5

### Glycosylation of other Indoxyl Derivatives

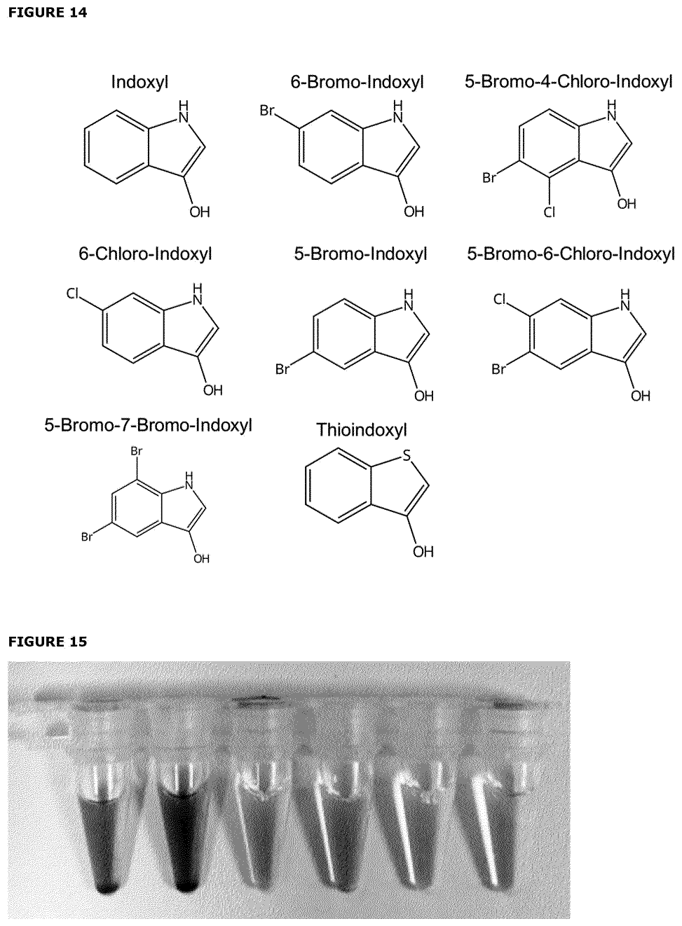

In the example above, it was demonstrated that the UGT mutants of the present invention are capable of glycosylating indoxyl. We herein further demonstrate that the UGT mutants are also able to glycosylating other indoxyl derivatives. See FIG. 14 for graphical illustration of a selection of such indoxyl derivatives of interest.

### 5.1 6-Bromo-Indoxyl

Reactions performed in strip tubes of PCR at 30° C. Reaction components are specified in table 7. Initiated with addition of esterase from Bacillus subtilis in buffer with multichannel pipette.

TABLE 7
Reaction components for glycosylation of 6-bromo-indoxyl

| | | | | Added | |
|---|---|---|---|---|---|
| Component | Stock concentration | End concentration | | volume | |
| 6-Bromo-Indoxyl | 3.5 mM in H20 | 1.75 | mM | 75 | ul |
| acetate** | | | | | |
| UDP-Glucose | 100 mM in H20 | 5 | mM | 7.5 | ul |
| UGT* | | | | 1.5 | ul |
| Esterase from | 0.1 U/ul in buffer 2x | 0.2 U reaction | | 2 | ul |
| Bacillus subtilis | | | | | |

| | | | | |
|---|---|---|---|---|
| Buffer 2x | HEPES 100 | HEPES 44 | 64 | ul |
| | mM pH7; | mM pH7, | | |
| | 100 mM NaCl | NaCl 44 mM | | |
| | | TOTAL VOLUME | 150 | ul |

*UGT stocks: WT = 9.04 mg/ml; Mut 87 = 1.57 mg/ml; Mut 88 = 12.32 mg/ml; Mut 90 = 4.4 mg/ml. Storage buffer: 25 mM HEPES pH7, 50 mM NaCl.
**6-Bromo-Indoxyl-Acetate 3.5 mM in water dissolved in bath sonicator at room temperature.

When 6-Bromo-Indoxyl acetate is treated with esterase enzyme, acetate and 6-Bromo-Indoxyl form. If further exposed to air, a dimer spontaneously forms from 6-bromo-indoxyl, which has a distinct purple color (known as Tyrian purple or Royal purple). As evidenced in FIG. 15, it is clear that this purple color develops for the two negative controls, where either no UGT enzyme or no UDP-glucose is added. Meanwhile, for the samples where UGT enzyme is added, the 6-Bromo-Indoxyl is glycosylated (after acetate removal) and thereby “prevented” from forming the dimer, hence no color formation.

It is thereby shown that PtUGT1 (wild type) and all tested mutants are active on 6-Bromo-Indoxyl.

5.2 5-Bromo-4-chloro-3-Indoxyl

Reactions performed in strip tubes of PCR at 30° C. Reaction components are specified in table 8. Initiated with addition of esterase from Bacillus subtilis in buffer with multichannel pipette.

TABLE 8
Reaction components for glycosylation
of 5-bromo-4-chloro-3-indoxyl

| | | End | | Added | |
|---|---|---|---|---|---|
| Component | Stock concentration | concentration | | volume | |
| 5-bromo-4-chloro-3- | 2.5 mg/ml (8.6 mM) in | 0.057 | mM | 1 | ul |
| indoxyl-Acetate | DMSO | | | | |
| UDP-Glucose | 100 mM in H20 | 5 | mM | 7.5 | ul |
| UGT | see above* | | | 5 | ul |
| Esterase from | 0.1 U/ul in buffer 2x | 0.05 U | | 0.5 | ul |
| Bacillus subtilis | | reaction | | | |
| H2O | | | | 61 | ul |

| | | | | |
|---|---|---|---|---|
| Buffer 2x | HEPES 100 mM PH7; | HEPES 50 mM | 75 | ul |
| | 100 mM NaCl | PH7, NaCl | | |
| | | 50 mM | | |
| | | TOTAL | 150 | ul |
| | | VOLUME | | |

*UGT stocks: WT = 9.04 mg/ml; Mut 87 = 1.57 mg/ml; Mut 88 = 12.32 mg/ml; Mut 90 = 4.4 mg/ml. Storage buffer: 25 mM HEPES pH7, 50 mM NaCl.
**5-bromo-4-chloro-indoxyl-acetate 3.5 mM in water dissolved in bath sonicator at room temperature.

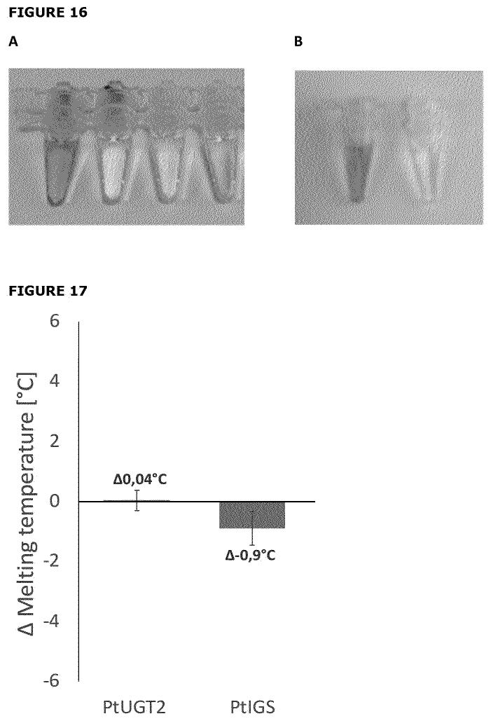

When 5-Bromo-4chloro-Indoxyl acetate is treated with esterase enzyme, acetate and 5-Bromo-4chloro-Indoxyl form. If further exposed to air, a dimer spontaneously forms from 5-Bromo-4chloro-Indoxyl, which has a bright blue color. As evidenced in FIG. 16, it is clear that this blue color develops for the negative control, where no UGT enzyme is added. Meanwhile, for the samples where UGT enzyme is added, the 5-Bromo-4chloro-Indoxyl is glycosylated (after acetate removal) and thereby “prevented” from forming the dimer, hence no color formation.

It is thereby shown that PtUGT1 (wild type) and all tested mutants are active on 5-Bromo-4chloro-Indoxyl.

### 5.3 Further Indoxyl Derivatives

6-chloro-indoxyl, 5-bromo-indoxyl, 5-bromo-6-chloro-indoxyl, thioindoxyl, and 5,7-dibromo-indoxyl are also glycosylated by an enzyme of the present invention. This may be demonstrated in a similar manner as shown above in section 5.1 and 5.2, where the acetate-form of the molecules are used as starting material, and an esterase enzyme is used in combination with the UTG enzyme. The spontaneous dimerization of the indoxyl-derivatives is prevented by the action of the UGT enzyme, resulting in glycosylation of the compounds.

### Example 6

### Melting Temperatures and Chemostability of Prior Art UGT Enzymes

The following prior art UGT enzymes were tested:
PtUGT2 (SEQ ID NO. 200): P. tinctorium UGT isoform 2 (disclosed as sequence #4 in WO2016/141207). PtUGT2 has five mutations compared to SEQ ID NO. 2: delS1, V19M, G225A, E230Q, and A423D.
PtIGS (SEQ ID NO. 202): Persicaria tinctoria glycosyltransferase; Uniprot ref. A0A2L2R220. PtIGS has three mutations compared to SEQ ID NO. 2: delS1, V19M, and A423D.

The enzymes were expressed and purified as disclosed herein.

Melting temperatures (Tm) of prior art UGT enzymes were measured as described herein (section ‘general methodology’). The results are summarized in table 9 and illustrated in FIG. 17, where the Δ(Tm) is with respect to the PtUGT1 WT (SEQ ID NO. 2).

TABLE 9
Melting temparatures

| | Tm (° C.) | ΔTm (° C.) compared to WT PtUGT1 |
|---|---|---|
| WT PtUGT1 | 52.45 ± 0.05 | |
| PtUGT2 | 52.49 ± 0.34 | 0.04 |
| PtIGS | 51.55 ± 0.56 | −0.90 |

It was found that the melting temperature of PtUGT2 did not differ significantly from the WT PtUGT1. while PtIGS had lower melting temperatures than PtUGT1 WT.

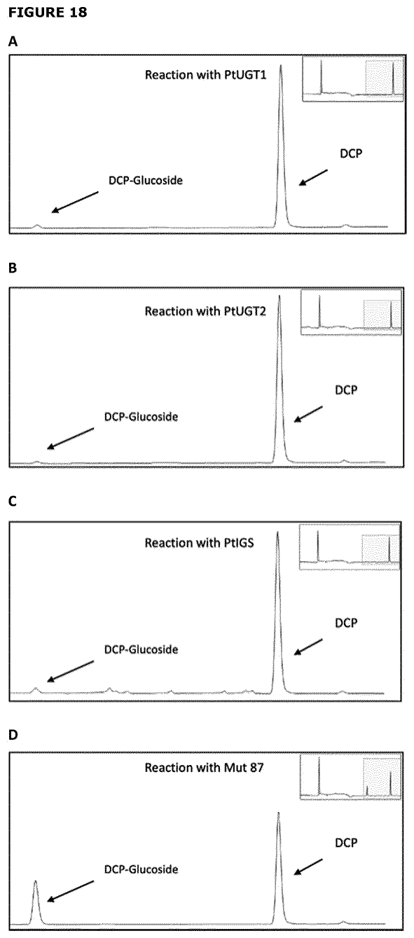

Chemostability was measured as described herein (example 3.6). The results are presented in FIG. 18. The HPLC chromatograms show that in the reaction done with Mut87, there is a clear peak of the product DCP-glucoside, which means Mut87 its chemo-stable against this compounds; whereas in the reactions done with the wild type enzyme and other prior art enzymes, only a very small DCP-glucoside peak is found, hence the prior art enzymes are not able to withstand the high concentration of DCP and are therefore inactive.

### REFERENCES

Lee et al 2004. One-pot enzymatic synthesis of UDP-D-glucose from UMP and Glucose-1-Phosphase using ATP regeneration system. Journal of Biochemistry and Molecular Biology, Vol. 37, No. 4, July 2004, pp. 503-506

Frederik De Bruyn et al. Development of an in vivo glucosylation platform by coupling production to growth: Production of phenolic glucosides by a glycosyltransferase of Vitis vinifera. Biotechnol. Bioeng (2015) 112, 1594-1603, https://doi.org/10.1002/bit.25570
Craig, D. B., Dombkowski, A. A. Disulfide by Design 2.0: a web-based tool for disulfide engineering in proteins. BMC Bioinformatics 14, 346 (2013). doi.org/10.1186/1471-2105-14-346
B W Dijkstra. Model building of disulfide bonds in proteins with known three-dimensional structure B Hazes 1, PMID: 3244694 DOI: 10.1093/protein/2.2.119
Hsu, T. M. et al. Employing a biochemical protecting group for a sustainable indigo dyeing strategy. Nat. Chem. Biol. 14, 256-261 (2018).
Inoue et al 2017. Characterization of UDP-glucosyltransferase from Indigofera tinctoria. Plant Physiol Biochem. 2017 December;121:226-233. doi: 10.1016/j.plaphy.2017.11.002. Epub 2017 Nov. 6.
Philipp Petermeier, Cristina Fortuna, Kathrine M. Hübschmann, Gonzalo N. Bidart, Thomas Tørring, David Teze, Ditte H. Welner, and Selin Kara. ACS Sustainable Chemistry & Engineering 2021 9 (25), 8497-8506. DOI: 10.1021/acssuschemeng.1c01536

1. A polypeptide having glycosyltransferase activity (enzyme classification EC: 2.4.1.-), wherein the amino acid sequence of said polypeptide has at least 75% sequence identity with SEQ ID NO. 2, and wherein said amino acid sequence comprises (i) one or more amino acid residue substitutions selected from: E75P, Q86K, S110V, I188L, G222D, G296L, V297G, F381V, T388A, S413K and G430K with respect to SEQ ID NO. 2, and/or (ii) amino acid residue substitutions T388C and A399C with respect to SEQ ID NO. 2.

2. The polypeptide according to claim 1, wherein the half-life at 45° C. of said glycosyltransferase activity of said polypeptide is increased, compared to SEQ ID NO. 2.

3. The polypeptide according to claim 1, wherein said amino acid sequence comprises amino acid residue substitutions E75P, Q86K, S110V, I188L, G222D, G296L, V297G, S413K, and G430K with respect to SEQ ID NO. 2.

4. The polypeptide according to claim 3, wherein said amino acid sequence further comprises (i) one or more amino acid residue substitutions selected from F381V and T388A with respect to SEQ ID NO 2, and/or (ii) amino acid residue substitutions T388C and A399C with respect to SEQ ID NO. 2.

5. The polypeptide according to claim 3, wherein said amino acid sequence further comprises
(i) amino acid residue substitution T388A with respect to SEQ ID NO. 2, or
(ii) amino acid residue substitutions F381V and T388A with respect to SEQ ID NO. 2, or
(iii) amino acid residue substitutions F381V, T388C, and A399C with respect to SEQ ID NO. 2.

6. A composition comprising (i) a polypeptide having glycosyltransferase enzyme activity according to claim 1, (ii) a compound comprising a reactive group, and (iii) a nucleotide sugar.

7. The composition according to claim 6, wherein the compound is an indoxyl compound; and preferably wherein said composition comprises less than 2% free oxygen.

8. A composition according to claim 6, wherein the nucleotide sugar is an UPD-glucose.

9. A kit of parts comprising (i) a polypeptide having glycosyltransferase enzyme activity according to claim 1, and (ii) a polypeptide having beta-glucosidase enzyme activity (enzyme classification EC 3.2.1.21).

10. A method for glycosylating a compound, comprising the steps of
a. providing (i) a compound comprising a reactive group, (ii) a polypeptide having glycosyltransferase activity according to claim 1, and (iii) a nucleotide sugar,
b. mixing the components (i), (ii), and (iii) provided in step (a) to obtain a mixture, and
c. letting the mixture react to obtain a glycosylated compound.

11. The method according to claim 10, wherein the compound provided in step (a)(i) is an indoxyl compound, wherein the glycosylated compound obtained in step (c) is a soluble glycosylated indoxyl dye-precursor, and wherein steps (b) and (c) are preferably carried out under reaction conditions wherein less than 2% free oxygen is present.

12. The method according to claim 11, wherein the indoxyl compound is selected from the group consisting of indoxyl, 6-bromo-indoxyl, 5-bromo-4-chloro-indoxyl, 6-chloro-indoxyl, 5-bromo-indoxyl, 5-bromo-6-chloro-indoxyl, thioindoxyl, and 5-bromo-7-bromo-indoxyl.

13. The method according to claim 10, wherein the nucleotide sugar is UDP-glucose.

14. A method for dying a product, comprising the steps of
a. providing (i) an indoxyl compound, (ii) a polypeptide having glycosyltransferase enzyme activity according to anyone of claims 1-5, (iii) a nucleotide sugar, and (iv) a polypeptide having beta-glucosidase enzyme activity (enzyme classification EC 3.2.1.21),
b. mixing components (i), (ii), and (iii) provided in step (a) to obtain a mixture, preferably at reaction conditions wherein less than 2% free oxygen is present,
c. letting the mixture react to obtain a soluble glycosylated indoxyl dye-precursor,
d. mixing said dye precursor with said product and said beta-glucosidase under reaction conditions wherein free oxygen is present, to obtain a dyed textile.
wherein said product is selected from the group consisting of yarn, textiles, and fabrics.

15. Use of a polypeptide having glycosyltransferase enzyme activity according to claim 1 for glycosylating a compound, wherein said compound comprises a reactive group.

16. The use according to claim 15, wherein the compound is an indoxyl compound, and wherein the glycosylated compound is for use in a textile dying process.


SEQUENCE LISTING (202 sequences)

SEQ ID NO: 1 - DNA, 1437 bp, Persicaria tinctoria
  source 1..1437: mol_type = genomic DNA
  CDS 1..1437: note = PtUGT1 wild type enzyme from Polygonum tinctorium/Persicaria tinctoria
atgtctgaatcccccgccgctccaccaaccaccgctccaccaccgcacgtcataatcgtgccctccgccggcatgggccacctcatccccctcgccgagttcgccaagcgcctccttccgcgcttcaccttcaccttcgccgtacccaccagcggcccgccctcatcctcccagcgcgacttcctctcctccctccctgcctccatcgacacctccttcctccccgaggtcgacctctccgacgccccctccgacgcccaaatcgagactctcatgtccctcatggttgtccgctccctcccctcgctccgcgacctcattgcctcctactccgcctccggccgccgcgtcgccgccctcgtcgtcgaccttttcgccactgatgcaatcgacgtcgcccttgagctcggcatccgccctttcatcttcttcccctccaccgccatgaccctctccttcttcctccacctcgagaagcttgatgaaacggtgtcatgtgagtttgccgagctgtccgacccggttcagatccccgggtgtattccggtccacggcaaggatttgatcgacccggttcaggataggaagaacgacgcctacaagtggctcctccaccactccaagaggtataaattggccgagggtgttatcgtaaatagcttcgagggtttggaggggggaccgatcagggagcttttgcaccccgagccgggaaagccgcgggtttacccggtcggaccgctgattcaggccggttcgtgcgagaagggggcagctgcccggcctgagtgcttgaagtggttggaccagcagccacgtggatccgtcctattcgtgaatttcgggagtggtggggtcctcagtacggagcagcagaacgagcttgcaggtgtgctggcccacagccagcagaggttcctatgggtggttaggcctccaaacgacggcattgccaacgccacgtatttcagcgtcgacggggagatcgacccgttgaaactcctgcccgaggggttcttggagcagaccgcgggcaggggtttggtcttgccaatgtgggccccgcagatcgatgtcttaagccatgagtcgacgggcgggttcttgacgcattgtgggtggaattcaacactggagagcgtgttccatggggtaccactaattacatggcccctctatgcagagcaaaagatgaacgctgttatgctaaccgagggcctgagggtgggactcagaccctcagtgggtaaggatggaatcatccgaggtgctgagatcgcacgagttataggggagttgatggaaggtgaggaagggaaacgaatacggagtaagatgcaggagttgaagcgtgcggcttctgctgtattgagcaaagatggatcatctactcgagctcttgaagaggttgcaaaaatttgggaaagcaaggtttaa

SEQ ID NO: 2 - protein, 477 aa, Persicaria tinctoria
SESPAAPPTTAPPPHVIIVPSAGMGHLIPLAEFAKRLLPRFTFTFAVPTSGPPSSSQRDFLSSLPASIDTSFLPEVDLSDAPSDAQIETLMSLMVVRSLPSLRDLIASYSASGRRVAALVVDLFATDAIDVALELGIRPFIFFPSTAMTLSFFLHLEKLDETVSCEFAELSDPVQIPGCIPVHGKDLIDPVQDRKNDAYKWLLHHSKRYKLAEGVIVNSFEGLEGGPIRELLHPEPGKPRVYPVGPLIQAGSCEKGAAARPECLKWLDQQPRGSVLFVNFGSGGVLSTEQQNELAGVLAHSQQRFLWVVRPPNDGIANATYFSVDGEIDPLKLLPEGFLEQTAGRGLVLPMWAPQIDVLSHESTGGFLTHCGWNSTLESVFHGVPLITWPLYAEQKMNAVMLTEGLRVGLRPSVGKDGIIRGAEIARVIGELMEGEEGKRIRSKMQELKRAASAVLSKDGSSTRALEEVAKIWESKV

SEQ ID NO: 3 - DNA, 1434 bp, synthetic construct
  source 1..1434: mol_type = other DNA
  CDS 1..1434: note = Mut87 - a Mutant UGT enzyme derived from PtUGT1
atgtctgaatcccccgccgctccaccaaccaccgctccaccaccgcacgtcataatcgtgccctccgccggcatgggccacctcatccccctcgccgagttcgccaagcgcctccttccgcgcttcaccttcaccttcgccgtacccaccagcggcccgccctcatcctcccagcgcgacttcctctcctccctccctgcctccatcgacacctccttcctccccccggtcgacctctccgacgccccctccgacgccaaaatcgagactctcatgtccctcatggttgtccgctccctcccctcgctccgcgacctcattgcctcctacgtggcctccggccgccgcgtcgccgccctcgtcgtcgaccttttcgccactgatgcaatcgacgtcgcccttgagctcggcatccgccctttcatcttcttcccctccaccgccatgaccctctccttcttcctccacctcgagaagcttgatgaaacggtgtcatgtgagtttgccgagctgtccgacccggttcagatccccgggtgtattccggtccacggcaaggatttgctggacccggttcaggataggaagaacgacgcctacaagtggctcctccaccactccaagaggtataaattggccgagggtgttatcgtaaatagcttcgaggatttggaggggggaccgatcagggagcttttgcaccccgagccgggaaagccgcgggtttacccggtcggaccgctgattcaggccggttcgtgcgagaagggggcagctgcccggcctgagtgcttgaagtggttggaccagcagccacgtggatccgtcctattcgtgaatttcgggagtggtggggtcctcagtacggagcagcagaacgagcttgcactgggtctggcccacagccagcagaggttcctatgggtggttaggcctccaaacgacggcattgccaacgccacgtatttcagcgtcgacggggagatcgacccgttgaaactcctgcccgaggggttcttggagcagaccgcgggcaggggtttggtcttgccaatgtgggccccgcagatcgatgtcttaagccatgagtcgacgggcgggttcttgacgcattgtgggtggaattcaacactggagagcgtgttccatggggtaccactaattgcatggcccctctatgcagagcaaaagatgaacgctgttatgctaaccgagggcctgagggtgggactcagacccaaagtgggtaaggatggaatcatccgaggtgctgagatcgcacgagttataaaagagttgatggaaggtgaggaagggaaacgaatacggagtaagatgcaggagttgaagcgtgcggcttctgctgtattgagcaaagatggatcatctactcgagctcttgaagaggttgcaaaaatttgggaaagcaaggtt

SEQ ID NO: 4 - protein, 478 aa, synthetic construct
MSESPAAPPTTAPPPHVIIVPSAGMGHLIPLAEFAKRLLPRFTFTFAVPTSGPPSSSQRDFLSSLPASIDTSFLPPVDLSDAPSDAKIETLMSLMVVRSLPSLRDLIASYVASGRRVAALVVDLFATDAIDVALELGIRPFIFFPSTAMTLSFFLHLEKLDETVSCEFAELSDPVQIPGCIPVHGKDLLDPVQDRKNDAYKWLLHHSKRYKLAEGVIVNSFEDLEGGPIRELLHPEPGKPRVYPVGPLIQAGSCEKGAAARPECLKWLDQQPRGSVLFVNFGSGGVLSTEQQNELALGLAHSQQRFLWVVRPPNDGIANATYFSVDGEIDPLKLLPEGFLEQTAGRGLVLPMWAPQIDVLSHESTGGFLTHCGWNSTLESVFHGVPLIAWPLYAEQKMNAVMLTEGLRVGLRPKVGKDGIIRGAEIARVIKELMEGEEGKRIRSKMQELKRAASAVLSKDGSSTRALEEVAKIWESKV

SEQ ID NO: 5 - DNA, 1434 bp, synthetic construct
  source 1..1434: mol_type = other DNA
  CDS 1..1434: note = Mut88 - a Mutant UGT enzyme derived from PtUGT1
atgtctgaatcccccgccgctccaccaaccaccgctccaccaccgcacgtcataatcgtgccctccgccggcatgggccacctcatccccctcgccgagttcgccaagcgcctccttccgcgcttcaccttcaccttcgccgtacccaccagcggcccgccctcatcctcccagcgcgacttcctctcctccctccctgcctccatcgacacctccttcctccccccggtcgacctctccgacgccccctccgacgccaaaatcgagactctcatgtccctcatggttgtccgctccctcccctcgctccgcgacctcattgcctcctacgtggcctccggccgccgcgtcgccgccctcgtcgtcgaccttttcgccactgatgcaatcgacgtcgcccttgagctcggcatccgccctttcatcttcttcccctccaccgccatgaccctctccttcttcctccacctcgagaagcttgatgaaacggtgtcatgtgagtttgccgagctgtccgacccggttcagatccccgggtgtattccggtccacggcaaggatttgctggacccggttcaggataggaagaacgacgcctacaagtggctcctccaccactccaagaggtataaattggccgagggtgttatcgtaaatagcttcgaggatttggaggggggaccgatcagggagcttttgcaccccgagccgggaaagccgcgggtttacccggtcggaccgctgattcaggccggttcgtgcgagaagggggcagctgcccggcctgagtgcttgaagtggttggaccagcagccacgtggatccgtcctattcgtgaatttcgggagtggtggggtcctcagtacggagcagcagaacgagcttgcactgggtctggcccacagccagcagaggttcctatgggtggttaggcctccaaacgacggcattgccaacgccacgtatttcagcgtcgacggggagatcgacccgttgaaactcctgcccgaggggttcttggagcagaccgcgggcaggggtttggtcttgccaatgtgggccccgcagatcgatgtcttaagccatgagtcgacgggcgggttcttgacgcattgtgggtggaattcaacactggagagcgtggtccatggggtaccactaattgcatggcccctctatgcagagcaaaagatgaacgctgttatgctaaccgagggcctgagggtgggactcagacccaaagtgggtaaggatggaatcatccgaggtgctgagatcgcacgagttataaaagagttgatggaaggtgaggaagggaaacgaatacggagtaagatgcaggagttgaagcgtgcggcttctgctgtattgagcaaagatggatcatctactcgagctcttgaagaggttgcaaaaatttgggaaagcaaggtt

SEQ ID NO: 6 - protein, 478 aa, synthetic construct
MSESPAAPPTTAPPPHVIIVPSAGMGHLIPLAEFAKRLLPRFTFTFAVPTSGPPSSSQRDFLSSLPASIDTSFLPPVDLSDAPSDAKIETLMSLMVVRSLPSLRDLIASYVASGRRVAALVVDLFATDAIDVALELGIRPFIFFPSTAMTLSFFLHLEKLDETVSCEFAELSDPVQIPGCIPVHGKDLLDPVQDRKNDAYKWLLHHSKRYKLAEGVIVNSFEDLEGGPIRELLHPEPGKPRVYPVGPLIQAGSCEKGAAARPECLKWLDQQPRGSVLFVNFGSGGVLSTEQQNELALGLAHSQQRFLWVVRPPNDGIANATYFSVDGEIDPLKLLPEGFLEQTAGRGLVLPMWAPQIDVLSHESTGGFLTHCGWNSTLESVVHGVPLIAWPLYAEQKMNAVMLTEGLRVGLRPKVGKDGIIRGAEIARVIKELMEGEEGKRIRSKMQELKRAASAVLSKDGSSTRALEEVAKIWESKV

SEQ ID NO: 7 - DNA, 1434 bp, synthetic construct
  source 1..1434: mol_type = other DNA
  CDS 1..1434: note = Mut90 - a Mutant UGT enzyme derived from PtUGT1
atgtctgaatcccccgccgctccaccaaccaccgctccaccaccgcacgtcataatcgtgccctccgccggcatgggccacctcatccccctcgccgagttcgccaagcgcctccttccgcgcttcaccttcaccttcgccgtacccaccagcggcccgccctcatcctcccagcgcgacttcctctcctccctccctgcctccatcgacacctccttcctccccccggtcgacctctccgacgccccctccgacgccaaaatcgagactctcatgtccctcatggttgtccgctccctcccctcgctccgcgacctcattgcctcctacgtggcctccggccgccgcgtcgccgccctcgtcgtcgaccttttcgccactgatgcaatcgacgtcgcccttgagctcggcatccgccctttcatcttcttcccctccaccgccatgaccctctccttcttcctccacctcgagaagcttgatgaaacggtgtcatgtgagtttgccgagctgtccgacccggttcagatccccgggtgtattccggtccacggcaaggatttgctggacccggttcaggataggaagaacgacgcctacaagtggctcctccaccactccaagaggtataaattggccgagggtgttatcgtaaatagcttcgaggatttggaggggggaccgatcagggagcttttgcaccccgagccgggaaagccgcgggtttacccggtcggaccgctgattcaggccggttcgtgcgagaagggggcagctgcccggcctgagtgcttgaagtggttggaccagcagccacgtggatccgtcctattcgtgaatttcgggagtggtggggtcctcagtacggagcagcagaacgagcttgcactgggtctggcccacagccagcagaggttcctatgggtggttaggcctccaaacgacggcattgccaacgccacgtatttcagcgtcgacggggagatcgacccgttgaaactcctgcccgaggggttcttggagcagaccgcgggcaggggtttggtcttgccaatgtgggccccgcagatcgatgtcttaagccatgagtcgacgggcgggttcttgacgcattgtgggtggaattcaacactggagagcgtggtccatggggtaccactaatttgctggcccctctatgcagagcaaaagatgaactgcgttatgctaaccgagggcctgagggtgggactcagacccaaagtgggtaaggatggaatcatccgaggtgctgagatcgcacgagttataaaagagttgatggaaggtgaggaagggaaacgaatacggagtaagatgcaggagttgaagcgtgcggcttctgctgtattgagcaaagatggatcatctactcgagctcttgaagaggttgcaaaaatttgggaaagcaaggtt

SEQ ID NO: 8 - protein, 478 aa, synthetic construct
MSESPAAPPTTAPPPHVIIVPSAGMGHLIPLAEFAKRLLPRFTFTFAVPTSGPPSSSQRDFLSSLPASIDTSFLPPVDLSDAPSDAKIETLMSLMVVRSLPSLRDLIASYVASGRRVAALVVDLFATDAIDVALELGIRPFIFFPSTAMTLSFFLHLEKLDETVSCEFAELSDPVQIPGCIPVHGKDLLDPVQDRKNDAYKWLLHHSKRYKLAEGVIVNSFEDLEGGPIRELLHPEPGKPRVYPVGPLIQAGSCEKGAAARPECLKWLDQQPRGSVLFVNFGSGGVLSTEQQNELALGLAHSQQRFLWVVRPPNDGIANATYFSVDGEIDPLKLLPEGFLEQTAGRGLVLPMWAPQIDVLSHESTGGFLTHCGWNSTLESVVHGVPLICWPLYAEQKMNCVMLTEGLRVGLRPKVGKDGIIRGAEIARVIKELMEGEEGKRIRSKMQELKRAASAVLSKDGSSTRALEEVAKIWESKV

SEQ ID NO: 9 - protein, 520 aa, Secale cereale
  REGION 1..520: note = peptide - Beta-glucosidase from Secale cereale
MAGTPSKPSEPIGPVFTKLKPWQIPKRDWFSKDFLFGASTSAYQIEGAWNEDGKGPSTWDHFCHTYPERISDGTNGDVAANSYHMYEEDVKALKDMGMKVYRFSISWSRILPNGTGKPNQKGIDYYNNLINSLIRHGIVPYVTIWHWDTPQALEDKYGGFLDKQIVNDYKYFAELCFQSFGDRVKNWFTFNEPHTYCCFSYGEGIHAPGRCSPGLDCAVPEGDSLREPYTAGHHILLAHAEAVELFKAHYNKHGDSKIGMAFDVMGYEPYQDSFLDDQARERSIDYNMGWFLEPVVRGDYPFSMRSLIGDRLPMFTKEEQEKLASSCDIMGLNYYTSRFSKHVDISSDYTPTLNTDDAYASSETTGSDGNEIGPITGTYWIYMYPKGLTDLLLIMKEKYGNPPIFITENGIADVEGDPEMPDPLDDWKRLDYLQRHISAVKDAIDQGADVRGHFTWGLIDNFEWGSGYSSRFGLVYIDKEDGNKRKLKKSAKWFAKFNSVPKTLLKTTNNNATVTASVSV

SEQ ID NO: 10 - protein, 431 aa, Thermus thermophilus
  REGION 1..431: note = peptide - BETA GLYCOSIDASE from THERMUS THERMOPHILUS
MTENAEKFLWGVATSAYQIEGATQEDGRGPSIWDAFAQRPGAIRDGSTGEPACDHYRRYEEDIALMQSLGVRAYRFSVAWPRILPEGRGRINPKGLAFYDRLVDRLLASGITPFLTLYHWDLPLALEERGGWRSRETAFAFAEYAEAVARALADRVPFFATLNEPWCSAFLGHWTGEHAPGLRNLEAALRAAHHLLLGHGLAVEALRAAGARRVGIVLNFAPAYGEDPEAVDVADRYHNRFFLDPILGKGYPESPFRDPPPVPILSRDLELVARPLDFLGVNYYAPVRVAPGTGTLPVRYLPPEGPATAMGWEVYPEGLHHLLKRLGREVPWPLYVTENGAAYPDLWTGEAVVEDPERVAYLEAHVEAALRAREEGVDLRGYFVWSLMDNFEWAFGYTRRFGLYYVDFPSQRRIPKRSALWYRERIARAQT

SEQ ID NO: 11 - protein, 446 aa, Thermotoga maritima
  REGION 1..446: note = peptide - BLG from THERMOTOGA MARITIMA - commercially available from Megazyme
MNVKKFPEGFLWGVATASYQIEGSPLADGAGMSIWHTFSHTPGNVKNGDTGDVACDHYNRWKEDIEIIEKLGVKAYRFSISWPRILPEGTGRVNQKGLDFYNRIIDTLLEKGITPFVTIYHWDLPFALQLKGGWANREIADWFAEYSRVLFENFGDRVKNWITLNEPWVVAIVGHLYGVHAPGMRDIYVAFRAVHNLLRAHARAVKVFRETVKDGKIGIVFNNGYFEPASEKEEDIRAVRFMHQFNNYPLFLNPIYRGDYPELVLEFAREYLPENYKDDMSEIQEKIDFVGLNYYSGHLVKFDPDAPAKVSFVERDLPKTAMGWEIVPEGIYWILKKVKEEYNPPEVYITENGAAFDDVVSEDGRVHDQNRIDYLKAHIGQAWKAIQEGVPLKGYFVWSLLDNFEWAEGYSKRFGIVYVDYSTQKRIVKDSGYWYSNVVKNNGLED

SEQ ID NO: 12 - DNA, 7150 bp, synthetic construct
  misc_feature 1..7150: note = Expression vector pTMH307
  regulatory 5408..5512: note = promoter - ApmR promoter; regulatory_class = promoter
  source 1..7150: mol_type = other DNA
  CDS 3471..4970: note = gene encoding PtUGT72B29 indoxyl glucosyltransferase from Polygonum tinctorium with N-terminal 6xHis tag
  CDS 5513..6373: note = bla gene encoding ApmR (amppicillin resistance protein)
tttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccttctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgaaacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggccatcaccatcaccatcacgactacgacatcccgactaccgaaaacctgtacttccagggttctgaatcccccgccgctccaccaaccaccgctccaccaccgcacgtcataatcgtgccctccgccggcatgggccacctcatccccctcgccgagttcgccaagcgcctccttccgcgcttcaccttcaccttcgccgtacccaccagcggcccgccctcatcctcccagcgcgacttcctctcctccctccctgcctccatcgacacctccttcctccccgaggtcgacctctccgacgccccctccgacgcccaaatcgagactctcatgtccctcatggttgtccgctccctcccctcgctccgcgacctcattgcctcctactccgcctccggccgccgcgtcgccgccctcgtcgtcgaccttttcgccactgatgcaatcgacgtcgcccttgagctcggcatccgccctttcatcttcttcccctccaccgccatgaccctctccttcttcctccacctcgagaagcttgatgaaacggtgtcatgtgagtttgccgagctgtccgacccggttcagatccccgggtgtattccggtccacggcaaggatttgatcgacccggttcaggataggaagaacgacgcctacaagtggctcctccaccactccaagaggtataaattggccgagggtgttatcgtaaatagcttcgagggtttggaggggggaccgatcagggagcttttgcaccccgagccgggaaagccgcgggtttacccggtcggaccgctgattcaggccggttcgtgcgagaagggggcagctgcccggcctgagtgcttgaagtggttggaccagcagccacgtggatccgtcctattcgtgaatttcgggagtggtggggtcctcagtacggagcagcagaacgagcttgcaggtgtgctggcccacagccagcagaggttcctatgggtggttaggcctccaaacgacggcattgccaacgccacgtatttcagcgtcgacggggagatcgacccgttgaaactcctgcccgaggggttcttggagcagaccgcgggcaggggtttggtcttgccaatgtgggccccgcagatcgatgtcttaagccatgagtcgacgggcgggttcttgacgcattgtgggtggaattcaacactggagagcgtgttccatggggtaccactaattacatggcccctctatgcagagcaaaagatgaacgctgttatgctaaccgagggcctgagggtgggactcagaccctcagtgggtaaggatggaatcatccgaggtgctgagatcgcacgagttataggggagttgatggaaggtgaggaagggaaacgaatacggagtaagatgcaggagttgaagcgtgcggcttctgctgtattgagcaaagatggatcatctactcgagctcttgaagaggttgcaaaaatttgggaaagcaaggtttaaggatcctgatcaatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcacgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcc

SEQ ID NO: 13 - protein, 499 aa, synthetic construct
MGHHHHHHDYDIPTTENLYFQGSESPAAPPTTAPPPHVIIVPSAGMGHLIPLAEFAKRLLPRFTFTFAVPTSGPPSSSQRDFLSSLPASIDTSFLPEVDLSDAPSDAQIETLMSLMVVRSLPSLRDLIASYSASGRRVAALVVDLFATDAIDVALELGIRPFIFFPSTAMTLSFFLHLEKLDETVSCEFAELSDPVQIPGCIPVHGKDLIDPVQDRKNDAYKWLLHHSKRYKLAEGVIVNSFEGLEGGPIRELLHPEPGKPRVYPVGPLIQAGSCEKGAAARPECLKWLDQQPRGSVLFVNFGSGGVLSTEQQNELAGVLAHSQQRFLWVVRPPNDGIANATYFSVDGEIDPLKLLPEGFLEQTAGRGLVLPMWAPQIDVLSHESTGGFLTHCGWNSTLESVFHGVPLITWPLYAEQKMNAVMLTEGLRVGLRPSVGKDGIIRGAEIARVIGELMEGEEGKRIRSKMQELKRAASAVLSKDGSSTRALEEVAKIWESKV

SEQ ID NO: 14 - protein, 286 aa, synthetic construct [sequence omitted]

SEQ ID NO: 15 - DNA, 31 bp, synthetic construct
  primer_bind 1..31: note = P14C forward
  source 1..31: mol_type = other DNA
  modified_base 9: mod_base = OTHER; note = uracil
atgccacgtcataatcgtgccctccgccggc

SEQ ID NO: 16 - DNA, 27 bp, synthetic construct
  primer_bind 1..27: note = P14C reverse
  source 1..27: mol_type = other DNA
  modified_base 9: mod_base = OTHER; note = uracil
acgtggcatggtggagcggtggttggt

SEQ ID NO: 17 - DNA, 28 bp, synthetic construct
  primer_bind 1..28: note = A117C forward
  source 1..28: mol_type = other DNA
  modified_base 7: mod_base = OTHER; note = uracil
accttttcgccactgatgcaatcgacgt

SEQ ID NO: 18 - DNA, 39 bp, synthetic construct
  primer_bind 1..39: note = A117C reverse
  source 1..39: mol_type = other DNA
  modified_base 7: mod_base = OTHER; note = uracil
aaaaggtcgacgacgagggcgcagacgcggcggccggag

SEQ ID NO: 19 - DNA, 32 bp, synthetic construct
  primer_bind 1..32: note = S21C forward
  source 1..32: mol_type = other DNA
  modified_base 10: mod_base = OTHER; note = uracil
atcgtgccctgcgccggcatgggccacctcat

SEQ ID NO: 20 - DNA, 28 bp, synthetic construct
  primer_bind 1..28: note = S21C reverse
  source 1..28: mol_type = other DNA
  modified_base 10: mod_base = OTHER; note = uracil
agggcacgattatgacgtgcggtggtgg

SEQ ID NO: 21 - DNA, 30 bp, synthetic construct
  primer_bind 1..30: note = D122C forward
  source 1..30: mol_type = other DNA
  modified_base 11: mod_base = OTHER; note = uracil
actgatgcaatcgacgtcgcccttgagctc

SEQ ID NO: 22 - DNA, 39 bp, synthetic construct
  primer_bind 1..39: note = D122C reverse
  source 1..39: mol_type = other DNA
  modified_base 11: mod_base = OTHER; note = uracil
attgcatcagtggcgaaaaggcagacgacgagggcggcg

SEQ ID NO: 23 - DNA, 59 bp, synthetic construct
  primer_bind 1..59: note = V76C / R97C forward
  source 1..59: mol_type = other DNA
  modified_base 10: mod_base = OTHER; note = uracil
acgcccaaatcgagactctcatgtccctcatggttgtctgctccctcccctcgctccgc

SEQ ID NO: 24 - DNA, 53 bp, synthetic construct
  primer_bind 1..53: note = V76C / R97C reverse
  source 1..53: mol_type = other DNA
  modified_base 10: mod_base = OTHER; note = uracil
atttgggcgtcggagggggcgtcggagaggtcgcactcggggaggaaggaggt

SEQ ID NO: 25 - DNA, 54 bp, synthetic construct
  primer_bind 1..54: note = L119C / A132C forward
  source 1..54: mol_type = other DNA
  modified_base 6: mod_base = OTHER; note = uracil
ccttttcgccactgatgcaatcgacgtctgccttgagctcggcatccgcccttt

SEQ ID NO: 26 - DNA, 34 bp, synthetic construct
  primer_bind 1..34: note = L119C / A132C reverse
  source 1..34: mol_type = other DNA
  modified_base 7: mod_base = OTHER; note = uracil
aaaaggtcgacgacgcaggcggcgacgcggcggc

SEQ ID NO: 27 - DNA, 33 bp, synthetic construct
  primer_bind 1..33: note = V121C / A125C forward
  source 1..33: mol_type = other DNA
  modified_base 7: mod_base = OTHER; note = uracil
accttttctgcactgatgcaatcgacgtcgccc

SEQ ID NO: 28 - DNA, 29 bp, synthetic construct
  primer_bind 1..29: note = V121C / A125C reverse
  source 1..29: mol_type = other DNA
  modified_base 7: mod_base = OTHER; note = uracil
aaaaggtcgcagacgagggcggcgacgcg

SEQ ID NO: 29 - DNA, 25 bp, synthetic construct
  primer_bind 1..25: note = T126C forward
  source 1..25: mol_type = other DNA
  modified_base 7: mod_base = OTHER; note = uracil
atgcaatcgacgtcgcccttgagct

SEQ ID NO: 30 - DNA, 29 bp, synthetic construct
  primer_bind 1..29: note = T126C reverse
  source 1..29: mol_type = other DNA
  modified_base 7: mod_base = OTHER; note = uracil
attgcatcgcaggcgaaaaggtcgacgac

SEQ ID NO: 31 - DNA, 33 bp, synthetic construct
  primer_bind 1..33: note = R208C forward
  source 1..33: mol_type = other DNA
  modified_base 9: mod_base = OTHER; note = uracil
aagtgctataaattggccgagggtgttatcgta

SEQ ID NO: 32 - DNA, 30 bp, synthetic construct
  primer_bind 1..30: note = R208C reverse
  source 1..30: mol_type = other DNA
  modified_base 9: mod_base = OTHER; note = uracil
atagcacttggagtggtggaggagccactt

SEQ ID NO: 33 - DNA, 35 bp, synthetic construct
  primer_bind 1..35: note = A132C / I137C forward
  source 1..35: mol_type = other DNA
  modified_base 9: mod_base = OTHER; note = uracil
agctcggctgccgccctttcatcttcttcccctcc

SEQ ID NO: 34 - DNA, 34 bp, synthetic construct
  primer_bind 1..34: note = A132C / I137C reverse
  source 1..34: mol_type = other DNA
  modified_base 9: mod_base = OTHER; note = uracil
agccgagctcaaggcagacgtcgattgcatcagt

SEQ ID NO: 35 - DNA, 41 bp, synthetic construct
  primer_bind 1..41: note = A132C / P139C forward
  source 1..41: mol_type = other DNA
  modified_base 10: mod_base = OTHER; note = uracil
agctcggcatccgctgcttcatcttcttcccctccaccgcc

SEQ ID NO: 36 - DNA, 35 bp, synthetic construct
  primer_bind 1..35: note = A132C / P139C reverse
  source 1..35: mol_type = other DNA
  modified_base 10: mod_base = OTHER; note = uracil
atgccgagctcaaggcagacgtcgattgcatcagt

SEQ ID NO: 37 - DNA, 26 bp, synthetic construct
  primer_bind 1..26: note = A147C forward
  source 1..26: mol_type = other DNA
  modified_base 8: mod_base = OTHER; note = uracil
acctgcatgaccctctccttcttcct

SEQ ID NO: 38 - DNA, 28 bp, synthetic construct
  primer_bind 1..28: note = A147C reverse
  source 1..28: mol_type = other DNA
  modified_base 8: mod_base = OTHER; note = uracil
atgcaggtggaggggaagaagatgaaag

SEQ ID NO: 39 - DNA, 32 bp, synthetic construct
  primer_bind 1..32: note = P227C forward
  source 1..32: mol_type = other DNA
  modified_base 9: mod_base = OTHER; note = uracil
aggggggatgcatcagggagcttttgcacccc

SEQ ID NO: 40 - DNA, 27 bp, synthetic construct
  primer_bind 1..27: note = P227C reverse
  source 1..27: mol_type = other DNA
  modified_base 9: mod_base = OTHER; note = uracil
atcccccctccaaaccctcgaagctat

SEQ ID NO: 41 - DNA, 42 bp, synthetic construct
  primer_bind 1..42: note = P173C / P181C forward
  source 1..42: mol_type = other DNA
  modified_base 10: mod_base = OTHER; note = uracil
atccccgggtgtatttgcgtccacggcaaggatttgatcgac

SEQ ID NO: 42 - DNA, 37 bp, synthetic construct
  primer_bind 1..37: note = P173C / P181C reverse
  source 1..37: mol_type = other DNA
  modified_base 10: mod_base = OTHER; note = uracil
acccggggatctgaacgcagtcggacagctcggcaaa

SEQ ID NO: 43 - DNA, 36 bp, synthetic construct
  primer_bind 1..36: note = A176C forward
  source 1..36: mol_type = other DNA
  modified_base 12: mod_base = OTHER; note = uracil
agtgccccgggtgtattccggtccacggcaaggatt

SEQ ID NO: 44 - DNA, 30 bp, synthetic construct
  primer_bind 1..30: note = I176C reverse
  source 1..30: mol_type = other DNA
  modified_base 12: mod_base = OTHER; note = uracil
acccggggcactgaaccgggtcggacagct

SEQ ID NO: 45 - DNA, 30 bp, synthetic construct
  primer_bind 1..30: note = D186C forward
  source 1..30: mol_type = other DNA
  modified_base 7: mod_base = OTHER; note = uracil
agtgcttgatcgacccggttcaggatagga

SEQ ID NO: 46 - DNA, 25 bp, synthetic construct
  primer_bind 1..25: note = D186C reverse
  source 1..25: mol_type = other DNA
  modified_base 7: mod_base = OTHER; note = uracil
aagcacttgccgtggaccggaatac

SEQ ID NO: 47 - DNA, 31 bp, synthetic construct
  primer_bind 1..31: note = K396C forward
  source 1..31: mol_type = other DNA
  modified_base 7: mod_base = OTHER; note = uracil
aatgcatgaacgctgttatgctaaccgaggg

SEQ ID NO: 48 - DNA, 29 bp, synthetic construct
  primer_bind 1..29: note = K396C reverse
  source 1..29: mol_type = other DNA
  modified_base 7: mod_base = OTHER; note = uracil
atgcattgctctgcatagaggggccatgt

SEQ ID NO: 49 - DNA, 35 bp, synthetic construct
  primer_bind 1..35: note = P190C / A198C forward
  source 1..35: mol_type = other DNA
  modified_base 9: mod_base = OTHER; note = uracil
agaacgactgctacaagtggctcctccaccactcc

SEQ ID NO: 50 - DNA, 43 bp, synthetic construct
  primer_bind 1..43: note = P190C / A198C reverse
  source 1..43: mol_type = other DNA
  modified_base 9: mod_base = OTHER; note = uracil
agtcgttcttcctatcctgaacgcagtcgatcaaatccttgcc

SEQ ID NO: 51 - DNA, 31 bp, synthetic construct
  primer_bind 1..31: note = D193C / N196C forward
  source 1..31: mol_type = other DNA
  modified_base 9: mod_base = OTHER; note = uracil
aggaagtgcgacgcctacaagtggctcctcc

SEQ ID NO: 52 - DNA, 28 bp, synthetic construct
  primer_bind 1..28: note = D193C / N196C reverse
  source 1..28: mol_type = other DNA
  modified_base 7: mod_base = OTHER; note = uracil
acttcctgcactgaaccgggtcgatcaa

SEQ ID NO: 53 - DNA, 36 bp, synthetic construct
  primer_bind 1..36: note = A259C / L264C fw
  source 1..36: mol_type = other DNA
  modified_base 12: mod_base = OTHER; note = uracil
agtgctgcaagtggttggaccagcagccacgtggat

SEQ ID NO: 54 - DNA, 40 bp, synthetic construct
  primer_bind 1..40: note = A259C / L264C reverse
  source 1..40: mol_type = other DNA
  modified_base 12: mod_base = OTHER; note = uracil
acttgcagcactcaggccggcaagctgcccccttctcgca

SEQ ID NO: 55 - DNA, 38 bp, synthetic construct
  primer_bind 1..38: note = P271C / S274C forward
  source 1..38: mol_type = other DNA
  modified_base 8: mod_base = OTHER; note = uracil
agtgccgtggatgcgtcctattcgtgaatttcgggagt

SEQ ID NO: 56 - DNA, 26 bp, synthetic construct
  primer_bind 1..26: note = P271C / S274C reverse
  source 1..26: mol_type = other DNA
  modified_base 8: mod_base = OTHER; note = uracil
acggcactgctggtccaaccacttca

SEQ ID NO: 57 - DNA, 26 bp, synthetic construct
  primer_bind 1..26: note = G273C forward
  source 1..26: mol_type = other DNA
  modified_base 8: mod_base = OTHER; note = uracil
acgttgctccgtcctattcgtgaatt

SEQ ID NO: 58 - DNA, 28 bp, synthetic construct
  primer_bind 1..28: note = G273C reverse
  source 1..28: mol_type = other DNA
  modified_base 8: mod_base = OTHER; note = uracil
agcaacgtggctgctggtccaaccactt

SEQ ID NO: 59 - DNA, 34 bp, synthetic construct
  primer_bind 1..34: note = G365C forward
  source 1..34: mol_type = other DNA
  modified_base 10: mod_base = OTHER; note = uracil
acgtgcgggttcttgacgcattgtgggtggaatt

SEQ ID NO: 60 - DNA, 28 bp, synthetic construct
  primer_bind 1..28: note = G365C reverse
  source 1..28: mol_type = other DNA
  modified_base 10: mod_base = OTHER; note = uracil
acccgcacgtcgactcatggcttaagac

SEQ ID NO: 61 - DNA, 28 bp, synthetic construct
  primer_bind 1..28: note = V278C forward
  source 1..28: mol_type = other DNA
  modified_base 10: mod_base = OTHER; note = uracil
attctgcaatttcgggagtggtggggtc

SEQ ID NO: 62 - DNA, 28 bp, synthetic construct
  primer_bind 1..28: note = V278C reverse
  source 1..28: mol_type = other DNA
  modified_base 10: mod_base = OTHER; note = uracil
attgcagaataggacggatccacgtggc

SEQ ID NO: 63 - DNA, 32 bp, synthetic construct
  primer_bind 1..32: note = W307C forward
  source 1..32: mol_type = other DNA
  modified_base 9: mod_base = OTHER; note = uracil
atgcgtggttaggcctccaaacgacggcattg

SEQ ID NO: 64 - DNA, 27 bp, synthetic construct
  primer_bind 1..27: note = W307C reverse
  source 1..27: mol_type = other DNA
  modified_base 9: mod_base = OTHER; note = uracil
accacgcataggaacctctgctggctg

SEQ ID NO: 65 - DNA, 34 bp, synthetic construct
  primer_bind 1..34: note = L286C / Q290C forward
  source 1..34: mol_type = other DNA
  modified_base 10: mod_base = OTHER; note = uracil
agtacggagtgccagaacgagcttgcaggtgtgc

SEQ ID NO: 66 - DNA, 32 bp, synthetic construct
  primer_bind 1..32: note = L286C / Q290C reverse
  source 1..32: mol_type = other DNA
  modified_base 10: mod_base = OTHER; note = uracil
actccgtactgcagaccccaccactcccgaaa

SEQ ID NO: 67 - DNA, 34 bp, synthetic construct
  primer_bind 1..34: note = G337C / E340C forward
  source 1..34: mol_type = other DNA
  modified_base 9: mod_base = OTHER; note = uracil
agtgcttcttgtgccagaccgcgggcaggggttt

SEQ ID NO: 68 - DNA, 27 bp, synthetic construct
  primer_bind 1..27: note = G337C / E340C reverse
  source 1..27: mol_type = other DNA
  modified_base 9: mod_base = OTHER; note = uracil
agaagcactcgggcaggagtttcaacg

SEQ ID NO: 69 - DNA, 29 bp, synthetic construct
  primer_bind 1..29: note = T342C / G346C forward
  source 1..29: mol_type = other DNA
  modified_base 7: mod_base = OTHER; note = uracil
aggtgcttggtcttgccaatgtgggcccc

SEQ ID NO: 70 - DNA, 34 bp, synthetic construct
  primer_bind 1..34: note = T342C / G346C reverse
  source 1..34: mol_type = other DNA
  modified_base 7: mod_base = OTHER; note = uracil
agcacctgcccgcgcactgctccaagaacccctc

SEQ ID NO: 71 - DNA, 53 bp, synthetic construct
  primer_bind 1..53: note = L368C / I387C forward
  source 1..53: mol_type = other DNA
  modified_base 7: mod_base = OTHER; note = uracil
agagcgtgttccatggggtaccactatgcacatggcccctctatgcagagcaa

SEQ ID NO: 72 - DNA, 57 bp, synthetic construct
  primer_bind 1..57: note = L368C / I387C reverse
  source 1..57: mol_type = other DNA
  modified_base 7: mod_base = OTHER; note = uracil
acgctctccagtgttgaattccacccacaatgcgtgcagaacccgcccgtcgactca

SEQ ID NO: 73 - DNA, 41 bp, synthetic construct
  primer_bind 1..41: note = T388C / A399C forward
  source 1..41: mol_type = other DNA
  modified_base 10: mod_base = OTHER; note = uracil
agcaaaagatgaactgcgttatgctaaccgagggcctgagg

SEQ ID NO: 74 - DNA, 52 bp, synthetic construct
  primer_bind 1..52: note = T388C / A399C reverse
  source 1..52: mol_type = other DNA
  modified_base 10: mod_base = OTHER; note = uracil
atcttttgctctgcatagaggggccagcaaattagtggtaccccatggaaca

SEQ ID NO: 75 - DNA, 47 bp, synthetic construct
  primer_bind 1..47: note = P412C / E424C forward
  source 1..47: mol_type = other DNA
  modified_base 7: mod_base = OTHER; note = uracil
atggaatcatccgaggtgcttgcatcgcacgagttataggggagttg

SEQ ID NO: 76 - DNA, 41 bp, synthetic construct
  primer_bind 1..41: note = P412C / E424C reverse
  source 1..41: mol_type = other DNA
  modified_base 7: mod_base = OTHER; note = uracil
attccatccttacccactgagcatctgagtcccaccctcag

SEQ ID NO: 77 - DNA, 42 bp, synthetic construct
  primer_bind 1..42: note = V455C / S462C forward
  source 1..42: mol_type = other DNA
  modified_base 9: mod_base = OTHER; note = uracil
agcaaagatggatcatgcactcgagctcttgaagaggttgca

SEQ ID NO: 78 - DNA, 33 bp, synthetic construct
  primer_bind 1..33: note = V455C / S462C reverse
  source 1..33: mol_type = other DNA
  modified_base 9: mod_base = OTHER; note = uracil
atctttgctcaagcaagcagaagccgcacgctt

SEQ ID NO: 79 - DNA, 33 bp, synthetic construct
  primer_bind 1..33: note = S457C / G460C forward
  source 1..33: mol_type = other DNA
  modified_base 7: mod_base = OTHER; note = uracil
aaagattgctcatctactcgagctcttgaagag

SEQ ID NO: 80 - DNA, 28 bp, synthetic construct
  primer_bind 1..28: note = S457C / G460C reverse
  source 1..28: mol_type = other DNA
  modified_base 7: mod_base = OTHER; note = uracil
aatctttgcacaatacagcagaagccgc

SEQ ID NO: 81 - DNA, 30 bp, synthetic construct
  primer_bind 1..30: note = L64C / I68C forward
  source 1..30: mol_type = other DNA
  modified_base 9: mod_base = OTHER; note = uracil
acacctccttcctccccgaggtcgacctct

SEQ ID NO: 82 - DNA, 43 bp, synthetic construct
  primer_bind 1..43: note = L64C / I68C reverse
  source 1..43: mol_type = other DNA
  modified_base 9: mod_base = OTHER; note = uracil
aggaggtgtcgcaggaggcagggcaggaggagaggaagtcgcg

SEQ ID NO: 83 - DNA, 31 bp, synthetic construct
  primer_bind 1..31: note = T146C/M148C forward
  source 1..31: mol_type = other DNA
  modified_base 7: mod_base = OTHER; note = uracil
accctctccttcttcctccacctcgagaagc

SEQ ID NO: 84 - DNA, 36 bp, synthetic construct
  primer_bind 1..36: note = T146C/M148C reverse
  source 1..36: mol_type = other DNA
  modified_base 7: mod_base = OTHER; note = uracil
agagggtgcaggcgcaggaggggaagaagatgaaag

SEQ ID NO: 85 - DNA, 33 bp, synthetic construct
  primer_bind 1..33: note = E224C forward
  source 1..33: mol_type = other DNA
  modified_base 9: mod_base = OTHER; note = uracil
agggtttgtgcgggggaccgatcagggagcttt

SEQ ID NO: 86 - DNA, 29 bp, synthetic construct
  primer_bind 1..29: note = E224C reverse
  source 1..29: mol_type = other DNA
  modified_base 9: mod_base = OTHER; note = uracil
acaaaccctcgaagctatttacgataaca

SEQ ID NO: 87 - DNA, 34 bp, synthetic construct
  primer_bind 1..34: note = E235C / K238C forward
  source 1..34: mol_type = other DNA
  modified_base 12: mod_base = OTHER; note = uracil
atgcccgcgggtttacccggtcggaccgctgatt

SEQ ID NO: 88 - DNA, 41 bp, synthetic construct
  primer_bind 1..41: note = E235C / K238C reverse
  source 1..41: mol_type = other DNA
  modified_base 12: mod_base = OTHER; note = uracil
acccgcgggcatcccgggcaggggtgcaaaagctccctgat

SEQ ID NO: 89 - DNA, 27 bp, synthetic construct
  primer_bind 1..27: note = L267C forward
  source 1..27: mol_type = other DNA
  modified_base 6: mod_base = OTHER; note = uracil
agtggtgcgaccagcagccacgtggat

SEQ ID NO: 90 - DNA, 25 bp, synthetic construct
  primer_bind 1..25: note = L267C reverse
  source 1..25: mol_type = other DNA
  modified_base 7: mod_base = OTHER; note = uracil
accacttcaagcactcaggccgggc

SEQ ID NO: 91 - DNA, 34 bp, synthetic construct
  primer_bind 1..34: note = S363C forward
  source 1..34: mol_type = other DNA
  modified_base 10: mod_base = OTHER; note = uracil
agccatgagtgcacgggcgggttcttgacgcatt

SEQ ID NO: 92 - DNA, 30 bp, synthetic construct
  primer_bind 1..30: note = S363C reverse
  source 1..30: mol_type = other DNA
  modified_base 10: mod_base = OTHER; note = uracil
actcatggcttaagacatcgatctgcgggg

SEQ ID NO: 93 - DNA, 27 bp, synthetic construct
  primer_bind 1..27: note = N279C forward
  source 1..27: mol_type = other DNA
  modified_base 7: mod_base = OTHER; note = uracil
ttcgtgtgcttcgggagtggtggggtc

SEQ ID NO: 94 - DNA, 26 bp, synthetic construct
  primer_bind 1..26: note = N279C reverse
  source 1..26: mol_type = other DNA
  modified_base 8: mod_base = OTHER; note = uracil
acacgaataggacggatccacgtggc

SEQ ID NO: 95 - DNA, 32 bp, synthetic construct
  primer_bind 1..32: note = V308C forward
  source 1..32: mol_type = other DNA
  modified_base 9: mod_base = OTHER; note = uracil
atggtgcgttaggcctccaaacgacggcattg

SEQ ID NO: 96 - DNA, 27 bp, synthetic construct
  primer_bind 1..27: note = V308C reverse
  source 1..27: mol_type = other DNA
  modified_base 9: mod_base = OTHER; note = uracil
acgcaccataggaacctctgctggctg

SEQ ID NO: 97 - DNA, 35 bp, synthetic construct
  primer_bind 1..35: note = P390C/Q395C forward
  source 1..35: mol_type = other DNA
  modified_base 9: mod_base = OTHER; note = uracil
atgcagagtgcaagatgaacgctgttatgctaacc

SEQ ID NO: 98 - DNA, 36 bp, synthetic construct
  primer_bind 1..36: note = P390C/Q395C reverse
  source 1..36: mol_type = other DNA
  modified_base 9: mod_base = OTHER; note = uracil
actctgcatagaggcaccatgtaattagtggtaccc

SEQ ID NO: 99 - DNA, 37 bp, synthetic construct
  primer_bind 1..37: note = G460C/T463C forward
  source 1..37: mol_type = other DNA
  modified_base 9: mod_base = OTHER; note = uracil
attgctcatcttgccgagctcttgaagaggttgcaaa

SEQ ID NO: 100 - DNA, 29 bp, synthetic construct
  primer_bind 1..29: note = G460C/T463C reverse
  source 1..29: mol_type = other DNA
  modified_base 9: mod_base = OTHER; note = uracil
atgagcaatctttgctcaatacagcagaa

SEQ ID NO: 101 - DNA, 33 bp, synthetic construct
  primer_bind 1..33: note = P48C forward
  source 1..33: mol_type = other DNA
  modified_base 11: mod_base = OTHER; note = uracil
accttcgccgtatgcaccagcggcccgccctca

SEQ ID NO: 102 - DNA, 29 bp, synthetic construct
  primer_bind 1..29: note = P48C reverse
  source 1..29: mol_type = other DNA
  modified_base 11: mod_base = OTHER; note = uracil
acggcgaaggtgaaggtgaagcgcggaag

SEQ ID NO: 103 - DNA, 38 bp, synthetic construct
  primer_bind 1..38: note = S98C forward
  source 1..38: mol_type = other DNA
  modified_base 8: mod_base = OTHER; note = uracil
atggttgtccgctgcctcccctcgctccgcgacctcat

SEQ ID NO: 104 - DNA, 31 bp, synthetic construct
  primer_bind 1..31: note = S98C reverse
  source 1..31: mol_type = other DNA
  modified_base 7: mod_base = OTHER; note = uracil
caaccatgagggacatgagagtctcgatttg

SEQ ID NO: 105 - DNA, 30 bp, synthetic construct
  primer_bind 1..30: note = P13C forward
  source 1..30: mol_type = other DNA
  modified_base 10: mod_base = OTHER; note = uracil
accgctccatgcccgcacgtcataatcgtg

SEQ ID NO: 106 - DNA, 28 bp, synthetic construct
  primer_bind 1..28: note = P13C reverse
  source 1..28: mol_type = other DNA
  modified_base 10: mod_base = OTHER; note = uracil
atggagcggtggttggtggagcggcggg

SEQ ID NO: 107 - DNA, 31 bp, synthetic construct
  primer_bind 1..31: note = A66C forward
  source 1..31: mol_type = other DNA
  modified_base 10: mod_base = OTHER; note = uracil
atcgacacctccttcctccccgaggtcgacc

SEQ ID NO: 108 - DNA, 34 bp, synthetic construct
  primer_bind 1..34: note = A66C reverse
  source 1..34: mol_type = other DNA
  modified_base 10: mod_base = OTHER; note = uracil
aggtgtcgatggagcaagggagggaggagaggaa

SEQ ID NO: 109 - DNA, 27 bp, synthetic construct
  primer_bind 1..27: note = T10C forward
  source 1..27: mol_type = other DNA
  modified_base 9: mod_base = OTHER; note = uracil
acctgcgctccaccaccgcacgtcata

SEQ ID NO: 110 - DNA, 27 bp, synthetic construct
  primer_bind 1..27: note = T10C reverse
  source 1..27: mol_type = other DNA
  modified_base 9: mod_base = OTHER; note = uracil
agcgcaggttggtggagcggcggggga

SEQ ID NO: 111 - DNA, 29 bp, synthetic construct
  primer_bind 1..29: note = S112C forward
  source 1..29: mol_type = other DNA
  modified_base 9: mod_base = OTHER; note = uracil
actccgcctgcggccgccgcgtcgccgcc

SEQ ID NO: 112 - DNA, 27 bp, synthetic construct
  primer_bind 1..27: note = S112C reverse
  source 1..27: mol_type = other DNA
  modified_base 9: mod_base = OTHER; note = uracil
aggcggagtaggaggcaatgaggtcgc

SEQ ID NO: 113 - DNA, 33 bp, synthetic construct
  primer_bind 1..33: note = A22P forward
  source 1..33: mol_type = other DNA
  modified_base 10: mod_base = OTHER; note = uracil
atcgtgccctccccgggcatgggccacctcatc

SEQ ID NO: 114 - DNA, 28 bp, synthetic construct
  primer_bind 1..28: note = A22P reverse
  source 1..28: mol_type = other DNA
  modified_base 10: mod_base = OTHER; note = uracil
agggcacgattatgacgtgcggtggtgg

SEQ ID NO: 115 - DNA, 30 bp, synthetic construct
  primer_bind 1..30: note = M91I forward
  source 1..30: mol_type = other DNA
  modified_base 8: mod_base = OTHER; note = uracil
atctccctcatggttgtccgctccctcccc

SEQ ID NO: 116 - DNA, 27 bp, synthetic construct
  primer_bind 1..27: note = M91I reverse
  source 1..27: mol_type = other DNA
  modified_base 8: mod_base = OTHER; note = uracil
agggagatgagagtctcgatttgggcg

SEQ ID NO: 117 - DNA, 38 bp, synthetic construct
  primer_bind 1..38: note = E75P forward
  source 1..38: mol_type = other DNA
  modified_base 8: mod_base = OTHER; note = uracil
acctccttcctccccccggtcgacctctccgacgcccc

SEQ ID NO: 118 - DNA, 26 bp, synthetic construct
  primer_bind 1..26: note = E75P reverse
  source 1..26: mol_type = other DNA
  modified_base 8: mod_base = OTHER; note = uracil
aaggaggtgtcgatggaggcagggag

SEQ ID NO: 119 - DNA, 37 bp, synthetic construct
  primer_bind 1..37: note = E157P forward
  source 1..37: mol_type = other DNA
  modified_base 13: mod_base = OTHER; note = uracil
acctcccgaagcttgatgaaacggtgtcatgtgagtt

SEQ ID NO: 120 - DNA, 35 bp, synthetic construct
  primer_bind 1..35: note = E157P reverse
  source 1..35: mol_type = other DNA
  modified_base 13: mod_base = OTHER; note = uracil
agcttcgggaggtggaggaagaaggagagggtcat

SEQ ID NO: 121 - DNA, 27 bp, synthetic construct
  primer_bind 1..27: note = G222D forward
  source 1..27: mol_type = other DNA
  modified_base 7: mod_base = OTHER; note = uracil
aggatttggaggggggaccgatcaggg

SEQ ID NO: 122 - DNA, 27 bp, synthetic construct
  primer_bind 1..27: note = G222D reverse
  source 1..27: mol_type = other DNA
  modified_base 7: mod_base = OTHER; note = uracil
aaatcctcgaagctatttacgataaca

SEQ ID NO: 123 - DNA, 31 bp, synthetic construct
  primer_bind 1..31: note = G405D forward
  source 1..31: mol_type = other DNA
  modified_base 7: mod_base = OTHER; note = uracil
aggacctgagggtgggactcagaccctcagt

SEQ ID NO: 124 - DNA, 28 bp, synthetic construct
  primer_bind 1..28: note = G405D reverse
  source 1..28: mol_type = other DNA
  modified_base 7: mod_base = OTHER; note = uracil
aggtcctcggttagcataacagcgttca

SEQ ID NO: 125 - DNA, 35 bp, synthetic construct
  primer_bind 1..35: note = G409A forward
  source 1..35: mol_type = other DNA
  modified_base 11: mod_base = OTHER; note = uracil
agggtggcactcagaccctcagtgggtaaggatgg

SEQ ID NO: 126 - DNA, 29 bp, synthetic construct
  primer_bind 1..29: note = G409 reverse
  source 1..29: mol_type = other DNA
  modified_base 11: mod_base = OTHER; note = uracil
agtgccaccctcaggccctcggttagcat

SEQ ID NO: 127 - DNA, 34 bp, synthetic construct
  primer_bind 1..34: note = G430K forward
  source 1..34: mol_type = other DNA
  modified_base 10: mod_base = OTHER; note = uracil
ataaaagagttgatggaaggtgaggaagggaaac

SEQ ID NO: 128 - DNA, 28 bp, synthetic construct
  primer_bind 1..28: note = G430K reverse
  source 1..28: mol_type = other DNA
  modified_base 10: mod_base = OTHER; note = uracil
actcttttataactcgtgcgatctcagc

SEQ ID NO: 129 - DNA, 25 bp, synthetic construct
  primer_bind 1..25: note = G222D (45) forward
  source 1..25: mol_type = other DNA
  modified_base 7: mod_base = OTHER; note = uracil
aggatttggaggggggaccgatcag

SEQ ID NO: 130 - DNA, 27 bp, synthetic construct
  primer_bind 1..27: note = G222D (45) reverse
  source 1..27: mol_type = other DNA
  modified_base 7: mod_base = OTHER; note = uracil
aaatcctcgaagctatttacgataaca

SEQ ID NO: 131 - DNA, 68 bp, synthetic construct
  primer_bind 1..68: note = G405D / G409A / G430K forward
  source 1..68: mol_type = other DNA
  modified_base 10: mod_base = OTHER; note = uracil
aggatggaatcatccgaggtgctgagatcgcacgagttataaaagagttgatggaaggtgaggaaggg

SEQ ID NO: 132 - DNA, 63 bp, synthetic construct
  primer_bind 1..63: note = G405D / G409A / G430K reverse
  source 1..63: mol_type = other DNA
  modified_base 10: mod_base = OTHER; note = uracil
attccatccttacccactgagggtctgagtgccaccctcaggtcctcggttagcataacagcg

SEQ ID NO: 133 - DNA, 36 bp, synthetic construct
  primer_bind 1..36: note = E75P (46) forward
  source 1..36: mol_type = other DNA
  modified_base 8: mod_base = OTHER; note = uracil
acctccttcctccccccggtcgacctctccgacgcc

SEQ ID NO: 134 - DNA, 26 bp, synthetic construct
  primer_bind 1..26: note = E75P (46) reverse
  source 1..26: mol_type = other DNA
  modified_base 8: mod_base = OTHER; note = uracil
aaggaggtgtcgatggaggcagggag

SEQ ID NO: 135 - DNA, 31 bp, synthetic construct
  primer_bind 1..31: note = E157P (46) forward
  source 1..31: mol_type = other DNA
  modified_base 13: mod_base = OTHER; note = uracil
acctcccgaagcttgatgaaacggtgtcatg

SEQ ID NO: 136 - DNA, 34 bp, synthetic construct
  primer_bind 1..34: note = E157P (46) reverse
  source 1..34: mol_type = other DNA
  modified_base 13: mod_base = OTHER; note = uracil
agcttcgggaggtggaggaagaaggagagggtca

SEQ ID NO: 137 - DNA, 25 bp, synthetic construct
  primer_bind 1..25: note = G222D (46) forward
  source 1..25: mol_type = other DNA
  modified_base 7: mod_base = OTHER; note = uracil
aggatttggaggggggaccgatcag

SEQ ID NO: 138 - DNA, 26 bp, synthetic construct
  primer_bind 1..26: note = G222D (46) reverse
  source 1..26: mol_type = other DNA
  modified_base 7: mod_base = OTHER; note = uracil
aaatcctcgaagctatttacgataac

SEQ ID NO: 139 - DNA, 31 bp, synthetic construct
  primer_bind 1..31: note = S50D forward
  source 1..31: mol_type = other DNA
  modified_base 12: mod_base = OTHER; note = uracil
acggcccgccctcatcctcccagcgcgactt

SEQ ID NO: 140 - DNA, 31 bp, synthetic construct
  primer_bind 1..31: note = S50D reverse
  source 1..31: mol_type = other DNA
  modified_base 12: mod_base = OTHER; note = uracil
agggcgggccgtcggtgggtacggcgaaggt

SEQ ID NO: 141 - DNA, 26 bp, synthetic construct
  primer_bind 1..26: note = M94T forward
  source 1..26: mol_type = other DNA
  modified_base 8: mod_base = OTHER; note = uracil
acggttgtccgctccctcccctcgct

SEQ ID NO: 142 - DNA, 31 bp, synthetic construct
  primer_bind 1..31: note = M94T reverse
  source 1..31: mol_type = other DNA
  modified_base 8: mod_base = OTHER; note = uracil
acaaccgtgagggacatgagagtctcgattt

SEQ ID NO: 143 - DNA, 34 bp, synthetic construct
  primer_bind 1..34: note = Q86K forward
  source 1..34: mol_type = other DNA
  modified_base 10: mod_base = OTHER; note = uracil
acgccaaaatcgagactctcatgtccctcatggt

SEQ ID NO: 144 - DNA, 28 bp, synthetic construct
  primer_bind 1..28: note = Q86K reverse
  source 1..28: mol_type = other DNA
  modified_base 10: mod_base = OTHER; note = uracil
attttggcgtcggagggggcgtcggaga

SEQ ID NO: 145 - DNA, 32 bp, synthetic construct
  primer_bind 1..32: note = Q86R forward
  source 1..32: mol_type = other DNA
  modified_base 8: mod_base = OTHER; note = uracil
acgcccgtatcgagactctcatgtccctcatg

SEQ ID NO: 146 - DNA, 26 bp, synthetic construct
  primer_bind 1..26: note = Q86R reverse
  source 1..26: mol_type = other DNA
  modified_base 8: mod_base = OTHER; note = uracil
acgggcgtcggagggggcgtcggaga

SEQ ID NO: 147 - DNA, 26 bp, synthetic construct
  primer_bind 1..26: note = A107K forward
  source 1..26: mol_type = other DNA
  modified_base 7: mod_base = OTHER; note = uracil
attaaatcctactccgcctccggccg

SEQ ID NO: 148 - DNA, 25 bp, synthetic construct
  primer_bind 1..25: note = A107K reverse
  source 1..25: mol_type = other DNA
  modified_base 7: mod_base = OTHER; note = uracil
atttaatgaggtcgcggagcgaggg

SEQ ID NO: 149 - DNA, 31 bp, synthetic construct
  primer_bind 1..31: note = K210R forward
  source 1..31: mol_type = other DNA
  modified_base 7: mod_base = OTHER; note = uracil
atcgtttggccgagggtgttatcgtaaatag

SEQ ID NO: 150 - DNA, 25 bp, synthetic construct
  primer_bind 1..25: note = K210R reverse
  source 1..25: mol_type = other DNA
  modified_base 7: mod_base = OTHER; note = uracil
aaacgatacctcttggagtggtgga

SEQ ID NO: 151 - DNA, 32 bp, synthetic construct
  primer_bind 1..32: note = Q269E forward
  source 1..32: mol_type = other DNA
  modified_base 8: mod_base = OTHER; note = uracil
agccacgtggatccgtcctattcgtgaatttc

SEQ ID NO: 152 - DNA, 30 bp, synthetic construct
  primer_bind 1..30: note = Q269E reverse
  source 1..30: mol_type = other DNA
  modified_base 8: mod_base = OTHER; note = uracil
acgtggctgctcgtccaaccacttcaagca

SEQ ID NO: 153 - DNA, 30 bp, synthetic construct
  primer_bind 1..30: note = V285T forward
  source 1..30: mol_type = other DNA
  modified_base 9: mod_base = OTHER; note = uracil
accctcagtacggagcagcagaacgagctt

SEQ ID NO: 154 - DNA, 27 bp, synthetic construct
  primer_bind 1..27: note = V285T reverse
  source 1..27: mol_type = other DNA
  modified_base 9: mod_base = OTHER; note = uracil
actgagggtcccaccactcccgaaatt

SEQ ID NO: 155 - DNA, 31 bp, synthetic construct
  primer_bind 1..31: note = A299E forward
  source 1..31: mol_type = other DNA
  modified_base 9: mod_base = OTHER; note = uracil
aggtgtgctggaacacagccagcagaggttc

SEQ ID NO: 156 - DNA, 27 bp, synthetic construct
  primer_bind 1..27: note = A299E reverse
  source 1..27: mol_type = other DNA
  modified_base 9: mod_base = OTHER; note = uracil
agcacacctgcaagctcgttctgctgc

SEQ ID NO: 157 - DNA, 36 bp, synthetic construct
  primer_bind 1..36: note = Q341R forward
  source 1..36: mol_type = other DNA
  modified_base 9: mod_base = OTHER; note = uracil
aggggttcttggagcgtaccgcgggcaggggtttgg

SEQ ID NO: 158 - DNA, 27 bp, synthetic construct
  primer_bind 1..27: note = Q341R reverse
  source 1..27: mol_type = other DNA
  modified_base 9: mod_base = OTHER; note = uracil
agaacccctcgggcaggagtttcaacg

SEQ ID NO: 159 - DNA, 46 bp, synthetic construct
  primer_bind 1..46: note = K332D / E336K / Q341R / A343K forward
  source 1..46: mol_type = other DNA
  modified_base 8: mod_base = OTHER; note = uracil
agggttcttggagcgtaccaaaggcaggggtttggtcttgccaatg

SEQ ID NO: 160 - DNA, 40 bp, synthetic construct
  primer_bind 1..40: note = K332D / E336K / Q341R / A343K reverse
  source 1..40: mol_type = other DNA
  modified_base 8: mod_base = OTHER; note = uracil
agaaccctttgggcaggagatccaacgggtcgatctcccc

SEQ ID NO: 161 - DNA, 33 bp, synthetic construct
  primer_bind 1..33: note = S413K forward
  source 1..33: mol_type = other DNA
  modified_base 9: mod_base = OTHER; note = uracil
acccaaagtgggtaaggatggaatcatccgagg

SEQ ID NO: 162 - DNA, 27 bp, synthetic construct
  primer_bind 1..27: note = S413K reverse
  source 1..27: mol_type = other DNA
  modified_base 9: mod_base = OTHER; note = uracil
actttgggtctgagtcccaccctcagg

SEQ ID NO: 163 - DNA, 31 bp, synthetic construct
  primer_bind 1..31: note = I473K forward
  source 1..31: mol_type = other DNA
  modified_base 7: mod_base = OTHER; note = uracil
aaaaaatgggaaagcaaggtttaaggatcct

SEQ ID NO: 164 - DNA, 25 bp, synthetic construct
  primer_bind 1..25: note = I472K reverse
  source 1..25: mol_type = other DNA
  modified_base 7: mod_base = OTHER; note = uracil
atttttttgcaacctcttcaagagc

SEQ ID NO: 165 - DNA, 31 bp, synthetic construct
  primer_bind 1..31: note = A81L forward
  source 1..31: mol_type = other DNA
  modified_base 9: mod_base = OTHER; note = uracil
acctgccctccgacgcccaaatcgagactct

SEQ ID NO: 166 - DNA, 27 bp, synthetic construct
  primer_bind 1..27: note = A81L reverse
  source 1..27: mol_type = other DNA
  modified_base 9: mod_base = OTHER; note = uracil
agggcaggtcggagaggtcgacctcgg

SEQ ID NO: 167 - DNA, 26 bp, synthetic construct
  primer_bind 1..26: note = S110V forward
  source 1..26: mol_type = other DNA
  modified_base 8: mod_base = OTHER; note = uracil
cgtggcctccggccgccgcgtcgccg

SEQ ID NO: 168 - DNA, 27 bp, synthetic construct
  primer_bind 1..27: note = S110V reverse
  source 1..27: mol_type = other DNA
  modified_base 9: mod_base = OTHER; note = uracil
aggccacgtaggaggcaatgaggtcgc

SEQ ID NO: 169 - DNA, 28 bp, synthetic construct
  primer_bind 1..28: note = I129F forward
  source 1..28: mol_type = other DNA
  modified_base 9: mod_base = OTHER; note = uracil
attcgacgtcgcccttgagctcggcatc

SEQ ID NO: 170 - DNA, 27 bp, synthetic construct
  primer_bind 1..27: note = I129F reverse
  source 1..27: mol_type = other DNA
  modified_base 9: mod_base = OTHER; note = uracil
acgtcgaatgcatcagtggcgaaaagg

SEQ ID NO: 171 - DNA, 32 bp, synthetic construct
  primer_bind 1..32: note = I188L forward
  source 1..32: mol_type = other DNA
  modified_base 7: mod_base = OTHER; note = uracil
atttgctggacccggttcaggataggaagaac

SEQ ID NO: 172 - DNA, 26 bp, synthetic construct
  primer_bind 1..26: note = I188L reverse
  source 1..26: mol_type = other DNA
  modified_base 7: mod_base = OTHER; note = uracil
agcaaatccttgccgtggaccggaat

SEQ ID NO: 173 - DNA, 27 bp, synthetic construct
  primer_bind 1..27: note = L333F forward
  source 1..27: mol_type = other DNA
  modified_base 7: mod_base = OTHER; note = uracil
aattcctgcccgaggggttcttggagc

SEQ ID NO: 174 - DNA, 25 bp, synthetic construct
  primer_bind 1..25: note = L333F reverse
  source 1..25: mol_type = other DNA
  modified_base 7: mod_base = OTHER; note = uracil
aggaatttcaacgggtcgatctccc

SEQ ID NO: 175 - DNA, 41 bp, synthetic construct
  primer_bind 1..41: note = M351S forward
  source 1..41: mol_type = other DNA
  modified_base 8: mod_base = OTHER; note = uracil
aggggtttggtcttgccaagctgggccccgcagatcgatgt

SEQ ID NO: 176 - DNA, 26 bp, synthetic construct
  primer_bind 1..26: note = M351S reverse
  source 1..26: mol_type = other DNA
  modified_base 8: mod_base = OTHER; note = uracil
aaacccctgcccgcggtctgctccaa

SEQ ID NO: 177 - DNA, 33 bp, synthetic construct
  primer_bind 1..33: note = F381V fw
  source 1..33: mol_type = other DNA
  modified_base 8: mod_base = OTHER; note = uracil
agcgtggtccatggggtaccactaattacatgg

SEQ ID NO: 178 - DNA, 27 bp, synthetic construct
  primer_bind 1..27: note = F381V reverse
  source 1..27: mol_type = other DNA
  modified_base 8: mod_base = OTHER; note = uracil
accacgctctccagtgttgaattccac

SEQ ID NO: 179 - DNA, 30 bp, synthetic construct
  primer_bind 1..30: note = M388A forward
  source 1..30: mol_type = other DNA
  modified_base 7: mod_base = OTHER; note = uracil
attgcatggcccctctatgcagagcaaaag

SEQ ID NO: 180 - DNA, 25 bp, synthetic construct
  primer_bind 1..25: note = T388A reverse
  source 1..25: mol_type = other DNA
  modified_base 7: mod_base = OTHER; note = uracil
atgcaattagtggtaccccatggaa

SEQ ID NO: 181 - DNA, 30 bp, synthetic construct
  primer_bind 1..30: note = S55A forward
  source 1..30: mol_type = other DNA
  modified_base 9: mod_base = OTHER; note = uracil
agcgcgacttcctctcctccctccctgcct

SEQ ID NO: 182 - DNA, 34 bp, synthetic construct
  primer_bind 1..34: note = S55A reverse
  source 1..34: mol_type = other DNA
  modified_base 9: mod_base = OTHER; note = uracil
agtcgcgctgggaggctgagggcgggccgctggt

SEQ ID NO: 183 - DNA, 32 bp, synthetic construct
  primer_bind 1..32: note = A125G forward
  source 1..32: mol_type = other DNA
  modified_base 7: mod_base = OTHER; note = uracil
accttttcggcactgatgcaatcgacgtcgcc

SEQ ID NO: 184 - DNA, 25 bp, synthetic construct
  primer_bind 1..25: note = A125G reverse
  source 1..25: mol_type = other DNA
  modified_base 7: mod_base = OTHER; note = uracil
aaaaggtcgacgacgagggcggcga

SEQ ID NO: 185 - DNA, 25 bp, synthetic construct
  primer_bind 1..25: note = F140Y forward
  source 1..25: mol_type = other DNA
  modified_base 7: mod_base = OTHER; note = uracil
acatcttcttcccctccaccgccat

SEQ ID NO: 186 - DNA, 26 bp, synthetic construct
  primer_bind 1..26: note = F140Y reverse
  source 1..26: mol_type = other DNA
  modified_base 7: mod_base = OTHER; note = uracil
aagatgtaagggcggatgccgagctc

SEQ ID NO: 187 - DNA, 30 bp, synthetic construct
  primer_bind 1..30: note = GV296/297LG forward
  source 1..30: mol_type = other DNA
  modified_base 7: mod_base = OTHER; note = uracil
actgggtctggcccacagccagcagaggtt

SEQ ID NO: 188 - DNA, 25 bp, synthetic construct
  primer_bind 1..25: note = GV296/297LG reverse
  source 1..25: mol_type = other DNA
  modified_base 7: mod_base = OTHER; note = uracil
acccagtgcaagctcgttctgctgc

SEQ ID NO: 189 - DNA, 38 bp, synthetic construct
  primer_bind 1..38: note = H300M forward
  source 1..38: mol_type = other DNA
  modified_base 9: mod_base = OTHER; note = uracil
aggtgtgctggccatgagccagcagaggttcctatggg

SEQ ID NO: 190 - DNA, 27 bp, synthetic construct
  primer_bind 1..27: note = H300M reverse
  source 1..27: mol_type = other DNA
  modified_base 9: mod_base = OTHER; note = uracil
agcacacctgcaagctcgttctgctgc

SEQ ID NO: 191 - DNA, 34 bp, synthetic construct
  primer_bind 1..34: note = E340G forward
  source 1..34: mol_type = other DNA
  modified_base 9: mod_base = OTHER; note = uracil
aggggttcttgggccagaccgcgggcaggggttt

SEQ ID NO: 192 - DNA, 27 bp, synthetic construct
  primer_bind 1..27: note = E340G reverse
  source 1..27: mol_type = other DNA
  modified_base 9: mod_base = OTHER; note = uracil
agaacccctcgggcaggagtttcaacg

SEQ ID NO: 193 - DNA, 33 bp, synthetic construct
  primer_bind 1..33: note = F381V-Mut88 forward
  source 1..33: mol_type = other DNA
  modified_base 8: mod_base = OTHER; note = uracil
agcgtggtccatggggtaccactaattgcatgg

SEQ ID NO: 194 - DNA, 48 bp, synthetic construct
  primer_bind 1..48: note = A388C/A399C-Mut90 reverse
  source 1..48: mol_type = other DNA
  modified_base 10: mod_base = OTHER; note = Uracil
atcttttgctctgcatagaggggccagcaaattagtggtaccccatgg

SEQ ID NO: 195 - protein, 478 aa, Persicaria tinctoria
MSESPAAPPTTAPPPHVIIVPSAGMGHLIPLAEFAKRLLPRFTFTFAVPTSGPPSSSQRDFLSSLPASIDTSFLPEVDLSDAPSDAQIETLMSLMVVRSLPSLRDLIASYSASGRRVAALVVDLFATDAIDVALELGIRPFIFFPSTAMTLSFFLHLEKLDETVSCEFAELSDPVQIPGCIPVHGKDLIDPVQDRKNDAYKWLLHHSKRYKLAEGVIVNSFEGLEGGPIRELLHPEPGKPRVYPVGPLIQAGSCEKGAAARPECLKWLDQQPRGSVLFVNFGSGGVLSTEQQNELAGVLAHSQQRFLWVVRPPNDGIANATYFSVDGEIDPLKLLPEGFLEQTAGRGLVLPMWAPQIDVLSHESTGGFLTHCGWNSTLESVFHGVPLITWPLYAEQKMNAVMLTEGLRVGLRPSVGKDGIIRGAEIARVIGELMEGEEGKRIRSKMQELKRAASAVLSKDGSSTRALEEVAKIWESKV

SEQ ID NO: 196 - protein, 477 aa, synthetic construct
SESPAAPPTTAPPPHVIIVPSAGMGHLIPLAEFAKRLLPRFTFTFAVPTSGPPSSSQRDFLSSLPASIDTSFLPPVDLSDAPSDAKIETLMSLMVVRSLPSLRDLIASYVASGRRVAALVVDLFATDAIDVALELGIRPFIFFPSTAMTLSFFLHLEKLDETVSCEFAELSDPVQIPGCIPVHGKDLLDPVQDRKNDAYKWLLHHSKRYKLAEGVIVNSFEDLEGGPIRELLHPEPGKPRVYPVGPLIQAGSCEKGAAARPECLKWLDQQPRGSVLFVNFGSGGVLSTEQQNELALGLAHSQQRFLWVVRPPNDGIANATYFSVDGEIDPLKLLPEGFLEQTAGRGLVLPMWAPQIDVLSHESTGGFLTHCGWNSTLESVFHGVPLIAWPLYAEQKMNAVMLTEGLRVGLRPKVGKDGIIRGAEIARVIKELMEGEEGKRIRSKMQELKRAASAVLSKDGSSTRALEEVAKIWESKV

SEQ ID NO: 197 - protein, 477 aa, synthetic construct
SESPAAPPTTAPPPHVIIVPSAGMGHLIPLAEFAKRLLPRFTFTFAVPTSGPPSSSQRDFLSSLPASIDTSFLPPVDLSDAPSDAKIETLMSLMVVRSLPSLRDLIASYVASGRRVAALVVDLFATDAIDVALELGIRPFIFFPSTAMTLSFFLHLEKLDETVSCEFAELSDPVQIPGCIPVHGKDLLDPVQDRKNDAYKWLLHHSKRYKLAEGVIVNSFEDLEGGPIRELLHPEPGKPRVYPVGPLIQAGSCEKGAAARPECLKWLDQQPRGSVLFVNFGSGGVLSTEQQNELALGLAHSQQRFLWVVRPPNDGIANATYFSVDGEIDPLKLLPEGFLEQTAGRGLVLPMWAPQIDVLSHESTGGFLTHCGWNSTLESVVHGVPLIAWPLYAEQKMNAVMLTEGLRVGLRPKVGKDGIIRGAEIARVIKELMEGEEGKRIRSKMQELKRAASAVLSKDGSSTRALEEVAKIWESKV

SEQ ID NO: 198 - protein, 477 aa, synthetic construct
SESPAAPPTTAPPPHVIIVPSAGMGHLIPLAEFAKRLLPRFTFTFAVPTSGPPSSSQRDFLSSLPASIDTSFLPPVDLSDAPSDAKIETLMSLMVVRSLPSLRDLIASYVASGRRVAALVVDLFATDAIDVALELGIRPFIFFPSTAMTLSFFLHLEKLDETVSCEFAELSDPVQIPGCIPVHGKDLLDPVQDRKNDAYKWLLHHSKRYKLAEGVIVNSFEDLEGGPIRELLHPEPGKPRVYPVGPLIQAGSCEKGAAARPECLKWLDQQPRGSVLFVNFGSGGVLSTEQQNELALGLAHSQQRFLWVVRPPNDGIANATYFSVDGEIDPLKLLPEGFLEQTAGRGLVLPMWAPQIDVLSHESTGGFLTHCGWNSTLESVVHGVPLICWPLYAEQKMNCVMLTEGLRVGLRPKVGKDGIIRGAEIARVIKELMEGEEGKRIRSKMQELKRAASAVLSKDGSSTRALEEVAKIWESKV

SEQ ID NO: 199 - DNA, 1437 bp, Persicaria tinctoria
  source 1..1437: mol_type = genomic DNA
  CDS 1..1437: protein_id = 200; translation = MSESPAAPPTTAPPPHVIIMPSAGMGHLIPLAEFAKRLLPRFTF TFAVPTSGPPSSSQRDFLSSLPASIDTSFLPEVDLSDAPSDAQIETLMSLMVVRSLPS LRDLIASYSASGRRVAALVVDLFATDAIDVALELGIRPFIFFPSTAMTLSFFLHLEKL DETVSCEFAELSDPVQIPGCIPVHGKDLIDPVQDRKNDAYKWLLHHSKRYKLAEGVIV NSFEGLEAGPIRQLLHPEPGKPRVYPVGPLIQAGSCEKGAAARPECLKWLDQQPRGSV LFVNFGSGGVLSTEQQNELAGVLAHSQQRFLWVVRPPNDGIANATYFSVDGEIDPLKL LPEGFLEQTAGRGLVLPMWAPQIDVLSHESTGGFLTHCGWNSTLESVFHGVPLITWPL YAEQKMNAVMLTEGLRVGLRPSVGKDGIIRGDEIARVIGELMEGEEGKRIRSKMQELK RAASAVLSKDGSSTRALEEVAKIWESKV
atgagcgaaagcccggcagccccgcctaccaccgcacctcctcctcatgtgattattatgccgagtgcaggtatgggtcatctgattccgctggcagaatttgcaaaacgtctgctgccgcgttttacctttacctttgccgttccgaccagtggtccgccgagtagcagtcagcgtgattttctgagcagtctgccggccagcattgataccagctttctgccggaagttgatctgagtgatgcaccgagcgatgcccagattgaaaccctgatgagcctgatggtggtgcgcagcctgccgagtctgcgtgatctgattgccagttatagtgcaagtggtcgtcgtgtggccgcactggttgtggatctgtttgccaccgatgccattgatgtggccctggaactgggtattcgtccgtttattttctttccgagtaccgcaatgaccctgagctttttcctgcatctggaaaaactggatgaaaccgtgagttgcgaatttgcagaactgagcgatccggtgcagattccgggttgcattccggttcatggtaaagatctgattgatccggtgcaagatcgcaaaaatgatgcctataaatggctgctgcatcatagcaaacgttataaactggcagaaggtgttattgtgaatagttttgaaggcctggaagcaggcccgattcgtcagctgctgcatccggaaccgggtaaaccgcgcgtttatccggtgggcccgctgattcaggccggctcatgcgaaaaaggtgcagccgcacgtccggaatgtctgaaatggctggatcagcagccgcgtggtagtgtgctgtttgtgaattttggtagcggcggcgtgctgagcaccgaacagcagaatgaactggcaggtgttctggcccatagccagcagcgttttctgtgggtggttcgcccgccgaatgatggtattgcaaatgcaacctattttagcgtggatggtgaaattgatccgctgaaactgctgccggaaggttttctggaacagaccgcaggtcgcggtctggttctgccgatgtgggcaccgcagattgatgtgctgagtcatgaaagtaccggcggctttctgacccattgtggttggaatagcaccctggaaagtgtgtttcatggtgttccgctgattacctggccgctgtatgccgaacagaaaatgaatgccgtgatgctgaccgaaggtctgcgcgtgggtctgcgtccgagcgttggtaaagatggtattattcgtggtgacgaaattgcccgtgttattggtgaactgatggaaggtgaagaaggcaaacgcattcgtagcaaaatgcaggaactgaaacgcgcagcaagtgccgttctgagcaaagatggtagtagtacccgtgcactggaagaagtggccaaaatttgggaaagcaaagtgtaa

SEQ ID NO: 200 - protein, 478 aa, Persicaria tinctoria
MSESPAAPPTTAPPPHVIIMPSAGMGHLIPLAEFAKRLLPRFTFTFAVPTSGPPSSSQRDFLSSLPASIDTSFLPEVDLSDAPSDAQIETLMSLMVVRSLPSLRDLIASYSASGRRVAALVVDLFATDAIDVALELGIRPFIFFPSTAMTLSFFLHLEKLDETVSCEFAELSDPVQIPGCIPVHGKDLIDPVQDRKNDAYKWLLHHSKRYKLAEGVIVNSFEGLEAGPIRQLLHPEPGKPRVYPVGPLIQAGSCEKGAAARPECLKWLDQQPRGSVLFVNFGSGGVLSTEQQNELAGVLAHSQQRFLWVVRPPNDGIANATYFSVDGEIDPLKLLPEGFLEQTAGRGLVLPMWAPQIDVLSHESTGGFLTHCGWNSTLESVFHGVPLITWPLYAEQKMNAVMLTEGLRVGLRPSVGKDGIIRGDEIARVIGELMEGEEGKRIRSKMQELKRAASAVLSKDGSSTRALEEVAKIWESKV

SEQ ID NO: 201 - DNA, 1437 bp, Persicaria tinctoria
  source 1..1437: mol_type = genomic DNA
  CDS 1..1437: protein_id = 202; translation = MSESPAAPPTTAPPPHVIIMPSAGMGHLIPLAEFAKRLLPRFTF TFAVPTSGPPSSSQRDFLSSLPASIDTSFLPEVDLSDAPSDAQIETLMSLMVVRSLPS LRDLIASYSASGRRVAALVVDLFATDAIDVALELGIRPFIFFPSTAMTLSFFLHLEKL DETVSCEFAELSDPVQIPGCIPVHGKDLIDPVQDRKNDAYKWLLHHSKRYKLAEGVIV NSFEGLEGGPIRELLHPEPGKPRVYPVGPLIQAGSCEKGAAARPECLKWLDQQPRGSV LFVNFGSGGVLSTEQQNELAGVLAHSQQRFLWVVRPPNDGIANATYFSVDGEIDPLKL LPEGFLEQTAGRGLVLPMWAPQIDVLSHESTGGFLTHCGWNSTLESVFHGVPLITWPL YAEQKMNAVMLTEGLRVGLRPSVGKDGIIRGDEIARVIGELMEGEEGKRIRSKMQELK RAASAVLSKDGSSTRALEEVAKIWESKV
atgagcgaaagcccggccgccccgcctaccacagctcctcctcctcatgtgattattatgccgagtgcaggtatgggtcatctgattccgctggcagaatttgccaaacgcctgctgccgcgttttacctttacctttgcagtgccgaccagtggtccgccgagtagcagtcagcgcgattttctgagcagtctgccggcaagcattgataccagctttctgccggaagttgatctgagtgatgcaccgagtgatgcacagattgaaaccctgatgagcctgatggttgttcgtagcctgccgagtctgcgcgatctgattgcaagctatagtgccagcggtcgtcgcgttgcagcactggttgtggatctgtttgcaaccgatgcaattgatgtggcactggaactgggcattcgtccgtttattttctttccgagcaccgccatgaccctgagctttttcctgcatctggaaaaactggatgaaaccgttagctgcgaatttgcagaactgagtgatccggttcagattccgggttgtattccggtgcatggtaaagatctgattgatccggttcaagatcgtaaaaatgatgcctataaatggctgctgcatcatagcaaacgctataaactggcagaaggcgtgattgttaatagctttgaaggcctggaaggcggtccgattcgtgaactgctgcatccggaaccgggcaaaccgcgtgtgtatccggttggtccgctgattcaggcaggtagttgtgaaaaaggtgccgcagcacgcccggaatgtctgaaatggctggatcagcagccgcgtggtagtgttctgtttgttaattttggcagcggcggtgttctgagcaccgaacagcagaatgaactggcaggtgtgctggcacatagccagcagcgttttctgtgggttgtgcgtccgccgaatgatggtattgccaatgcaacctattttagcgttgatggcgaaattgatccgctgaaactgctgccggaaggctttctggaacagaccgcaggtcgtggtctggttctgccgatgtgggcaccgcagattgatgttctgagccatgaaagcaccggcggctttctgacccattgcggctggaatagcaccctggaaagtgtttttcatggtgtgccgctgattacctggccgctgtatgccgaacagaaaatgaatgccgttatgctgaccgaaggtctgcgtgtgggcctgcgtccgagtgttggtaaagatggtattattcgcggtgacgaaattgcccgtgttattggcgaactgatggaaggtgaagaaggcaaacgtattcgtagcaaaatgcaggaactgaaacgtgccgccagcgccgttctgagtaaagatggcagcagcacccgtgccctggaagaagttgcaaaaatttgggaaagcaaagtgtaa

SEQ ID NO: 202 - protein, 478 aa, Persicaria tinctoria
MSESPAAPPTTAPPPHVIIMPSAGMGHLIPLAEFAKRLLPRFTFTFAVPTSGPPSSSQRDFLSSLPASIDTSFLPEVDLSDAPSDAQIETLMSLMVVRSLPSLRDLIASYSASGRRVAALVVDLFATDAIDVALELGIRPFIFFPSTAMTLSFFLHLEKLDETVSCEFAELSDPVQIPGCIPVHGKDLIDPVQDRKNDAYKWLLHHSKRYKLAEGVIVNSFEGLEGGPIRELLHPEPGKPRVYPVGPLIQAGSCEKGAAARPECLKWLDQQPRGSVLFVNFGSGGVLSTEQQNELAGVLAHSQQRFLWVVRPPNDGIANATYFSVDGEIDPLKLLPEGFLEQTAGRGLVLPMWAPQIDVLSHESTGGFLTHCGWNSTLESVFHGVPLITWPLYAEQKMNAVMLTEGLRVGLRPSVGKDGIIRGDEIARVIGELMEGEEGKRIRSKMQELKRAASAVLSKDGSSTRALEEVAKIWESKV